(19) United States
(12) Patent Application Publication
(10) Pub. No.: US 2022/0339141 A1
(43) Pub. Date: Oct. 27, 2022

(54) COMBINATION TREATMENT WITH A P53 REACTIVATOR AND AN INHIBITOR OF AN ANTIAPOPTOTIC BCL-2 FAMILY PROTEIN

(71) Applicant: Aprea Therapeutics AB, Solna (SE)

(72) Inventors: Lars Abrahmsén, Solna (SE); Anders Wennborg, Solna (SE)

(21) Appl. No.: 17/760,644

(22) PCT Filed: Sep. 18, 2020
(86) PCT No.: PCT/EP2020/076126

§ 371 Date: Mar. 15, 2022

Related U.S. Application Data
(60) Provisional application No. 63/028,477, filed on May 21, 2020.
(60) Provisional application No. 62/993,509, filed on Mar. 23, 2020.
(60) Provisional application No. 62/902,214, filed on Sep. 18, 2019.

(51) Int. Cl. A61K 31/439; A61K 31/519; A61P 35/02; C07K 16/28

(52) U.S. Cl. CPC A61K 31/439; A61K 31/519; A61P 35/02; C07K 16/2887

(57) ABSTRACT

Provided herein are methods of treating hyperproliferative malignancy in a subject using combination of a compound that can result in reactivation of mutant p53 and a Bcl-2 inhibitor or a Mcl-1 inhibitor. Also provided herein are methods of treating lymphoma in a subject using a combination therapy of a p53 reactivator, a Bcl 2 inhibitor, and rituximab.

### 1. CROSS REFERENCE

This application claims the benefit of U.S. Provisional Patent Application No. 62/902,214, filed Sep. 18, 2019, U.S. Provisional Patent Application No. 62/993,509, filed Mar. 23, 2020, and U.S. Provisional Patent Application No. 63/028,477, filed May 21, 2020, the content of each which is incorporated by reference herein in its entirety.

### 2. FIELD

Provided herein are combination therapies using a p53 reactivator in combination with an inhibitor of an antiapoptotic Bcl-2 family protein (e.g., a Bcl-2 inhibitor or a Mcl-1 inhibitor) for treating a disease or disorder. Also provided herein are combination therapies using a p53 reactivator in combination with a Bcl-2 inhibitor and rituximab (RITUXAN®) for treating a disease or disorder such as lymphoma.

### 3. BACKGROUND

P53 plays a critical role as a tumor suppressor and its gene TP53 is a common target for mutations in tumors. p53 halts the cell cycle and/or triggers apoptosis in response to various stress stimuli, including DNA damage, hypoxia, and oncogene activation (Ko, L. J. & Prives, C., Genes Dev. 10, 1054-1072 (1996); Sherr, C. J., Genes Dev. 12, 2984-2991 (1998)).

Both p53-induced cell cycle arrest and apoptosis could be involved in p53-mediated tumor suppression. A significant proportion of human tumors make a “mutant” p53 protein due to a TP53 mutation, making it highly desirable to restore the wild type p53 activity to yield growth suppression to tumors. Tumor cells are particularly sensitive to p53 reactivation, supposedly for two main reasons. First, tumor cells are sensitized to apoptosis due to oncogene activation (reviewed in Evan, G. & Littlewood, T., Science. 281, 1317-1322 (1998)). Second, mutant p53 proteins tend to accumulate at high levels in tumor cells. Therefore, restoration of the wild type function to the abundant and presumably “activated” mutant p53 should trigger a massive apoptotic response in already sensitized tumor cells, whereas normal cells that harbor low or undetectable levels of p53 should not be affected.

In normal cells p53 is found at very low levels due to negative feedback regulation of the level, unless undergoing developmental or maturation processes involving p53.

Non-Hodgkin lymphomas (NHL) are lymphoid malignant neoplasms with diverse biological and clinical behavior, variously derived from the clonal expansion of B cells, T cells, natural killer cells or precursors of these cells. Chronic lymphocytic leukemia (CLL) is one of the most common types of B-cell NHL, characterized by a progressive accumulation of functionally incompetent monoclonal lymphocytes (Siegel, R. L., et al., CA Cancer J Clin. 70(1), 7-30 (2020)). The incidence rates among men and women in the United States are approximately 6.75 and 3.65 cases per 100,000 population per year, respectively (Yamamoto, J. F., et al., Cancer Causes Control. 19(4), 379-390 (2008); Fitzmaurice, C., et al., JAMA Oncol. 3(4), 524-548, (2017)).

Mantle cell lymphoma (MCL) is another type of mature B-cell NHL, which comprises about 7% of adult NHL in the United States with an incidence of approximately 4 to 8 cases per million persons per year (Harris, N. L., et al., Blood 84(5), 1361-1392 (1994); Armitage, J. O., et al., J Clin Oncol. 16(8), 2780-2795 (1998); Zhou, Y., et al., Cancer 113(4), 791-798 (2008)).

Therapeutically useful compounds have previously been generated based on showing mutant p53 dependent anti-proliferative activity in a cellular assay, including the compound PRIMA-1 (i.e. 2,2-bis(hydroxymethyl)quinuclidin-3-one) (disclosed in WO 02/24692), and its analogs (such as those disclosed in WO 03/070250). Nonetheless, there still remains a general need of effective combination therapies using these compounds in combination with a second therapeutic agent for treating cancer and other diseases or disorders.

### 4. SUMMARY

In one aspect, provided herein is a method of treating hyperproliferative malignancy in a subject, comprising administering to the subject a therapeutically effective amount of a compound that can give reactivation of a mutant p53 and an inhibitor of an antiapoptotic Bcl-2 family protein.

In some embodiments, the compound that can give reactivation of the mutant p53 promotes proper folding of the mutant p53 and restores at least part of a normal p53 function.

In some embodiments, the compound is resting in a shift of the equilibrium from unfolded towards a wild-type like p53 conformation.

In some embodiments, the compound that give reactivation of the mutant p53 interferes with aggregation of misfolded mutant p53 or reduce aggregation of the mutant p53.

In some embodiments, the compound or metabolite or degradation product thereof restores a p53 wild type function by covalent binding to the mutant p53.

In some embodiments, the compound binds to thiol groups in the core domain of the mutant p53 and restore wild-type conformation.

In some embodiments, the mutant p53 comprises at least one of the replacements R175H or R273H.

In some embodiments, the compound that reactivates the mutant p53 is selected from the group consisting of:
2-(hydroxymethyl)-2-(methoxymethyl)quinuclidin-3-one;
2,2-bis(hydroxymethyl)quinuclidin-3-one;
2,2,2-trichloro-N-ethyl-N-((3-oxoquinuclidin-2-yl)methyl)acetamide;
2,2,2-trichloro-N-((3-oxoquinuclidin-2-yl)methyl)acetamide;
N-ethyl-2,2,2-trifluoro-N-((3-oxoquinuclidin-2-yl)methyl)acetamide;
2,2,2-trifluoro-N-((3-oxoquinuclidin-2-yl)methyl)acetamide;
2,2-difluoro-N-((3-oxoquinuclidin-2-yl)methyl)acetamide,
N-((3-oxoquinuclidin-2-yl)methyl)pyridine-3-sulfonamide;
4-fluoro-N-((3-oxoquinuclidin-2-yl)methyl)benzenesulfonamide;
N-ethyl-N-((3-oxoquinuclidin-2-yl)methyl)methanesulfonamide;
N-((3-oxoquinuclidin-2-yl)methyl)methanesulfonamide;
N-((3-oxoquinuclidin-2-yl)methyl)benzenesulfonamide;
2-(N-((3-oxoquinuclidin-2-yl)methyl)methylsulfonamido)acetamide;
N-(methylsulfonyl)-N-((3-oxoquinuclidin-2-yl)methyl)glycine;
N-((3-oxoquinuclidin-2-yl)methyl)pyridine-4-sulfonamide;
N-((3-oxoquinuclidin-2-yl)methyl)pyridine-2-sulfonamide;
N-ethyl-1,1,1-trifluoro-N-((3-oxoquinuclidin-2-yl)-methyl)methanesulfonamide;
1,1,1-trifluoro-N-((3-oxoquinuclidin-2-yl)methyl)methanesulfonamide;
N,N-bis((3-oxoquinuclidin-2-yl)methyl)methanesulfonamide;
N-((3-oxoquinuclidin-2-yl)methyl)propane-2-sulfonamide;
N-((3-oxoquinuclidin-2-yl)methyl)cyclopropanesulfonamide;
1-methyl-N-((3-oxoquinuclidin-2-yl)methyl)cyclopropane-1-sulfonamide;
N-cyclopropyl-N-((3-oxoquinuclidin-2-yl)methyl)methanesulfonamide;
N-((3-oxoquinuclidin-2-yl)methyl)-N-phenylmethanesulfonamide;
1-((3-oxoquinuclidin-2-yl)methyl)pyrimidine-2,4(1H,3H)-dione;
5-methyl-1-((3-oxoquinuclidin-2-yl)methyl)pyrimidine-2,4(1H,3H)-dione;
tert-butyl 5-methyl-2,6-dioxo-3-((3-oxoquinuclidin-2-yl)methyl)-3,6-dihydropyrimidine-1(2H)-carboxylate;
5-methyl-1,3-bis((3-oxoquinuclidin-2-yl)methyl)pyrimidine-2,4(1H,3H)-dione;
N-methyl-1-((3-oxoquinuclidin-2-yl)methyl)-1H-1,2,4-triazole-3-carboxamide;
2-((3-chloro-1H-1,2,4-triazol-1-yl)methyl)quinuclidin-3-one;
N,N-dimethyl-1-((3-oxoquinuclidin-2-yl)methyl)-1H-1,2,4-triazole-3-carboxamide;
2-((1H-1,2,4-triazol-1-yl)methyl)quinuclidin-3-one;
1-((3-oxoquinuclidin-2-yl)methyl)-1H-1,2,4-triazole-3-carbonitrile; and
1-((3-oxoquinuclidin-2-yl)methyl)-1H-1,2,4-triazole-3-carboxamide, or a pharmaceutically acceptable salt thereof.

In some embodiments, the compound is 2-(hydroxymethyl)-2-(methoxymethyl) quinuclidin-3-one (APR-246) having the following formula:

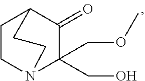

or a pharmaceutically acceptable salt thereof.

In some embodiments, the compound is 2,2,2-trifluoro-N-((3-oxoquinuclidin-2-yl)methyl)acetamide (Compound A) having the following formula:

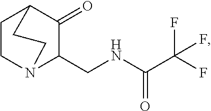 [SMILES: O=C1C2CCN(CC2)C1CNC(=O)C(F)(F)F]

or a pharmaceutically acceptable salt thereof.

In some embodiments, the compound is 2,2,2-trichloro-N-ethyl-N-((3-oxoquinuclidin-2-yl)methyl)acetamide (Compound B) having the following formula:

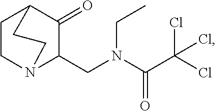 [SMILES: CCN(CC1C(=O)C2CCN1CC2)C(=O)C(Cl)(Cl)Cl]

or a pharmaceutically acceptable salt thereof.

In some embodiments, the compound is 2,2,2-trichloro-N-((3-oxoquinuclidin-2-yl)methyl)acetamide (Compound C) having the following formula:

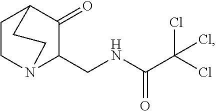 [SMILES: O=C1C2CCN(CC2)C1CNC(=O)C(Cl)(Cl)Cl]

or a pharmaceutically acceptable salt thereof.

In some embodiments, the compound is N-ethyl-2,2,2-trifluoro-N-((3-oxoquinuclidin-2-yl)methyl)acetamide (Compound D) having the following formula:

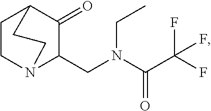 [SMILES: CCN(CC1C(=O)C2CCN1CC2)C(=O)C(F)(F)F]

or a pharmaceutically acceptable salt thereof.

In some embodiments, the compound is 2,2-difluoro-N-((3-oxoquinuclidin-2-yl)methyl)acetamide (Compound E) having the following formula:

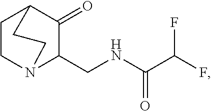 [SMILES: O=C(NCC1C(=O)C2CCN1CC2)C(F)F]

or a pharmaceutically acceptable salt thereof.

In some embodiments, the inhibitor of an antiapoptotic Bcl-2 family protein is a Bcl-2 inhibitor. In some embodiments, the Bcl-2 inhibitor is selected from the group consisting of venetoclax (ABT-199), navitoclax, oblimersen, PNT2258, and SPC2996. In a specific embodiment, the Bcl-2 inhibitor is venetoclax (ABT-199).

In a specific embodiment, the method provided herein comprises administering to the subject a therapeutically effectively amount of APR-246 and venetoclax (ABT-199).

In another specific embodiment, the method provided herein comprises administering to the subject a therapeutically effectively amount of Compound A and venetoclax (ABT-199).

In another specific embodiment, the method provided herein comprises administering to the subject a therapeutically effectively amount of Compound B and venetoclax (ABT-199).

In another specific embodiment, the method provided herein comprises administering to the subject a therapeutically effectively amount of Compound C and venetoclax (ABT-199).

In another specific embodiment, the method provided herein comprises administering to the subject a therapeutically effectively amount of Compound D and venetoclax (ABT-199).

In another specific embodiment, the method provided herein comprises administering to the subject a therapeutically effectively amount of Compound E and venetoclax (ABT-199).

In other embodiments, the inhibitor of an antiapoptotic Bcl-2 family protein is a Mcl-1 inhibitor. In some embodiments, the Mcl-1 inhibitor is selected from the group consisting of AT101, TW-37, Gambogic acid, Sabutoclax (BI-97C1), Marinopyrrole A (maritoclax), UMI-77, A-1210477, MIK665, AMG-176, AZD5991, Flavopiridol, Roscovitine, CR8, Voruciclib (P1446A-05), Cardiac glycosides UNB S1450, Benzyl isothiocyanate, BAY43-9006, BEZ235 AZD8055, and Arsenic trioxide Bufalin. In a specific embodiment, the Mcl-1 inhibitor is AMG-176. In another specific embodiment, the Mcl-1 inhibitor is MIK665.

In a specific embodiment, the method provided herein comprises administering to the subject a therapeutically effectively amount of APR-246 and AMG-176.

In another specific embodiment, the method provided herein comprises administering to the subject a therapeutically effectively amount of APR-246 and MIK665.

In some embodiments, the p53 reactivator is formulated in a first pharmaceutical composition and the inhibitor of an antiapoptotic Bcl-2 family protein is formulated in a second pharmaceutical composition.

In some embodiments, the method provided herein further comprises administering to the subject an additional agent. In a specific embodiment, the additional agent is Azacitidine. In a specific embodiment, the method provided herein comprises administering a subject APR-246, ABT-199 and Azacitidine.

In some embodiments, the method provided herein further comprises administering to the subject an additional agent. In some embodiments, the additional agent is a hypomethylating agent. In a specific embodiment, the additional agent is Azacitidine. In some embodiments, the additional agent is an anti-CD20 antibody. In a specific embodiment, the additional agent is rituximab.

In some embodiments, the hyperproliferative malignancy is a hematological malignancy. In some embodiments, the hematological malignancy is leukemia, lymphoma, or myeloma. In some embodiments, the hematological malignancy is selected from the group consisting of: Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the hematologic malignancy is myelodysplastic syndromes (MDS). In another specific embodiment, the hematologic malignancy is acute myeloid leukemia (AML). In another specific embodiment, the hematologic malignancy is chronic lymphocytic leukemia (CLL). In yet another specific embodiment, the hematologic malignancy is multiple myeloma (MM).

In some embodiments, the hyperproliferative malignancy is a solid tumor cancer. In some embodiments, the solid tumor cancer is selected from the group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

In some embodiments, the hyperproliferative malignancy comprises a cancer cell having mutant p53. In other embodiments, the hyperproliferative malignancy does not comprise a cancer cell having mutant p53. In yet other embodiments, the hyperproliferative malignancy comprises a cancer cell having wild type p53.

In another aspect, provided herein is a method of treating hyperproliferative malignancy in a subject, comprising administering to the subject a therapeutically effective amount of a compound that can give reactivation of a mutant p53, wherein the hyperproliferative malignancy does not comprise a cancer cell having mutant p53 or the hyperproliferative malignancy comprises a cancer cell having wild type p53. In a specific embodiment, the compound is APR-246. In another specific embodiment, the compound is Compound A.

In yet another specific embodiment, the compound is Compound B.

In yet another specific embodiment, the compound is Compound C.

In yet another specific embodiment, the compound is Compound D.

In yet another specific embodiment, the compound is Compound E.

In another aspect, provided herein are methods of treating lymphoma in a subject, comprising administering to the subject: (i) a p53 reactivator; (ii) a Bcl-2 inhibitor; and (iii) an anti-CD20 antibody.

In another aspect, provided herein are methods of treating lymphoma in a subject, comprising administering to the subject: (i) a p53 reactivator; (ii) a Bcl-2 inhibitor; and (iii) rituximab.

In certain embodiments, the p53 reactivator is a compound that can give reactivation of a mutant p53, or a degradation product thereof that can give reactivation of a mutant p53.

In certain embodiments, the mutant p53 comprises a mutation selected from the group consisting of R248Q, R248W, R273H, R273C, R175H, Y220C, G245S, R249S, and R282W.

In certain embodiments, the p53 reactivator is capable of reactivating the mutant p53 to restore at least 10%, at least 20%, at least 30%, at least 40%, at least 50%, at least 60%, at least 70%, at least 80%, or at least 90% of wild type p53 activity.

In certain embodiments, the compound or degradation product thereof promotes proper folding of mutant and wild-type p53 proteins.

In certain embodiments, the compound or degradation product thereof is capable of shifting the equilibrium from unfolded towards a folded structure of wild-type or mutant p53, or wherein the compound or degradation product thereof is capable of interfering with aggregation of misfolded wild-type or mutant p53, or wherein the compound or degradation product thereof is capable of reducing aggregation of the wild-type or mutant p53.

In certain embodiments, the compound or degradation product thereof is capable of promoting a folded structure of wild-type or mutant p53, and/or is capable of restoring or enhancing wild type function by covalent binding to the wild-type or mutant p53.

In certain embodiments, the compound or degradation product threreof is capable of binding to thiol groups in the core domain of wild-type or mutant p53 and promote a folded conformation.

In certain embodiments, the compound that can give reactivation of mutant p53 is selected from the group consisting of:
2-(hydroxymethyl)-2-(methoxymethyl)quinuclidin-3-one;
2,2-bis(hydroxymethyl)quinuclidin-3-one;
2,2,2-trichloro-N-ethyl-N-((3-oxoquinuclidin-2-yl)methyl)acetamide;
2,2,2-trichloro-N-((3-oxoquinuclidin-2-yl)methyl)acetamide;
N-ethyl-2,2,2-trifluoro-N-((3-oxoquinuclidin-2-yl)methyl)acetamide;
2,2,2-trifluoro-N-((3-oxoquinuclidin-2-yl)methyl)acetamide;
2,2-difluoro-N-((3-oxoquinuclidin-2-yl)methyl)acetamide,
N-((3-oxoquinuclidin-2-yl)methyl)pyridine-3-sulfonamide;
4-fluoro-N-((3-oxoquinuclidin-2-yl)methyl)benzenesulfonamide;
N-ethyl-N-((3-oxoquinuclidin-2-yl)methyl)methanesulfonamide;
N-((3-oxoquinuclidin-2-yl)methyl)methanesulfonamide;
N-((3-oxoquinuclidin-2-yl)methyl)benzenesulfonamide;
2-(N-((3-oxoquinuclidin-2-yl)methyl)methylsulfonamido)acetamide;
N-(methylsulfonyl)-N-((3-oxoquinuclidin-2-yl)methyl)glycine;
N-((3-oxoquinuclidin-2-yl)methyl)pyridine-4-sulfonamide;
N-((3-oxoquinuclidin-2-yl)methyl)pyridine-2-sulfonamide;
N-ethyl-1,1,1-trifluoro-N-((3-oxoquinuclidin-2-yl)-methyl)methanesulfonamide;
1,1,1-trifluoro-N-((3-oxoquinuclidin-2-yl)methyl)methanesulfonamide;
N,N-bis((3-oxoquinuclidin-2-yl)methyl)methanesulfonamide;
N-((3-oxoquinuclidin-2-yl)methyl)propane-2-sulfonamide;
N-((3-oxoquinuclidin-2-yl)methyl)cyclopropanesulfonamide;
1-methyl-N-((3-oxoquinuclidin-2-yl)methyl)cyclopropane-1-sulfonamide;
N-cyclopropyl-N-((3-oxoquinuclidin-2-yl)methyl)methanesulfonamide;
N-((3-oxoquinuclidin-2-yl)methyl)-N-phenylmethanesulfonamide;
1-((3-oxoquinuclidin-2-yl)methyl)pyrimidine-2,4(1H,3H)-dione;
5-methyl-1-((3-oxoquinuclidin-2-yl)methyl)pyrimidine-2,4(1H,3H)-dione;
tert-butyl 5-methyl-2,6-dioxo-3-((3-oxoquinuclidin-2-yl)methyl)-3,6-dihydropyrimidine-1(2H)-carboxylate;
5-methyl-1,3-bis((3-oxoquinuclidin-2-yl)methyl)pyrimidine-2,4(1H,3H)-dione;
N-methyl-1-((3-oxoquinuclidin-2-yl)methyl)-1H-1,2,4-triazole-3-carboxamide;
2-((3-chloro-1H-1,2,4-triazol-1-yl)methyl)quinuclidin-3-one;
N,N-dimethyl-1-((3-oxoquinuclidin-2-yl)methyl)-1H-1,2,4-triazole-3-carboxamide;
2-((1H-1,2,4-triazol-1-yl)methyl)quinuclidin-3-one;
1-((3-oxoquinuclidin-2-yl)methyl)-1H-1,2,4-triazole-3-carbonitrile; and
1-((3-oxoquinuclidin-2-yl)methyl)-1H-1,2,4-triazole-3-carboxamide;
   or a pharmaceutically acceptable salt thereof.

In certain embodiments, the compound is 2-(hydroxymethyl)-2-(methoxymethyl)quinuclidin-3-one (APR-246) having the following formula:

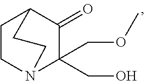

or a pharmaceutically acceptable salt thereof.

In certain embodiments, the compound is a compound selected from the group consisting of:
2,2,2-trifluoro-N-((3-oxoquinuclidin-2-yl)methyl)acetamide (Compound A);
2,2,2-trichloro-N-ethyl-N-((3-oxoquinuclidin-2-yl)methyl)acetamide (Compound B);
2,2,2-trichloro-N-((3-oxoquinuclidin-2-yl)methyl)acetamide (Compound C);
N-ethyl-2,2,2-trifluoro-N-((3-oxoquinuclidin-2-yl)methyl)acetamide (Compound D); and
2,2-difluoro-N-((3-oxoquinuclidin-2-yl)methyl)acetamide (Compound E), or a pharmaceutically acceptable salt thereof. The structures of Compounds A-E are shown in Table 1 below in Section 6.2.1.

In certain embodiments, the compound is 2,2,2-trifluoro-N-((3-oxoquinuclidin-2-yl)methyl)acetamide (Compound A), or a pharmaceutically acceptable salt thereof.

In certain embodiments, the compound is 2,2,2-trichloro-N-ethyl-N-((3-oxoquinuclidin-2-yl)methyl)acetamide (Compound B), or a pharmaceutically acceptable salt thereof.

In certain embodiments, the compound is 2,2,2-trichloro-N-((3-oxoquinuclidin-2-yl)methyl)acetamide (Compound C), or a pharmaceutically acceptable salt thereof.

In certain embodiments, the compound is N-ethyl-2,2,2-trifluoro-N-((3-oxoquinuclidin-2-yl)methyl)acetamide (Compound D), or a pharmaceutically acceptable salt thereof.

In certain embodiments, the compound is 2,2-difluoro-N-((3-oxoquinuclidin-2-yl)methyl)acetamide (Compound E), or a pharmaceutically acceptable salt thereof.

In certain embodiments, the Bcl-2 inhibitor is selected from the group consisting of venetoclax (ABT-199), navitoclax, oblimersen, PNT2258, and SPC2996.

In certain embodiments, the Bcl-2 inhibitor is venetoclax (ABT-199).

In certain embodiments, the anti-CD20 antibody is selected from the group consisting of rituximab, obinutuzumab, ocaratuzumab, ofatumumab, ocrelizumab, tositumomab, ibritumomab, ibritumomab tiuxetan, ublituximab, and veltuzumab. In a preferred embodiment, the anti-CD20 antibody is rituximab.

In certain embodiments, the method comprises administering to the subject a therapeutically effectively amount of APR-246, venetoclax (ABT-199), and rituximab.

In certain embodiments, APR-246 is administered at a dose of about 4.5 g/day for 4 days, venetoclax (ABT-199) is administered daily at a dose of about 400 mg, and rituximab is administered once at a dose of about 375 mg/m² or 500 mg/m² in each 28-day cycle.

In certain embodiments, APR-246 is administered at a dose of about 4.0 g/day for 4 days, venetoclax (ABT-199) is administered daily at a dose of about 400 mg, and rituximab is administered once at a dose of about 375 mg/m² or 500 mg/m² in each 28-day cycle.

In certain embodiments, APR-246 is administered at a dose of about 3.5 g/day for 4 days, venetoclax (ABT-199) is administered daily at a dose of about 400 mg, and rituximab is administered once at a dose of about 375 mg/m² or 500 mg/m² in each 28-day cycle.

In certain embodiments, APR-246 is administered on Days 1-4, venetoclax (ABT-199) is administered daily, and rituximab is administered on Day 5 of each 28-day cycle.

In certain embodiments, APR-246, venetoclax (ABT-199), and rituximab are administered for 1 to 20 cycles.

In certain embodiments, the p53 reactivator is formulated in a first pharmaceutical composition, the Bcl-2 inhibitor is formulated in a second pharmaceutical composition, and rituximab is formulated in a third pharmaceutical composition.

In certain embodiments, the lymphoma is a Hodgkin lymphoma (HL) or a non-Hodgkin lymphoma (NHL).

In certain embodiments, the lymphoma is a non-Hodgkin lymphoma (NHL).

In certain embodiments, the non-Hodgkin lymphoma (NHL) is a mature (peripheral) B-cell neoplasm.

In certain embodiments, the non-Hodgkin lymphoma (NHL) is selected from the group consisting of: chronic lymphocytic leukemia (CLL), mantle cell lymphoma (MCL), B-cell prolymphocytic leukemia, diffuse large cell B-cell lymphoma (DLBCL), lymphoplasmacytic lymphoma, splenic marginal zone B-cell lymphoma, hairy cell leukemia, plasma cell myeloma (plasmacytoma), extranodal marginal zone B-cell lymphoma, nodal marginal zone lymphoma, follicle center lymphoma, and Burkitt's leukemia (Burkitt's lymphoma).

In certain embodiments, the lymphoma is chronic lymphocytic leukemia (CLL) or mantle cell lymphoma (MCL).

In certain embodiments, the lymphoma is chronic lymphocytic leukemia (CLL).

In certain embodiments, the lymphoma is mantle cell lymphoma (MCL).

In certain embodiments, the non-Hodgkin lymphoma (NHL) is relapsed or refractory NHL.

In certain embodiments, the non-Hodgkin lymphoma (NHL) is relapsed or refractory chronic lymphocytic leukemia (CLL) or mantle cell lymphoma (MCL).

In certain embodiments, the non-Hodgkin lymphoma (NHL) is relapsed or refractory CLL.

In certain embodiments, the non-Hodgkin lymphoma (NHL) is relapsed or refractory MCL.

In certain embodiments, the lymphoma comprises a cancer cell having mutant p53.

In certain embodiments, the method provided herein further comprises determining by gene sequencing if the subject has TP53 mutation.

In certain embodiments, the subject is not treated with any Bcl-2 inhibitor prior to the co-administration of the p53 reactivator, the Bcl-2 inhibitor, and rituximab.

### 5. BRIEF DESCRIPTION OF THE FIGURES

FIG. 1 depicts the study scheme for the Phase I/II study described in Example 2 in Section 7 below.

FIG. 2 depicts dose-finding study design for the Phase I/II study described in Example 2 in Section 7 below.

FIG. 3 depicts the study scheme for the Phase I/II and dose expansion study of APR-246 in combination with venetoclax and rituximab in subjects with TP53-mutant Relapsed and/or Refractory non-Hodgkin lymphoma including chronic lymphocytic leukemia (CLL) and mantle cell lymphoma (MCL). Venetoclax-R refers to venetoclax+rituximab.

### 6. DETAILED DESCRIPTION

The present disclosure is based in part on the surprising finding that the combination of a p53 reactivator and an inhibitor of an antiapoptotic Bcl-2 family protein (e.g., a Bcl-2 inhibitor or a Mcl-1 inhibitor) produces synergistic effects in treating certain diseases and disorders.

Provided herein are combination treatments with an agonist of p53 such as a p53 reactivator (see Section 6.2.1) with a Bcl-2 inhibitor (see Section 6.2.2) or with a Mcl-1 Inhibitor (see Section 6.2.3) see Section). Also provided herein are combination treatments with an agonist of p53 such as a p53 reactivator (see Section 6.2.1) with a Bcl-2 inhibitor (see Section 6.2.2) and an antibody binding CD20 (aka anti-CD20 antibody; e.g., monoclonal antibody (mAb) rituximab (RITUXAN®); see Section 6.2.4) for the treatment of a lymphoma. See Section 6.4. Manners of administration and dosing regimen are described in Section 6.4.

6.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); and Current Protocols in Molecular Biology (Ausubel et al. eds., 2003).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “effective amount” or “therapeutically effective amount” as used herein refers to the amount of a therapeutic compound, a combination of therapeutic compounds or pharmaceutical compositions thereof provided herein, which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or disorder resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease.

The terms “prevent,” “preventing,” and “prevention” refer to reducing the likelihood of the onset (or recurrence) of a disease, disorder, condition, or associated symptom(s).

The term “a mutant p53 mediated disease or disorder” as used herein refers to a disease or disorder that is caused or partically caused by mutation of the p53 gene (TP53). For example, a mutant p53 mediated cancer means the cancer that contains a cell having a mutant TP53.

As used herein, the term “alkyl” unless otherwise stated, means an unbranched or branched, saturated or unsaturated (alkenyl or alkynyl) hydrocarbyl radical. The term “Cx-Cy alkyl” means a straight or branched chain hydrocarbon containing x to y carbon atoms. For example, “C2-C6 alkyl” means a straight or branched chain hydrocarbon containing 2 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “aryl” means an aromatic group, such as phenyl or naphthyl.

As used herein, the term “cycloalkyl” means a monocyclic or bicyclic, saturated or partially unsaturated (but not aromatic), hydrocarbon ring of three to ten carbon ring atoms. Cycloalkyl groups include fused and bridged bicyclic rings. For example, when fused, the cycloalkyl group may comprise two rings that share adjacent atoms (e.g., one covalent bond). When bridged, the cycloalkyl group may comprise two rings that share three or more atoms, separating the two bridgehead atoms by a bridge containing at least one atom. When a cycloalkyl group contains from x-y ring carbon atoms, it may be referred to herein as Cx-Cy cycloalkyl. In certain embodiments, cycloalkyl is C3-C10 cycloalkyl, or is C5-C7 cycloalkyl, or is C5-C6 cycloalkyl, or is C3-C6 cycloalkyl, or is C3-C7 cycloalkyl. In certain embodiments, cycloalkyl is C3-C8 cycloalkyl. In certain embodiments, cycloalkyl groups include cyclopropyl, cyclobutyl, cyclopentyl, or cyclohexyl.

As used herein, the term “heteroaryl” means a mono-, bi-, or tricyclic heteroaromatic group containing one or more heteroatom(s) preferably selected from N, O and S, such as pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, pyranyl, carbazolyl, acridinyl, quinolinyl, benzimidazolyl, benzthiazolyl, purinyl, cinnolinyl and pteridinyl.

As used herein, the term “non-aromatic heterocycle” means a non-aromatic cyclic group containing one or more heteroatom(s) preferably selected from N, O and S, such as a pyrrolidinyl, piperidyl, piperazinyl, morpholinyl, tetrahydrofuranyl or monosaccharide.

As used herein, the term “halogen” means a fluorine, chlorine, bromine or iodine.

As used herein, the term “halo” means a fluoro, chloro, bromo or iodo.

As used herein, and unless specified otherwise, the term “substituted” means that the concerned groups are substituted with at least one functional group, such as hydroxyl, amine, sulfide, silyl, carboxylic acid, halogen, aryl, etc.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B. The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “daily” is intended to mean that a therapeutic compound is administered once or more than once each day, for example, for a period of time. The term “continuous” is intended to mean that a therapeutic compound is administered daily for an uninterrupted period of, e.g., at least 10 days. The term “intermittent” or “intermittently” as used herein is intended to mean stopping and starting at either regular or irregular intervals. For example, intermittent administration of the compound is administration for one to six days per week, administration in cycles (e.g., daily administration for two to eight consecutive weeks, then a rest period with no administration for up to one week), or administration on alternate days.

6.2 Agents for Combination Therapies
6.2.1 P53 Reactivators

One therapeutic agent used in the combination therapies described herein (e.g., in Section 6.4) is a p53 agonist such a p53 reactivator. In some embodiments, the p53 reactivator provided herein increases by at least 5%, at least 10%, at least 15%, at least 20%, at least 30%, at least 40%, at least 50%, at least 60%, at least 70%, at least 80%, or at least 90% of p53 activity, e.g., in a cell such as an immune cell. In a specific embodiment, the p53 reactivator provided herein increases by about 50% of p53 activity, e.g., in a cell such as an immune cell. The methods for measuring p53 activity and increase thereof are well known in the art. For example, in some embodiments, the increased activity corresponds to an increased level of transcription of p53 target genes, which may be studied by quantifying mRNA increase. An increased level of mRNA may be determined by methods known in the art. In some embodiments, mRNA is quantified by TaqMan analysis.

In some embodiments, the agonist of p53 provided herein increases wild type p53 activity. In some embodiments, the agonist of p53 provided herein is a p53 reactivator capable of reactivating a mutant p53. In some embodiments, the agonist of p53 provided herein is capable of increasing wild type p53 activity and capable of reactivating a mutant p53.

One therapeutic agent in the present combination therapies is a p53 reactivator. The p53 gene TP53 is a very common target for mutations in tumors. About half of all human tumors carry mutations in TP53. p53 halts the cell cycle and/or triggers apoptosis in response to various stress stimuli, including DNA damage, hypoxia, and oncogene activation (Ko, L. J. & Prives, C., Genes Dev. 10, 1054-1072 (1996); Sherr, C. J., Genes Dev. 12, 2984-2991 (1998)). Upon activation, p53 initiates the p53-dependent biological responses through transcriptional transactivation of specific target genes carrying p53 DNA binding motifs.

Analyses of a large number of mutant p53 genes in human tumors have revealed a strong selection for mutations that inactivate the DNA binding function of p53; most mutations in tumors are point mutations clustered in the part encoding the core domain of p53 (residues 94-292) that harbors the DNA binding activity (Béroud, C. & Soussi, T., Nucl. Acids Res. 26, 200-204 (1998)).

Both p53-induced cell cycle arrest and apoptosis could be involved in p53-mediated tumor suppression. While p53-induced cell cycle arrest could conceivably be reversed in different ways, p53-induced cell death would have advantage of being irreversible. There is indeed evidence from animal in vivo models (Symonds et al., Cell 78, 703-711 (1994)) and human tumors (Bardeesy et al., Cancer Res. 55, 215-219 (1995)) indicating that p53-dependent apoptosis plays a major role in the elimination of emerging tumors, particularly in response to oncogenic signaling. Moreover, the ability of p53 to induce apoptosis often determines the efficacy of cancer therapy (Lowe et al., Science 266, 807-810 (1994)).

In addition to hyperproliferative diseases, such as cancer, it is also known in the art that deficient p53 function is involved in a number of other disease states, e.g. autoimmune diseases and cardiac diseases.

For example, as shown in Mountz et al., Immunology, 6: 27-37 (1994), human autoimmune diseases share the common feature of an imbalance between the production and destruction of various cell types including lymphocytes (SLE), synovial cells (RA), and fibroblasts (scleroderma). Genes including TP53 that regulates apoptosis are also expressed abnormally. According to the authors, specific therapies that induce apoptosis without incurring side effects should improve treatment of autoimmune disease.

For another example, Bonafe et al., Cell Death and Differentiation, 11: 962-973 (2004) suggests that TP53 codon 72 polymorphism contributes to a genetically determined variability in apoptotic susceptibility among old people, which has a potentially relevant role in the context of an age-related pathologic condition, such as myocardial ischaemia.

Okuda et al., Journal of Neuroimmunology, 135: 29-37 (2003) suggests that p53 may be involved in the regulatory process of experimental autoimmune encephalomyelitis (EAE) through the control of cytokine production and/or the apoptotic elimination of inflammatory cells. EAE as a model for autoimmune inflammatory diseases of the central nervous system (CNS) is a widely used model for the human disease multiple sclerosis.

These results suggest that pharmacological restoration of p53 function would be beneficial in a number of disorders and diseases.

In some embodiments of the combination therapies provided herein, the p53 reactivator directly or indirectly targets a mutant p53 protein.

In some embodiments, the mutant TP53 includes a missense mutation, which is a point mutation in which a single nucleotide change results in a codon that codes for a different amino acid. Missense mutant p53 proteins can be broadly classified as DNA-contact mutants and structural mutants. p53 DNA contact mutant contains mutations present on amino acids directly binding to DNA, such as in mutants carrying single amino acid changes R248Q, R248W, R273H, and R273C, where R248Q denotes that the wild-type residue arginine in position 248 has been replaced by a glutamine. p53 structural mutants have an amino acid replacement that alters the overall architecture and/or the stability to abolish its DNA-binding ability, as reported in mutants carrying the R175H, Y220C, G245S, R249S, and R282W mutations.

In other embodiments, the mutant TP53 includes a nonsense mutation. A nonsense mutation is a genetic mutation changing a codon for an amino acid into a stop codon, resulting in a shorter, unfinished protein product. Nonsense mutations are less frequent than missense mutations in TP53, but nonetheless constitute about 10% of all TP53 mutations in cancer. The most common TP53 nonsense mutation yields a truncated p53; R213X aka R213*.

In some embodiments, the p53 reactivator provided herein reactivates or restores at least part of the wild-type p53 activity of a mutant p53, for example by promoting proper folding of the mutant p53 and restoring the normal p53 function of the mutant p53.

In some more specific embodiments, the p53 reactivator provided herein or a degradation product or metabolite thereof inhibits improper protein misfolding and/or promotes proper protein folding by covalent binding to the mutant p53 protein, for example, by electrophiles binding to one or more thiols in the mutant p53 DNA binding domain to stabilize mutant p53 conformation, thus restoring their transcriptional activities. In some embodiments, the p53 reactivator provided herein or a degradation product or metabolite thereof binds to the thiol of cysteine residues in the core domain and stabilizes wild type p53 conformation. In other embodiments, the p53 reactivator provided herein or a degradation product or metabolite thereof is shifting the equilibrium from unfolded towards a wild-type like p53 conformation. In yet other embodiments, the p53 reactivator provided herein or a degradation product or metabolite thereof binds to thiol groups in the core domain and restores wild-type conformation. In other more specific embodiments, the p53 reactivator provided herein or a degradation product or metabolite thereof inhibits improper protein misfolding and/or promotes proper protein folding by non-covalent binding to the mutant p53 protein. Such p53 reactivators include chaperones that can non-covalently stabilize mutant p53 structures.

In certain embodiments, the p53 reactivator provided herein inhibits improper protein misfolding of the mutant p53 protein, and/or promotes proper protein folding of the mutant p53 by covalently binding to the mutant p53 protein.

In certain embodiment, the p53 reactivator (or a metabolite or degradation product of the p53 reactivator) provided herein reacts as an electrophile with one or more thiols in the mutant p53. In certain embodiment, the p53 reactivator (or a metabolite or degradation product of the p53 reactivator) provided herein covalently binds to one or more thiols in the mutant p53. In certain embodiment, the p53 reactivator (or a metabolite or degradation product of the p53 reactivator) reacts with or binds to one or more thiols in the mutant p53 as an electrophile. In certain embodiments, a metabolite or degradation product of the p53 reactivator) reacts with or binds to one or more thiols in the mutant p53. In certain embodiments, reversible or irreversible covalent bonds are formed between the p53 reactivator (or a metabolite or degradation product of the p53 reactivator) and the mutant p53. In one embodiment, reversible covalent bonds are formed between the p53 reactivator (or a metabolite or degradation product of the p53 reactivator) and the mutant p53. In another embodiment, irreversible covalent bonds are formed between the p53 reactivator (or a metabolite or degradation product of the p53 reactivator) and the mutant p53.

In certain embodiments, the p53 reactivator (or a metabolite or degradation product of the p53 reactivator) provided herein reacts with one or more thiols in the DNA binding domain of the mutant p53 to stabilize the mutant p53 conformation, thus restoring their transcriptional activities. In some embodiments, the p53 reactivator (or a metabolite or degradation product of the p53 reactivator) reacts with one or more thiols of cysteine residues in the core domain of wild-type p53 protein and stabilizes wild-type p53 conformation. In other embodiments, the p53 reactivator (or a metabolite or degradation product of the p53 reactivator) reacts with one or more thiols of cysteine residues in the core domain of mutant p53 and restores the mutant p53 conformation to wild-type p53 like conformation. In yet other embodiments, the p53 reactivator (or a metabolite or degradation product of the p53 reactivator) shifts the equilibrium from unfolded mutant p53 conformation towards a wild-type p53 like conformation.

In other embodiments, the p53 reactivator provided herein or a degradation product or metabolite thereof reactivates mutant p53 by interfering with aggregation of misfolded p53 or reducing aggregation of mutant p53. Sometimes, p53 misfolds or unfolds into an aggregation-prone stage that loses its DNA-binding capacity. Similarly, misfolded p53 may convert wild type p53 to a misfolded form and accelerate p53 aggregation. Thus, in some embodiments, the p53 reactivator provided herein or a degradation product or metabolite thereof may reactivate p53 by interfering with aggregation of misfolded p53. In other embodiments, the p53 reactivator provided herein or a degradation product of metabolite reduces non-folded or incorrectly folded mutant p53 that may otherwise aggregate, and thereby reducing aggregation.

In certain embodiments, the p53 reactivator provided herein reactivates mutant p53 by interfering with aggregation of misfolded p53 or reducing aggregation of mutant p53. Sometimes, p53 misfolds or unfolds into an aggregation-prone stage that loses its DNA-binding capacity. Similarly, misfolded p53 may convert wild type p53 to a misfolded form and accelerate p53 aggregation. Thus, in some embodiments, the p53 reactivator provided herein may reactivate p53 by interfering with aggregation of misfolded p53. In other embodiments, the p53 reactivator provided herein or a degradation product of metabolite reduces non-folded or incorrectly folded mutant p53 that may otherwise aggregate, and thereby reducing aggregation.

In some embodiments, the mutant p53 contains at least one replacement in the core domain of p53 (residues 94-292) caused by a TP53 mutation. In some embodiments, the mutant p53 contains at least one of the following amino acid replacements: V173A, S241F, R249S, R273H, R175H, R248Q, and Y220C.

In some embodiments, the mutant p53 comprises at least one amino acid replacement in the core domain of the mutant p53 (between residues 94 and 292) caused by a TP53 mutation. In some embodiments, the mutant p53 comprises one or more of the amino acid replacements selected from the group consisting of V173A, S241F, R249S, R273H, R175H, R248Q, and Y220C. In some embodiments, the mutant p53 comprises one of the amino acid replacements of R175H and R273H.

In some embodiments, the p53 reactivator or a degradation product or metabolite thereof may enhance the activity of wild type p53, directly or indirectly. As shown in Section 7 below, surprisingly, the combination of APR-246 and ABT-199 also generates synergistic effects in cancer with wild type p53. Without being bound by any theory, certain p53 reactivator provided herein may activate wild type p53 as well by direct binding to thiols in the DNA binding domain, as outlined in detail for mutant p53 above. In addition, in some embodiments, the p53 reactivator or a degradation product or metabolite thereof may have additional cellular targets that reinforce its cell-death inducing effect and allows pharmacologically relevant activity in cells with wild type TP53 or devoid of TP53 or producing a truncated p53 protein. For APR-246, such targets may include glutathione, thioredoxin reductase 1, thioredoxin 1, glutaredoxin 1 and ribonucleotide reductase, resulting in increased cellular oxidative stress and thus increased propensity for cell death. Other possible targets may be found, for example, in Bykov et al., Front Oncol., 6:21 (2016) and Haffo et al., Sci Rep., 8(1):12671 (2018). Thus, in some embodiments, the combination treatment provided herein can be used to treat cancer having wild-type p53.

In certain embodiments, the p53 reactivator provided herein can give reactivation of a mutant p53 protein. In certain embodiments, the p53 reactivator can result in reactivation of a mutant p53 protein. In certain embodiments, the p53 reactivator is transformed to a metabolite or a degradation product that reacts with a mutant p53 protein. In certain embodiments, the p53 reactivator is transformed to the metabolite or the degradation product in vivo. In certain embodiments, the p53 reactivator is transformed to the metabolite or the degradation product in tumor tissue.

In addition, in another aspect of the present disclosure, the p53 reactivator provided herein can be used as a monotherapy to treat a hyperproliferative malignancy that does not comprise a cancer cell having mutant p53 or a hyperproliferative malignancy comprising a cancer cell having wild type p53. In a specific embodiment, the p53 reactivator is APR-246. In another specific embodiment, the p53 reactivator is Compound A.

In yet another specific embodiment, the p53 reactivator is Compound B. In yet another specific embodiment, the p53 reactivator is Compound C. In yet another specific embodiment, the p53 reactivator is Compound D. In yet another specific embodiment, the p53 reactivator is Compound E.

Without being bound by any theory, in some embodiments, the p53 reactivator provided herein or a degradation product or metabolite stabilizes wild type p53 protein in a situation where its production has been induced as part of a normal physiological process, and in this way enhancing the effect of said wild type p53 induction.

In some embodiments, the p53 reactivator provided herein is a compound according to formula (I)

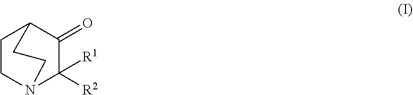

wherein:
R¹ is selected from H, —CH₂—O—R³, —CH₂—S—R³, and —CH₂—NR³R⁴;
R² is selected from —CH₂—O—R³, —CH₂—S—R³, and —CH₂—NR³R⁴;
R³ and R⁴ are the same or different and are independently selected from H; substituted or non-substituted, unbranched or branched, saturated or unsaturated C3-C12 cycloalkyl or C1-C10 alkyl; substituted or non-substituted benzyl; substituted or non-substituted mono- or bicyclic aryl; substituted or non-substituted mono-, bi- or tricyclic C2-C10 heteroaryl or non-aromatic C2-C10 heterocyclyl containing one or several heteroatoms independently selected from N, O and S; or R³ and R⁴ in —CH₂—NR³R⁴ are bonded together and form, together with the nitrogen atom to which they are bonded, a substituted or non-substituted non-aromatic C2-C10 mono- or bicyclic heterocyclyl optionally containing one or several further heteroatoms independently selected from N, O and S and optionally comprising one or several cyclic keto groups;
wherein the substituents of the substituted groups are independently selected from unbranched or branched, saturated or unsaturated C3-C12 cycloalkyl or C1-C10 alkyl; halogen; halogen-substituted C1-C10 alkyl, mono- or bicyclic aryl; mono-, bi- or tricyclic C2-C10 heteroaryl or non-aromatic C2-C10 heterocyclyl containing one or several heteroatoms independently selected from N, O and S; C1-C10 alkoxy; amino; and C1-C10 alkylamino;
or a pharmaceutically acceptable salts thereof.

In certain embodiments, the compound that can give reactivation of mutant p53 (the p53 reactivator) provided herein is a compound according to formula (I):

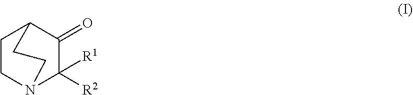

wherein:
R¹ is selected from the group consisting of H, —CH₂—O—R³, —CH₂—S—R³, and —CH₂—NR³R⁴;
R² is selected from the group consisting of —CH₂—O—R³, —CH₂—S—R³, and CH₂—NR³R⁴;
each of R³ and R⁴ is independently selected from H; substituted or unsubstituted, unbranched or branched, saturated or unsaturated C3-C12 cycloalkyl or C1-C10 alkyl; substituted or unsubstituted benzyl; substituted or unsubstituted mono- or bi-cyclic aryl; substituted or unsubstituted mono-, bi- or tri-cyclic C3-C10 heteroaryl or non-aromatic C3-C10 heterocyclyl containing one or more heteroatoms independently selected from N, O and S; or R³ and R⁴ in —CH₂—NR³R⁴ together with the nitrogen atom to which they are attached, form a substituted or unsubstituted non-aromatic C3-C10 mono- or bi-cyclic heterocyclyl containing one or more heteroatoms independently selected from N, O and S and optionally comprising one or more keto substitute groups;
wherein the substituents of the substituted groups are independently selected from unbranched or branched, saturated or unsaturated C3-C12 cycloalkyl or C1-C10 alkyl; halogen; halo-substituted C1-C10 alkyl, mono- or bicyclic aryl; mono-, bi- or tricyclic C3-C10 heteroaryl or non-aromatic C2-C10 heterocyclyl containing one or more heteroatoms independently selected from N, O and S; C1-C10 alkoxy; amino; and C1-C10 alkylamino;
   or a pharmaceutically acceptable salt thereof.

The pharmaceutically acceptable salt of the compound of formula (I) e.g. may be an acid addition salt of an inorganic mineral acid or of an organic acid.

The pharmaceutically acceptable salt of the compound of formula (I) may be an acid addition salt of the compound formed with an inorganic acid or of an organic acid.

In a compound of formula (I), R¹ is selected from H, —CH₂—O—R³, —CH₂—S—R³, and —CH₂—NR³R⁴.

In some embodiments, R¹ is H. or —CH₂—O—R³. In some embodiments, R¹ is—CH₂—O—R³ or —CH₂—S—R³. In some embodiments, R¹ is H.

In some embodiments, R¹ is selected from H, —CH₂—O—R³, and —CH₂—S—R³. In some embodiments, R¹ is selected from H and —CH₂—O—R³. In other embodiments, R¹ is selected from —CH₂—O—R³, and —CH₂—S—R³. In some embodiments, R¹ is H.

R² in formula (I) is selected from —CH₂—O—R³, —CH₂—S—R³, and —CH₂—NR³R⁴. In some embodiments, R² is selected from —CH₂—O—R³ and —CH₂—S—R³. In still other embodiments, R² is —CH₂—O—R³.

In one embodiment, R¹ is selected from H, —CH₂—O—R³ and —CH₂—S—R³; and R³ is selected from —CH₂—O—R³ and —CH₂—S—R³.

In one embodiment, R¹ is H; and R² is selected from —CH₂—O—R³, —CH₂—S—R³ and —CH₂—NR³R⁴; e.g. from —CH₂—O—R³ and —CH₂—S—R³, and in particular is CH₂—O—R³.

In another embodiment, R¹ is H, and R² is —CH₂—O—R³ or —CH₂—S—R³. In yet another embodiment, R¹ is H, and R² is —CH₂—O—R³.

In one embodiment, R¹ is selected from H and —CH₂—O—R³; and R² is —CH₂—O—R³.

In one embodiment, both R¹ and R² are —CH₂—O—R³.

In one embodiment, each R³ is independently selected from H; substituted or non-substituted, unbranched or branched, saturated or unsaturated C3-C12 cycloalkyl and C1-C10 alkyl, and benzyl. For example, each R³ may be independently selected from H and C1-C10 alkyl, e.g. from H and C1-C6 alkyl, from H and C1-C4 alkyl, or from H and C1-C3 alkyl, in particular from H and methyl.

In one embodiment, R¹ is selected from H and —CH₂—O—R³, and R² is —CH₂—O—R³, and each R³ is independently selected from H; substituted or non-substituted, unbranched or branched, saturated or unsaturated C3-C12 cycloalkyl and C1-C10 alkyl, and benzyl, in particular from H and C1-C10 alkyl, e.g. from H and C1-C6 alkyl, from H and C1-C4 alkyl, or from H and C1-C3 alkyl, in particular from H and methyl.

In one embodiment, R¹ and R² are both —CH₂—O—R³, and each R³ is independently selected from H; substituted or non-substituted, unbranched or branched, saturated or unsaturated C3-C12 cycloalkyl and C1-C10 alkyl; in particular from H and C1-C10 alkyl; e.g. from H and C1-C6 alkyl, from H and C1-C4 alkyl, or from H and C1-C3 alkyl, in particular from H and methyl.

In one embodiment, R¹ is H or —CH₂—O—R³, R² is —CH₂—O—R³, and each R³ is independently selected from H; substituted or non-substituted, unbranched or branched, saturated or unsaturated C3-C12 cycloalkyl and C1-C10 alkyl, and benzyl. In another embodiment, R¹ is H or —CH₂—O—R³, R² is —CH₂—O—R³, and each R³ is independently H or C1-C10 alkyl. In yet another embodiment, R¹ is H or —CH₂—O—R³, R² is —CH₂—O—R³, and each R³ is independently H or C1-C6 alkyl. In yet another embodiment, R¹ is H or —CH₂—O—R³, R² is —CH₂—O—R³, and each R³ is independently H or C1-C4 alkyl. In yet another embodiment, R¹ is H or —CH₂—O—R³, R² is —CH₂—O—R³, and each R³ is independently H or C1-C3 alkyl. In a specific embodiment, R¹ is H or —CH₂—O—R³, R² is —CH₂—O—R³, and each R³ is independently H or methyl.

In one embodiment, R¹ and R² are both —CH₂—O—R³, and each R³ is independently selected from H; substituted or non-substituted, unbranched or branched, saturated or unsaturated C3-C12 cycloalkyl and C1-C10 alkyl. In another embodiment, R¹ and R² are both —CH₂—O—R³, and each R³ is independently H or C1-C10 alkyl. In yet another embodiment, R¹ and R² are both —CH₂—O—R³, and each R³ is independently H or C1-C6 alkyl. In yet another embodiment, R¹ and R² are both —CH₂—O—R³, and each R³ is independently H or C1-C4 alkyl. In yet another embodiment, R¹ and R² are both —CH₂—O—R³, and each R³ is independently H or C1-C3 alkyl. In a specific embodiment, R¹ and R² are both —CH₂—O—R³, and each R³ is independently H or methyl.

In a compound of formula (I), as defined herein above, any C1-C10 alkyl e.g. may be a C1-C6 alkyl, or a C1-C4 alkyl, e.g. methyl, ethyl, propyl or butyl. Any C3-C12 cycloalkyl may be e.g. a C3-C8 cycloalkyl, or a C3-C6 cycloalkyl. Any mono- or bicyclic aryl may be e.g. a monocyclic aryl, such as phenyl. Any mono-, bi- or tricyclic C2-C10 heteroaryl may be e.g. a monocyclic or bicyclic C2-C5 heteroaryl, e.g. a 5- or 6-membered monocyclic or a 9-membered bicyclic C2-C5 heteroaryl. Any mono-, bi- or tricyclic non-aromatic C2-C10 heterocyclyl may be e.g. a monocyclic or bicyclic C2-C5 heterocyclyl, e.g. a 5- or 6-membered monocyclic or 9- or 10-membered bicyclic C2-C5 heterocyclyl. Any halogen may be selected from F, Cl, Br and I, preferably from F and Cl. Any heterocycle, aromatic or not, containing one or several heteroatoms independently selected from N, O and S, e.g. may contain 1-5 heteroatoms, e.g. independently selected from N and O.

In one embodiment, in a compound of formula (I) as defined herein above, any substituted or non-substituted C3-C12 cycloalkyl or C1-C10 alkyl is non-substituted.

In one embodiment, any substituted or non-substituted benzyl is non-substituted.

In one embodiment, any substituted or non-substituted mono- or bicyclic aryl is non-substituted.

In one embodiment, any substituted or non-substituted mono-, bi- or tricyclic C2-C10 heteroaryl or non-aromatic C2-C10 heterocyclyl is non-substituted.

In one embodiment, when any of the above groups is substituted, each substituent is selected from C1-C10 alkyl, e.g. C1-C6 alkyl, C1-C4 alkyl, or C1-C3 alkyl, such as methyl; halogen, e.g. Cl; halogen-substituted C1-C10 alkyl, e.g. trifluoromethyl; monocyclic C2-C5 heteroaryl, e.g. pyridyl; C1-C10 alkoxy, e.g. C1-C6 alkoxy, C1-C4 alkoxy, or C1-C3 alkoxy, such as methoxy; and amino.

In one embodiment of the compound of formula (I) as defined herein above, the substituted or unsubstituted C3-C12 cycloalkyl or C1-C10 alkyl is a non-substituted C3-C12 cycloalkyl or C1-C10 alkyl.

In one embodiment, the substituted or unsubstituted benzyl is an unsubstituted benzyl.

In one embodiment, the substituted or unsubstituted mono- or bi-cyclic aryl is an unsubstituted mono- or bi-cyclic aryl.

In one embodiment, the substituted or unsubstituted mono-, bi- or tri-cyclic heteroaryl or non-aromatic heterocyclyl is an unsubstituted mono-, bi- or tri-cyclic heteroaryl or non-aromatic heterocyclyl.

In one embodiment, when any of the above groups is substituted, each substituent independently is a C1-C10 alkyl, halo, halo-substituted C1-C10 alkyl, monocyclic heteroaryl, C1-C10 alkoxy, or amino group.

In one embodiment, when any of the above groups is substituted, the number of substituents on each substituted group is 1, 2 or 3.

In another embodiment, the compound provided herein is selected from those exemplified in the prior art documents referred to herein above, e.g. WO05/090341, WO04/084893, WO02/024692 and WO03/070250.

In one embodiment, the compound of formula (I) is selected from 2-(hydroxymethyl)-2-(methoxymethyl)quinuclidin-3-one and 2,2-bis(hydroxymethyl)quinuclidin-3-one, and pharmaceutically acceptable salts of these compounds.

In one embodiment, the compound of formula (I) is 2-(hydroxymethyl)-2-(methoxymethyl)quinuclidin-3-one (APR-246) or a pharmaceutically acceptable salt thereof.

In one embodiment, the compound of formula (I) is 2-(hydroxymethyl)-2-(methoxymethyl)quinuclidin-3-one (APR-246) or 2,2-bis(hydroxymethyl)quinuclidin-3-one, or a pharmaceutically acceptable salt thereof.

In one embodiment, the compound of formula (I) is 2-(hydroxymethyl)-2-(methoxymethyl)quinuclidin-3-one (APR-246) having the following formula:

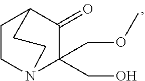

or a pharmaceutically acceptable salt thereof.

In another embodiment, the compound of formula (I) is 2,2-bis(hydroxymethyl)quinuclidin-3-one or a pharmaceutically acceptable salt thereof.

In yet other embodiments, the p53 reactivator provided herein is selected from the group consisting of: 2,2,2-trichloro-N-ethyl-N-((3-oxoquinuclidin-2-yl)methyl)acetamide; 2,2,2-trichloro-N-((3-oxoquinuclidin-2-yl)methyl)acetamide; N-ethyl-2,2,2-trifluoro-N-((3-oxoquinuclidin-2-yl)methyl)acetamide; 2,2,2-trifluoro-N-((3-oxoquinuclidin-2-yl)methyl)acetamide; 2,2-difluoro-N-((3-oxoquinuclidin-2-yl)methyl)acetamide, N-((3-oxoquinuclidin-2-yl)methyl)pyridine-3-sulfonamide; 4-fluoro-N-((3-oxoquinuclidin-2-yl)methyl)benzenesulfonamide; N-ethyl-N-((3-oxoquinuclidin-2-yl)methyl)methanesulfonamide; N-((3-oxoquinuclidin-2-yl)methyl)methanesulfonamide; N-((3-oxoquinuclidin-2-yl)methyl)benzenesulfonamide; 2-(N-((3-oxoquinuclidin-2-yl)methyl)methylsulfonamido)acetamide; N-(methylsulfonyl)-N-((3-oxoquinuclidin-2-yl)methyl)glycine; N-((3-oxoquinuclidin-2-yl)methyl)pyridine-4-sulfonamide; N-((3-oxoquinuclidin-2-yl)methyl)pyridine-2-sulfonamide; N-ethyl-1,1,1-trifluoro-N-((3-oxoquinuclidin-2-yl)-methyl)methanesulfonamide; 1,1,1-trifluoro-N-((3-oxoquinuclidin-2-yl)methyl)methanesulfonamide; N,N-bis((3-oxoquinuclidin-2-yl)methyl)methanesulfonamide; N-((3-oxoquinuclidin-2-yl)methyl)propane-2-sulfonamide; N-((3-oxoquinuclidin-2-yl)methyl)cyclopropanesulfonamide; 1-methyl-N-((3-oxoquinuclidin-2-yl)methyl)cyclopropane-1-sulfonamide; N-cyclopropyl-N-((3-oxoquinuclidin-2-yl)methyl)methanesulfonamide; N-((3-oxoquinuclidin-2-yl)methyl)-N-phenylmethanesulfonamide; 1-((3-oxoquinuclidin-2-yl)methyl)pyrimidine-2,4(1H,3H)-dione; 5-methyl-1-((3-oxoquinuclidin-2-yl)methyl)pyrimidine-2,4(1H,3H)-dione; tert-butyl 5-methyl-2,6-dioxo-3-((3-oxoquinuclidin-2-yl)methyl)-3,6-dihydropyrimidine-1(2H)-carboxylate; 5-methyl-1,3-bis((3-oxoquinuclidin-2-yl)methyl)pyrimidine-2,4(1H,3H)-dione; N-methyl-1-((3-oxoquinuclidin-2-yl)methyl)-1H-1,2,4-triazole-3-carboxamide; 2-((3-chloro-1H-1,2,4-triazol-1-yl)methyl)quinuclidin-3-one; N,N-dimethyl-1-((3-oxoquinuclidin-2-yl)methyl)-1H-1,2,4-triazole-3-carboxamide; 2-((1H-1,2,4-triazol-1-yl)methyl)quinuclidin-3-one; 1-((3-oxoquinuclidin-2-yl)methyl)-1H-1,2,4-triazole-3-carbonitrile; and 1-((3-oxoquinuclidin-2-yl)methyl)-1H-1,2,4-triazole-3-carboxamide, or a pharmaceutically acceptable salt thereof.

In yet another embodiment, the compound of formula (I) is a compound listed in Table 1 below, or a pharmaceutically acceptable salt thereof.

TABLE 1

| Compound | Name | Structure |
|---|---|---|
| Compound A | 2,2,2-trifluoro-N- ((3-oxoquinuclidin- 2-yl) methyl)acetamide | 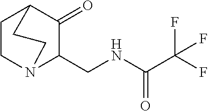 [SMILES: O=C1C2CCN(CC2)C1CNC(=O)C(F)(F)F] |
| Compound B | 2,2,2-trichloro- N-ethyl-N- ((3-oxoquinuclidin- 2- yl)methyl) acetamide | 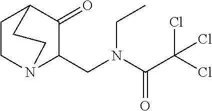 [SMILES: CCN(CC1C(=O)C2CCN1CC2)C(=O)C(Cl)(Cl)Cl] |
| Compound C | 2,2,2-trichloro-N- ((3-oxoquinuclidin- 2-yl)methyl) acetamide | 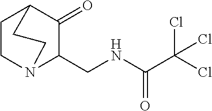 [SMILES: O=C1C2CCN(CC2)C1CNC(=O)C(Cl)(Cl)Cl] |
| Compound D | N-ethyl-2,2,2- trifluoro-N-((3- oxoquinuclidin-2- yl)methyl) acetamide | 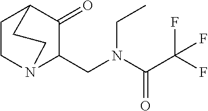 [SMILES: CCN(CC1C(=O)C2CCN1CC2)C(=O)C(F)(F)F] |
| Compound E | 2,2-difluoro-N- ((3-oxoquinuclidin- 2-yl)methyl) acetamide | 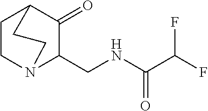 [SMILES: O=C(NCC1C(=O)C2CCN1CC2)C(F)F] |

In yet another embodiment, the p53 reactivator is Compound A, or a pharmaceutically acceptable salt thereof. In yet another embodiment, the p53 reactivator is Compound B, or a pharmaceutically acceptable salt thereof. In yet another embodiment, the p53 reactivator is Compound C, or a pharmaceutically acceptable salt thereof. In yet another embodiment, the p53 reactivator is Compound D, or a pharmaceutically acceptable salt thereof. In yet another embodiment, the p53 reactivator is Compound E, or a pharmaceutically acceptable salt thereof.

In a specific embodiment, the p53 reactivator provided herein is 2,2,2-trifluoro-N-((3-oxoquinuclidin-2-yl)methyl)acetamide (Compound A) having the following formula:

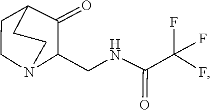 [SMILES: O=C1C2CCN(CC2)C1CNC(=O)C(F)(F)F]

or a pharmaceutically acceptable salt thereof.

In another specific embodiment, the p53 reactivator provided herein is 2,2,2-trichloro-N-ethyl-N-((3-oxoquinuclidin-2-yl)methyl)acetamide (Compound B) having the following formula:

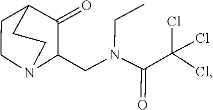 [SMILES: CCN(CC1C(=O)C2CCN1CC2)C(=O)C(Cl)(Cl)Cl]

or a pharmaceutically acceptable salt thereof.

In yet another specific embodiment, the p53 reactivator provided herein is 2,2,2-trichloro-N-((3-oxoquinuclidin-2-yl)methyl)acetamide (Compound C) having the following formula:

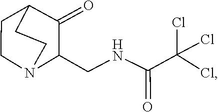 [SMILES: O=C1C2CCN(CC2)C1CNC(=O)C(Cl)(Cl)Cl]

or a pharmaceutically acceptable salt thereof.

In yet another specific embodiment, the p53 reactivator provided herein is N-ethyl-2,2,2-trifluoro-N-((3-oxoquinuclidin-2-yl)methyl)acetamide (Compound D) having the following formula:

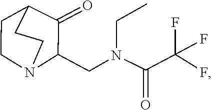 [SMILES: CCN(CC1C(=O)C2CCN1CC2)C(=O)C(F)(F)F]

or a pharmaceutically acceptable salt thereof.

In yet another specific embodiment, the p53 reactivator provided herein is 2,2-difluoro-N-((3-oxoquinuclidin-2-yl)methyl)acetamide (Compound E) having the following formula:

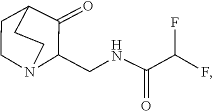 [SMILES: O=C(NCC1C(=O)C2CCN1CC2)C(F)F]

or a pharmaceutically acceptable salt thereof.

In some embodiments, the p53 reactivator is a compound listed in Table 2 below. In certain more specific embodiments, the compound listed in the table below is capable of reactivating mutant p53.

TABLE 2

| Compound | |
|---|---|
| Name | Structure |
| CP31398 | 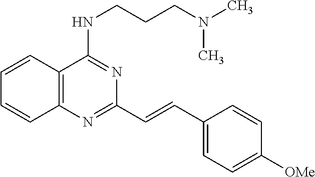 [SMILES: COc1ccc(/C=C/c2nc(NCCCN(C)C)c3ccccc3n2)cc1] |
| MIRA-1 | 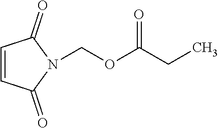 |
| PK11007 | 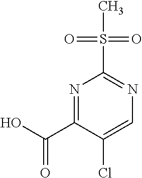 |
| SCH529074 | 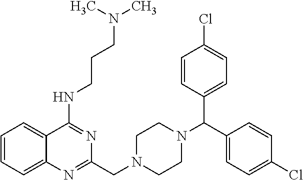 [SMILES: CN(C)CCCNc1nc(CN2CCN(C(c3ccc(Cl)cc3)c3ccc(Cl)cc3)CC2)nc2ccccc12] |
| P53R3 | 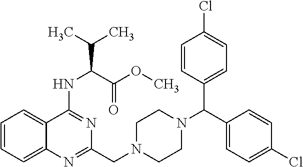 [SMILES: COC(=O)[C@@H](Nc1nc(CN2CCN(C(c3ccc(Cl)cc3)c3ccc(Cl)cc3)CC2)nc2ccccc12)C(C)C] |
| Stitic acid | 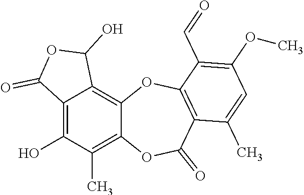 [SMILES: COc1cc(C)c2c(c1C=O)Oc1c(c(C)c(O)c3c1C(O)OC3=O)OC2=O] |
| PK7088 | 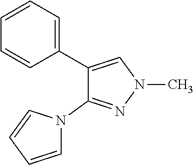 [SMILES: Cn1cc(-c2ccccc2)c(-n2cccc2)n1] |
| PhiKan083 | 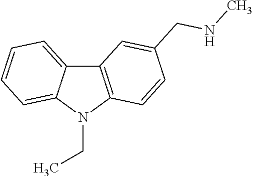 [SMILES: CCn1c2ccccc2c2cc(CNC)ccc21] |
| R-GON | 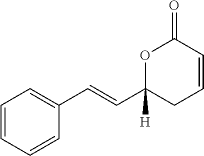 |
| MB725 | 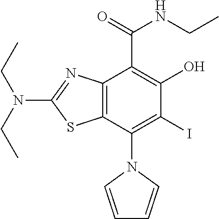 [SMILES: CCNC(=O)c1c(O)c(I)c(-n2cccc2)c2sc(N(CC)CC)nc12] |
| NSC319726 | 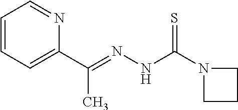 [SMILES: C/C(=N\NC(=S)N1CCC1)c1ccccn1] |
| COTI-2 | 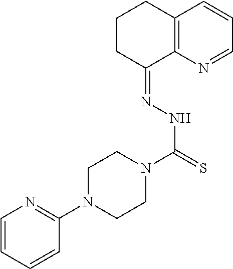 [SMILES: S=C(N/N=C1/CCCc2cccnc21)N1CCN(c2ccccn2)CC1] |
| PEITC | 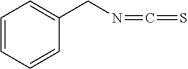 |
| pCAP-250^{a} | Myr-RRHSTPHPD |
| CDB3^{b} | REDEDEIEW |
| P53R175H- | ATTAGCGCATTTTAACATAGGGTGC |
| APT^{c} | |
| ReACp53^{b} | RRRRRRRRRLTRITLE |
| SAHA | 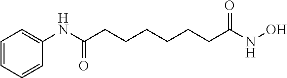 [SMILES: O=C(CCCCCCC(=O)Nc1ccccc1)NO] |
| Chetomin | 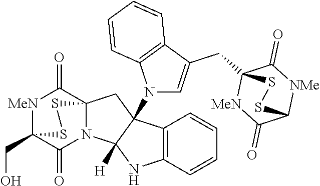 |
| KSS-9 | 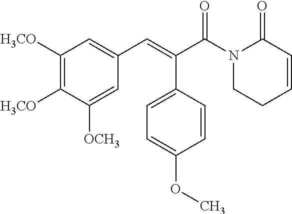 [SMILES: COc1ccc(/C(=C\c2cc(OC)c(OC)c(OC)c2)C(=O)N2CCC=CC2=O)cc1] |
| SLMP53-1 | 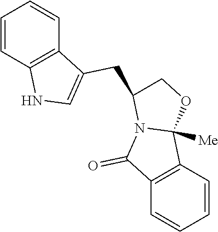 [SMILES: C[C@]12OC[C@H](Cc3c[nH]c4ccccc34)N1C(=O)c1ccccc12] |

6.2.2 Bcl-2 Inhibitors

The term “Bcl-2” as used herein refers to the Bcl-2 protein (Swiss Prot ID No. P10415), a member of the Bcl-2 family of proteins (Cory, S., and Adams, J. M., Nature Reviews Cancer, 2: 647-656 (2002); Petros. A. M., Biochim Biophys Acta, 1644: 83-94 (2004); Danial, N. N., and Korsmeyer. S. J., Cell, 116: 205-219 (2004)).

The Bcl-2 family of proteins regulates programmed cell death (Cory. S., and Adams, J. M., Nature Reviews Cancer, 2: 647-656 (2002); Adams, Genes and Development, 17: 2481-2495 (2003); Danial, N. N., and Korsmeyer, S. J., Cell, 116: 205-219 (2004)).

Cell survival is promoted by Bcl-2 itself and several close relatives (Bcl-xL, Bcl-W, Mcl-1 and A1), which bear three or four conserved Bcl-2 homology (BH) regions. These Bcl-2 family proteins are therefore also referred to as anti-apoptotic Bcl-2 proteins. In some embodiments, provided herein is combination therapies comprising an inhibitor of an anti-apoptotic Bcl-2 protein. In some embodiments, the present combination therapies comprise an inhibitor of Bcl-2 (or a Bcl-2 inhibitor). In some embodiments, the present combination therapies comprise an inhibitor of Mcl-1. In some embodiments, the present combination therapies comprise an inhibitor of Bcl-xL. In some embodiments, the present combination therapies comprise an inhibitor of Bcl-W. In some embodiments, the present combination therapies comprise an inhibitor of A1.

Apoptosis is driven by two other sub-families, including BH3-only proteins such as Bad, Bid, Bim, Puma and Noxa, and multi-domain proteins containing BH1-BH3 such as Bax and Bak (see Cheng, et al., Molecular Cell, 8: 705-711 (2001); Wei, M. C., et al., Science, 292: 727-730 (2001); Zong, W. X., et al., Genes and Development, 15(148): 1-1486 (2001); Wang, K., Genes and Development, 15: 2922-2933 (2001); Huang and Strasser, Cell, 103: 839-842 (2003); Green, D. R., and Kroemer, G., Science, 305: 626-629 (2004)).

An enhanced level of anti-apoptotic Bcl-2 proteins are associated with a number of diseases. For example, Bcl-2 proteins have been shown to involve in bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, and other cancer, as described, e.g., in WO 2005/049593 and WO 2005/024636. Bcl-2 proteins have also been shown to involve in immune and autoimmune diseases, as described, e.g., in Current Allergy and Asthma Reports 2003, 3, 378-384; British Journal of Hematology 2000, 110(3), 584-90; and New England Journal of Medicine 2004, 351(14), 1409-1418.

There exist various inhibitors that inhibit prosurvival members of the Bcl-2 family of proteins and are therefore promising candidates for the treatment of cancer. Such inhibitors include, e.g., Oblimersen, SPC-2996, RTA-402, Gossypol, AT-101, Obatoclax mesylate, A-371191, A-385358, A-438744, ABT-737, ABT-263, AT-101, BL-11, BL-193, GX-15-003, 2-Methoxyantimycin A₃, HA-14-1. KF-67544, Purpurogallin, TP-TW-37, YC-137 and Z-24, and are described, e.g. in Zhai, D., et al., Cell Death and Differentiation, 13: 1419-1421 (2006).

Thus, in some embodiments, the inhibitor provided herein is selected from the group consisting of Oblimersen, SPC-2996, RTA-402, Gossypol, AT-101, Obatoclax mesylate, A-371191, A-385358, A-438744, ABT-737, ABT-263, AT-101, BL-11, BL-193, GX-15-003, 2-Methoxyantimycin A3, HA-14-1. KF-67544, Purpurogallin, TP-TW-37, YC-137 and Z-24.

In some embodiments, the Bcl-2 inhibitor provided herein is a selective inhibitor of Bcl-2, which exhibits greater affinity for Bcl-2 than Bcl-xL, such as venetoclax (or ABT-199). Owing to its high subnanomolar affinity for Bcl-2 and low binding to Bcl-xL, this class of Bcl-2 inhibitor may not cause thrombocytopenia.

In some embodiments, the Bcl-2 inhibitor provided herein is a Bcl-2 inhibitor described in U.S. Pat. No. 8,546,399, which disclosure is incorporated by reference herein.

In some embodiments, the Bcl-2 inhibitor provided herein is a Bcl-2 inhibitor described in WO2012/121758, which disclosure is incorporated by reference herein. More specifically, in some embodiments, the Bcl-2 inhibitor provided herein is a compound of the following formula (Formula (II)):

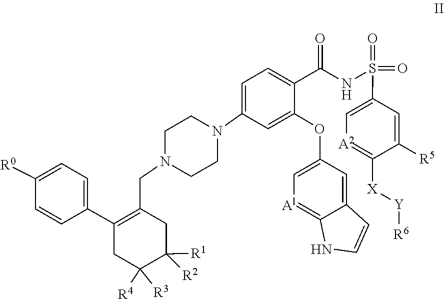

wherein:
R⁰ is halo;
R¹ and R² are H or are independently methyl or methoxy;
R³ and R⁴ are independently methyl or methoxy if R¹ and R² are H, or are H if R¹ and R² are independently methyl or methoxy;
A¹ and A² are each independently CH or N;
R⁵ is C₁₋₄ alkyl or haloalkyl, C₁₋₄ alkylsulfonyl or haloalkylsulfonyl, halo, nitro or cyano;
X is —O— or —NH—;
Y is —(CH₂)ₙ— where n is 0, 1, 2 or 3; and
R⁶ is an unsubstituted or substituted 3- to 7-membered carbocyclic or heterocyclic ring as defined herein, or is NR⁷R⁸;
wherein, if R⁶ is NR⁷R⁸, R⁷ and R⁸ are each independently H or R⁹—(CH₂)ₘ— groups, no more than one of R⁷ and R⁸ being H, where each R⁹ is independently a 3- to 7-membered carbocyclic or heterocyclic ring, optionally substituted with no more than two Z¹ groups as defined below, and each m is independently 0 or 1; and
wherein, if R⁶ is a substituted carbocyclic or heterocyclic ring, substituents thereon are no more than two Z¹ groups and/or no more than one Z² group, Z¹ groups being independently selected from the group consisting of (a) C₁₋₄ alkyl, C₂₋₄ alkenyl, C₁₋₄ alkoxy, C₁₋₄ alkylthio, C₁₋₄ alkylamino, C₁₋₄ alkylsulfonyl, C₁₋₄ alkylsulfonylamino, C₁₋₄ alkylcarbonyl, C₁₋₄ alkylcarbonylamino and C₁₋₄ alkylcarboxy, each optionally substituted with one or more substituents independently selected from the group consisting of halo, hydroxy, C₁₋₄ alkoxy, amino, C₁₋₄ alkylamino, di-(C₁₋₄ alkyl)amino and cyano, (b) halo, (e) hydroxy, (f) amino and (g) oxo groups, and Z² being (i) a further 3- to 6-membered carbocyclic or heterocyclic ring, optionally substituted with no more than two Z¹ groups as defined above, or (ii) NR⁷R⁸ where R⁷ and R⁸ are as defined above;
or a pharmaceutically acceptable salt thereof.

In a specific embodiment, the selective Bcl-2 inhibitor provided herein is 2-(1H-pyrrolo[2,3-b]pyridin-5-yloxy)-4-(4-((2-(4-chlorophenyl)-4,4-dimethylcyclohex-1-enyl)methyl)piperazin-1-yl)-N-(3-nitro-4-(((tetrahydro-2H-pyran-4-yl)methyl)amino)phenylsulfonyl)benzamide (also known as ABT-199 or venetoclax), which is described in U.S. Pat. No. 8,546,399 and WO2012/121758, and has the following formula or pharmaceutically acceptable salt thereof:

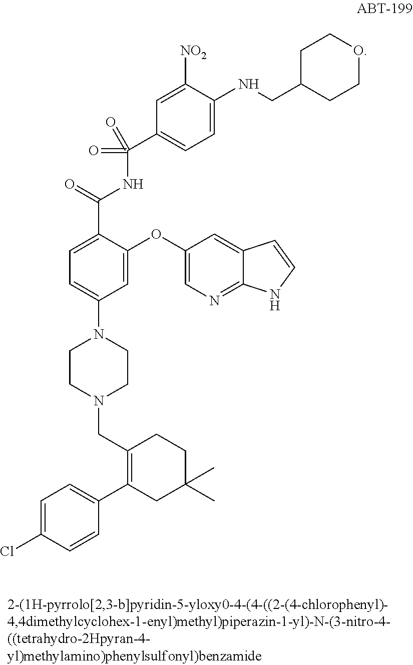

In a specific embodiment, provided herein is a combination therapy comprising APR-246 and ABT-199 (venetoclax). In a specific embodiment, provided herein is a combination therapy comprising Compound A and ABT-199 (venetoclax).

In another specific embodiment, provided herein is a combination therapy comprising Compound B and ABT-199 (venetoclax). In yet another specific embodiment, provided herein is a combination therapy comprising Compound C and ABT-199 (venetoclax). In yet another specific embodiment, provided herein is a combination therapy comprising Compound D and ABT-199 (venetoclax). In yet another specific embodiment, provided herein is a combination therapy comprising Compound E and ABT-199 (venetoclax).
6.2.3 Mcl-1 Inhibitors

As described above, myeloid cell leukemia-1 (Mcl-1) is an antiapoptotic Bcl-2 family protein. Mcl-1 is a key regulator of mitochondrial homeostasis. Frequent overexpression of MCL-1 in human primary and drug-resistant cancer cells makes it an attractive cancer therapeutic target. As shown in Section 7 below, the p53 reactivators provided herein, when used in combination with Mcl-1 inhibitors, generate synergistic effects. Thus, in one aspect, provided herein is a combination therapy comprising a p53 reactivator and a Mcl-1 inhibitor.

In some embodiments, the Mcl-1 inhibitor provided herein is selected from the group consisting of AT101 (R-(−)-gossypol), TW-37, Gambogic acid, Sabutoclax (BI-97C1), Marinopyrrole A (maritoclax), UMI-77, A-1210477, MIK665, AMG-176, AZD5991, Flavopiridol, Roscovitine, CR8, Voruciclib (P 1446A-05), Cardiac glycosides UNBS1450, Benzyl isothiocyanate, BAY43-9006, BEZ235 AZD8055, and Arsenic trioxide Bufalin.

In a specific embodiment, the Mcl-1 inhibitor is AMG-176 having the following formula:

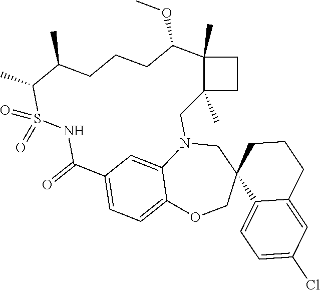 [SMILES: CO[C@H]1CCC[C@H](C)[C@@H](C)S(=O)(=O)NC(=O)c2ccc3c(c2)N(C[C@@]2(CCCc4cc(Cl)ccc42)CO3)C[C@]2(C)CC[C@@]12C]

(Spiro[5,7-etheno-1H,11H-cyclobut[i][1,4]oxazepino[3,4-f][1,2,7]thiadiazacyclohexadecine-2(3H),1′(2′H)-naphthalen]-8(9H)-one, 6′-chloro-3′,4′,12,13,16,16a,17,18,18a,19-decahydro-16-methoxy-11,12-dimethyl-,10,10-dioxide, (1′S,11R,12S,14E,16S,16aR,18aR)-).

In another specific embodiment, the Mcl-1 inhibitor is MIK665 having the following formula:

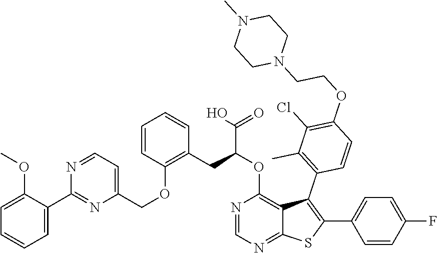

((R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoic acid)

In a specific embodiment, provided herein is a combination therapy comprising APR-246 and AMG-176. In a specific embodiment, provided herein is a combination therapy comprising APR-246 and MIK665.
6.2.4 Antibodies Binding CD20

In some embodiments, the antibody binding CD20 (anti-CD20 antibody) provided herein is a monoclonal antibody (mAb).

In some embodiments, the anti-CD20 antibody provided herein is rituximab (Biogen and Genentech), obinutuzumab (Roche), ocaratuzumab (Mentrik Biotech), ofatumumab (Genmab), ocrelizumab (Genentech), ibritumomab (Biogen), or veltuzumab (Immunomedics).

In a specific embodiment, the anti-CD20 antibody is rituximab (RITUXAN®).

6.3 Pharmaceutical Compositions

The p53 reactivator provided herein can be formulated in a pharmaceutical composition that comprises a p53 reactivator provided herein and a pharmaceutically acceptable excipient. Similarly, an inhibitor of an antiapoptotic Bcl-2 family protein (e.g., a Bcl-2 inhibitor or a Mcl-1 inhibitor) provided herein can be formulated in a pharmaceutical composition that comprises an inhibitor of an antiapoptotic Bcl-2 family protein (e.g., a Bcl-2 inhibitor or a Mcl-1 inhibitor) provided herein and a pharmaceutically acceptable excipient. In some embodiments, provided herein is a combination therapy comprising a first pharmaceutical composition comprising a p53 reactivator provided herein and a first pharmaceutically acceptable excipient, and a second pharmaceutical composition comprising a Bcl-2 inhibitor or a Mcl-1 inhibitor provided herein and a second pharmaceutically acceptable excipient. In some embodiments, a p53 reactivator provided herein and a Bcl-2 inhibitor provided herein are formulated together in a pharmaceutical composition. In some embodiments, a p53 reactivator provided herein and a Mcl-1 inhibitor provided herein are formulated together in a pharmaceutical composition. In other embodiments, provided herein is a pharmaceutical composition comprising a p53 reactivator provided herein, a Bcl-2 inhibitor provided herein, and a pharmaceutically acceptable excipient. In other embodiments, provided herein is a pharmaceutical composition comprising a p53 reactivator provided herein, a Mcl-1 inhibitor provided herein, and a pharmaceutically acceptable excipient.

Similarly, the anti-CD20 antibody provided herein (e.g., rituximab) can be formulated in a pharmaceutical composition that comprises the anti-CD20 antibody provided herein (e.g., rituximab) and a pharmaceutically acceptable excipient. In some embodiments, provided herein is a combination therapy comprising a first pharmaceutical composition comprising a p53 reactivator provided herein and a first pharmaceutically acceptable excipient, a second pharmaceutical composition comprising a Bcl-2 inhibitor provided herein and a second pharmaceutically acceptable excipient, and a third pharmaceutical composition comprising an anti-CD20 antibody provided herein (e.g., rituximab) and a third pharmaceutically acceptable excipient. The first, the second, and the third pharmaceutically acceptable excipients can be the same or different. In some embodiments, a p53 reactivator provided herein and a Bcl-2 inhibitor provided herein are formulated in a single pharmaceutical composition. In other embodiments, provided herein is a pharmaceutical composition comprising a p53 reactivator provided herein, a Bcl-2 inhibitor provided herein, and a pharmaceutically acceptable excipient.

The p53 reactivator and/or Bcl-2 inhibitor or Mcl-1 inhibitor provided herein can be formulated into suitable pharmaceutical compositions for different routes of administration, such as injection, sublingual and buccal, rectal, vaginal, ocular, otic, nasal, inhalation, nebulization, cutaneous, or transdermal. The compounds described above may be formulated into pharmaceutical compositions using techniques and procedures well known in the art (see, e.g., Ansel, Introduction to Pharmaceutical Dosage Forms, (7th ed. 1999)).

The p53 reactivator, Bcl-2 inhibitor, and/or the anti-CD20 antibody (e.g., rituximab) provided herein can be formulated into suitable pharmaceutical compositions for different routes of administration, such as injection, sublingual and buccal, rectal, vaginal, ocular, otic, nasal, inhalation, nebulization, cutaneous, or transdermal. The compounds described above may be formulated into pharmaceutical compositions using techniques and procedures well known in the art (see, e.g., Ansel, Introduction to Pharmaceutical Dosage Forms, (7th ed. 1999)).

In the compositions, effective concentrations of one or more compounds (i.e., p53 reactivators or Bcl-2 inhibitors or Mcl-1 inhibitors provided herein) or pharmaceutically acceptable salts are mixed with a suitable pharmaceutical excipient. In certain embodiments, the concentrations of the compounds in the compositions are effective for delivery of an amount, upon administration, that treats, prevents, or ameliorates one or more of the symptoms and/or progression of a disease or disorder provided herein (e.g., cancer, including solid cancer and blood borne cancer).

The active compound is in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans. The concentration of active compound in the pharmaceutical composition will depend on absorption, tissue distribution, inactivation, and excretion rates of the active compound, the physicochemical characteristics of the compound, the dosage schedule, and amount administered as well as other factors known to those of skill in the art.

The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical excipients. Examples of unit dose forms include ampoules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules, or bottles of pints or gallons. Hence, multiple dose form is a multiple of unit doses which are not segregated in packaging.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Solutions or suspensions used for parenteral, intradermal, subcutaneous, or topical application can include any of the following components: a sterile diluents (such as water, saline solution, fixed oil, polyethylene glycol, glycerine, propylene glycol, dimethyl acetamide, or other synthetic solvent), antimicrobial agents (such as benzyl alcohol and methyl parabens), antioxidants (such as ascorbic acid and sodium bisulfate), chelating agents (such as ethylenediaminetetraacetic acid (EDTA)), buffers (such as acetates, citrates, and phosphates), and agents for the adjustment of tonicity (such as sodium chloride or dextrose). Parenteral preparations can be enclosed in ampoules, pens, disposable syringes, or single or multiple dose vials made of glass, plastic, or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolving the compound in aqueous sodium hydroxide, sodium bicarbonate, or hydrochloric acid.

Sustained-release preparations can also be prepared. Suitable examples of sustained-release preparations include semipermeable matrices of solid hydrophobic polymers containing the p53 reactivator or Bcl-2 inhibitor or Mcl-1 inhibitor provided herein, which matrices are in the form of shaped articles, e.g., films or microcapsule. Examples of sustained-release matrices include iontophoresis patches, polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Lactose-free compositions provided herein can contain excipients that are well known in the art. In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch, and magnesium stearate.

Further encompassed are anhydrous pharmaceutical compositions and dosage forms containing a p53 reactivator or Bcl-2 inhibitor or Mcl-1 inhibitor provided herein. Anhydrous pharmaceutical compositions and dosage forms provided herein can be prepared using anhydrous or low moisture containing ingredients and low moisture or low humidity conditions, as known by those skilled in the art. An anhydrous pharmaceutical composition should be prepared and stored such that its anhydrous nature is maintained. Accordingly, anhydrous compositions are packaged using materials known to prevent exposure to water such that they can be included in suitable formulatory kits. Examples of suitable packaging include, but are not limited to, hermetically sealed foils, plastics, unit dose containers (e.g., vials), blister packs, and strip packs.

Dosage forms or compositions containing active ingredient in the range of 0.001% to 100% with the balance made up from non-toxic carrier may be prepared. In some embodiments, the contemplated compositions contain from about 0.005% to about 95% active ingredient. In other embodiments, the contemplated compositions contain from about 0.01% to about 90% active ingredient. In certain embodiments, the contemplated compositions contain from about 0.1% to about 85% active ingredient. In other embodiments, the contemplated compositions contain from about 0.1% to about 75-95% active ingredient.

Parenteral administration of the compositions includes intravenous, subcutaneous, and intramuscular administrations. Compositions for parenteral administration include sterile solutions ready for injection, sterile dry soluble products, such as lyophilized powders, ready to be combined with a solvent just prior to use, sterile suspensions ready for injection, and sterile emulsions. The solutions may be either aqueous or nonaqueous. The unit dose parenteral preparations can be packaged in an ampoule, a vial or a syringe with a needle.

Pharmaceutically acceptable excipients used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents, and other pharmaceutically acceptable substances.

Examples of aqueous excipients include sodium chloride injection, Ringer's injection, isotonic dextrose injection, sterile water injection, dextrose and lactated Ringer's injection. Nonaqueous parenteral vehicles include fixed oils of vegetable origin, such as cottonseed oil, corn oil, sesame oil, and peanut oil. Antimicrobial agents in bacteriostatic or fungistatic concentrations must be added to parenteral preparations packaged in multiple dose containers, which include phenols or cresols, mercurials, benzyl alcohol, chlorobutanol, methyl and propyl-p-hydroxybenzoic acid esters, thimerosal, benzalkonium chloride, and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical excipients also include ethyl alcohol, polyethylene glycol and propylene glycol for water miscible vehicles, and sodium hydroxide, hydrochloric acid, citric acid, or lactic acid for pH adjustment.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

Lyophilized powders are of interest here, which can be reconstituted for administration as solutions, emulsions, and other mixtures. They may also be reconstituted and formulated as solids or gels.

The sterile, lyophilized powder is prepared by dissolving a p53 reactivator or Bcl-2 inhibitor or Mcl-1 inhibitor provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose, or other suitable agent. The solvent may also contain a buffer, such as citrate, phosphate, or other buffers known to those of skill in the art. Subsequent sterile filtration of the solution followed by lyophilization under standard conditions known to those of skill in the art provides the desired formulation. Generally, the resulting solution will be apportioned into vials for lyophilization. Each vial will contain a single dosage or multiple dosages of the p53 reactivator or Bcl-2 inhibitor or Mcl-1 inhibitor. The lyophilized powder can be stored under appropriate conditions, such as at about 4° C. to room temperature.

In one aspect, the lyophilized formulations are suitable for reconstitution with a suitable diluent to the appropriate concentration prior to administration. In one embodiment, the lyophilized formulation is stable at room temperature. In one embodiment, the lyophilized formulation is stable at room temperature for up to about 24 months. In one embodiment, the lyophilized formulation is stable at room temperature for up to about 24 months, up to about 18 months, up to about 12 months, up to about 6 months, up to about 3 months or up to about 1 month. In one embodiment, the lyophilized formulation is stable upon storage under accelerated condition of 40° C./75% RH for up to about 12 months, up to about 6 months or up to about 3 months.

In some embodiments, the lyophilized formulation is suitable for reconstitution with an aqueous solution for intravenous administrations. In certain embodiments, the lyophilized formulation provided herein is suitable for reconstitution with water. In one embodiment, the reconstituted aqueous solution is stable at room temperature for up to about 24 hours upon reconsititution. In one embodiment, the reconstituted aqueous solution is stable at room temperature from about 1-24, 2-20, 2-15, 2-10 hours upon reconsititution. In one embodiment, the reconstituted aqueous solution is stable at room temperature for up to about 20, 15, 12, 10, 8, 6, 4 or 2 hours upon reconsititution. In certain embodiments, the lyophilized formulations upon reconstitution have a pH of about 4 to 5.

In certain embodiment, the lyophilized formulations comprise a p53 reactivator or Bcl-2 inhibitor or Mcl-1 inhibitor provided herein, a buffer and a bulking agent.

In one embodiment, the lyophilized formulation comprises about 0.1-2% comprise a p53 reactivator or Bcl-2 inhibitor or Mcl-1 inhibitor provided herein, about 1-15% buffer and about 70-95% bulking agent based on the total weight of the lyophilized formulation.

In certain embodiments, a lyophilized formulation comprises a p53 reactivator or Bcl-2 inhibitor or Mcl-1 inhibitor provided herein, in about 0.1 to about 2% based on the total weight of the lyophilized formulation. In some embodiments, a lyophilized formulation comprises a p53 reactivator and/or Bcl-2 inhibitor or Mcl-1 inhibitor provided herein, in an amount of about 0.1 mg to about 5 mg in a vial, for example, a 20 ml vial.

In certain embodiments, a lyophilized formulation comprises a citrate buffer in an amount from about 5% to about 25% based on total weight of the lyophilized formulation. In one embodiment, the citrate buffer comprises anhydrous citric acid and anhydrous sodium citrate.

In some embodiments, the bulking agent in the lyophilized formulations comprises Captisol®, mannitol or Kleptose®, for example, β-cyclodextrin, hydroxypropyl β-cyclodextrin and methylated β-cyclodextrin.

The lyophilized formulation can be reconstituted for parenteral administration to a patient using any pharmaceutically acceptable diluent. Such diluents include, but are not limited to Sterile Water for Injection (SWFI), Dextrose 5% in Water (D5W), or a cosolvent system. Any quantity of diluent may be used to reconstitute the lyophilized formulation such that a suitable solution for injection is prepared. Accordingly, the quantity of the diluent must be sufficient to dissolve the lyophilized formulation. In one embodiment, 1-5 mL or 1-3 mL of a diluent are used to reconstitute the lyophilized formulation to yield a final concentration of about 0.1-5 mg/mL, about 0.1-1 mg/mL, or about 0.5-1 mg/mL of a p53 reactivator and/or Bcl-2 inhibitor or Mcl-1 inhibitor provided herein. In certain embodiments, the final concentration of a p53 reactivator or Bcl-2 inhibitor or Mcl-1 inhibitor provided herein, in the reconstituted solution is about 0.5 mg/mL. In certain embodiment, the volume of the reconstitution diluent varies between 2 ml and 20 ml to yield a final concentration of 0.05-0.5 mg/mL. In certain embodiment, depending on the required dose, multiple vials may be used for reconstitution.

Topical mixtures are prepared as described for the local and systemic administration. The resulting mixture may be a solution, suspension, emulsion, or the like and are formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

The p53 reactivator or Bcl-2 inhibitors or Mcl-1 inhibitors or pharmaceutically acceptable salts thereof may be formulated as aerosols for topical application, such as by inhalation. These formulations for administration to the respiratory tract can be in the form of an aerosol or solution for a nebulizer, or as a microfine powder for insufflation, alone or in combination with an inert excipients such as lactose. In such a case, the particles of the formulation will have diameters of less than 50 microns or less than 10 microns.

These solutions, particularly those intended for ophthalmic use, may be formulated as 0.01% -10% isotonic solutions, pH about 5-7, with appropriate salts.

Other routes of administration such as transdermal patches and rectal administration are also contemplated herein.

For example, pharmaceutical dosage forms for rectal administration are rectal suppositories, capsules, and tablets for systemic effect. Rectal suppositories as used herein mean solid bodies for insertion into the rectum, which melt or soften at body temperature releasing one or more pharmacologically or therapeutically active ingredients. Pharmaceutically acceptable substances utilized in rectal suppositories include bases (or vehicles) and agents that raise the melting point. Examples of bases include, for example, cocoa butter (theobroma oil), glycerin gelatin, carbowax (polyoxyethylene glycol), and appropriate mixtures of mono, di and triglycerides of fatty acids. Combinations of the various bases may be used. Agents to raise the melting point of suppositories include, for example, spermaceti and wax. Rectal suppositories may be prepared either by the compressed method or by molding. An exemplary weight of a rectal suppository is about 2 to 3 grams.

The p53 reactivator and/or Bcl-2 inhibitor or Mcl-1 inhibitor provided herein can be administered by controlled release means or by delivery devices that are well known to those of ordinary skill in the art. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof, to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side effects (e.g., adverse effects).

Most controlled-release formulations are designed to initially release an amount of drug (active ingredient) that promptly produces the desired therapeutic effect, then to gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, other physiological conditions, or compounds.

In certain embodiments, the agent may be administered using intravenous infusion, an implantable osmotic pump, a transdermal patch, liposomes, or other modes of administration. In one embodiment, a pump may be used. In another embodiment, polymeric materials can be used. In yet another embodiment, a controlled release system can be placed in proximity of the therapeutic target, thus requiring only a fraction of the systemic dose. See, e.g., Goodson, Medical Applications of Controlled Release, vol. 2, pp. 115-138 (1984).

In some embodiments, a controlled release device is introduced into a subject in proximity of the site of inappropriate immune activation or a tumor. The active ingredient can be dispersed in a solid inner matrix (e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate). In some embodiments, the inner matrix is surrounded by an outer polymeric membrane (e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene, propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer). In certain embodiments, the outer polymeric membrane is insoluble in body fluids. The active ingredient then diffuses through the outer polymeric membrane in a release rate controlling step. The percentage of active ingredient contained in such parenteral compositions depends on the specific nature thereof, as well as the needs of the subject.

The p53 reactivators or Bcl-2 inhibitors or Mcl-1 inhibitors provided herein, or pharmaceutically acceptable salts thereof, may also be formulated to target a particular tissue, receptor, or other area of the body of the subject to be treated. Many such targeting methods are well known to those of skill in the art. All such targeting methods are contemplated herein for use in the instant compositions. In one embodiment, liposomal suspensions, including tissue-targeted liposomes, such as tumor-targeted liposomes, may also be suitable as pharmaceutically acceptable excipients. These may be prepared according to methods known to those skilled in the art.

The p53 reactivators or Bcl-2 inhibitors or anti-CD20 antibodies (e.g., rituximab) provided herein, or pharmaceutically acceptable salts thereof, may also be formulated to target a particular tissue, receptor, or other area of the body of the subject to be treated. Many such targeting methods are well known to those of skill in the art. All such targeting methods are contemplated herein for use in the instant compositions. In one embodiment, liposomal suspensions, including tissue-targeted liposomes, such as tumor-targeted liposomes, may also be suitable as pharmaceutically acceptable excipients. These may be prepared according to methods known to those skilled in the art.
6.3.1 A Liquid Composition Comprising A P53 Reactivator

In some specific embodiments, the agonist of p53 such as a p53 reactivator provided here (e.g., APR-246 or Compound A) is formulated in an aqueous solution, as described in U.S. Pat. No. 9,061,016, which is incorporated herein by reference, and as described in more detail below.

In some specific embodiments, the p53 reactivator provided here (e.g., APR-246 or Compound A) is formulated in an aqueous solution, as described in U.S. Pat. No. 9,061,016, which is incorporated herein by reference, and as described in more detail below.

In one embodiment, the formulation comprising the p53 reactivator is a stock solution and preferably is a pharmaceutical formulation in the form of a concentrated stock solution. The formulation preferably is sterile, and this may be achieved by known sterilization methods such as filtration, allowing for long term storage essentially without any deterioration of the p53 reactivator, e.g. by a chemical reaction of degradation, and essentially without formation of degradation products.

The formulation provided herein can be used, e.g. for administration to a patient in need thereof by direct injection or preferentially diluted with appropriate injectable solutions for i.v. infusion.

In one embodiment, the formulation provided herein is an aqueous solution of the p53 reactivator provided herein (e.g., APR-246 or Compound A), wherein the p53 reactivator is present at a concentration within a range of about 10 mg/mL to about 250 mg/mL, a range of about 50 mg/mL to about 200 mg/mL, or a range of about 75 mg/mL to about 150 mg/mL of the formulation.

The formulation may be diluted prior to use, e.g., administration to a patient. The dilution factor depends on the concentration of the p53 reactivator in the formulation and the required amount of the compound needed, e.g., to meet the therapeutically effective dose. In some embodiments, in case of parenteral administration, the final diluted product has a pH within the range of about pH 4 to about pH 6. In some embodiments, the final diluted product for parenteral administration has a pH within the range of about pH 4.2 to about pH 5.5.

The liquid formulation may contain sodium chloride at a concentration of between 0% and 3%, a concentration of between 0.5% and 1.5%, or a concentration of between 0.8% and 1% weight by volume of the formulation.

In one embodiment, the p53 reactivator (e.g., APR-246 or Compound A) is present in the liquid formulation in the form of an acid addition salt with one or several different pharmaceutically acceptable acids. The pharmaceutically acceptable acid may be a mineral acid, e.g., selected from the group consisting of hydrochloric acid, hydrogen bromide, hydrogen iodide, sulphuric acid, nitric acid, phosphoric acid and the like. As an alternative, the pharmaceutically acceptable acid may be an organic acid, e.g., a sulfonic or carboxylic acid, particularly an alkyl or aryl sulfonic acid or an alkyl or aryl carboxylic acid, such as selected from the group consisting of methanesulfonic acid, p-toluenesulfonic acid, benzenesulfonic acid, acetic acid, tartaric acid, maleic acid, citric acid, benzoic acid, salicylic acid, ascorbic acid and the like.

In some embodiments, to be at the required pH, the composition provided herein contains a pH regulating agent. The term “pH regulating agent,” as used herein, means at least one pharmaceutically acceptable organic or inorganic (mineral) acid, or at least one pharmaceutically acceptable acid buffer or a mixture of any of these. Thus, the pH regulating agent may be any such acid or buffer, or a mixture of acids or buffers, or a mixture of acid(s) and buffer(s). Examples of useful acids and buffers are as indicated herein.

For example, the composition may contain at least one pharmaceutically acceptable acid. The acid may be an inorganic mineral acid, e.g., selected from the group consisting of hydrochloric acid, hydrobromic acid, hydroiodic acid, sulphuric acid, nitric acid, phosphoric acid or the like, or an organic acid, e.g., selected from the group consisting of acetic acid, succinic acid, tartaric acid, maleic acid, ascorbic acid, citric acid, glutamic acid, benzoic acid, ascorbic acid, methanesulfonic acid, ethanesulfonic acid and the like. It is contemplated that the composition may contain one or several acids, selected from inorganic and organic acids. In one embodiment, the required pH of the formulation is achieved by addition of hydrochloric acid.

The composition provided herein also may comprise at least one pharmaceutically acceptable buffer, particularly selected from the group of citric buffer, acetate buffer, phosphate buffer and the like, separately or as a mixture thereof, as well as in combination with any pharmaceutically acceptable acid, as defined herein, e.g., hydrochloric acid.

The liquid composition provided herein is aqueous, which means that it contains water. However, it is contemplated that the aqueous solution and the aqueous phase used to prepare the composition also may contain other pharmaceutically acceptable liquids as a solvent phase, e.g., polyethylene glycol (PEG) and alcohols, e.g., ethanol. In some embodiments, the agueous phase mainly comprises water as a solvent. For example, the solvent phase is comprised of from 50 to 100% water, at least 80% water, at least 90% water, at least 95% water, at least 98% water or 100% water.

In one embodiment, the composition described herein is provided as a stable stock solution, particularly as a concentrated stock solution for long term storage at a temperature range of 2-8° C., in a container, for example, a sealed and sterilized container. For example, the composition may comprise a stable aqueous WFI (water for injection) solution of the p53 reactivator, optionally as an acid addition salt, in particular a hydrochloride addition salt, in a concentration of at about 10 mg/mL to about 250 mg/mL, at about 50 mg/mL to about 200 mg/mL, or at about 75 mg/mL to about 150 mg/mL, and a pH regulating agent in such an amount as to provide a pH in the solution in a range of between pH 3.0 and pH 5.0, between pH 3.2 and pH 4.7, between pH 3.5 and pH 4.5, or between pH 3.8 and pH 4.2, e.g., approximately 4.0. For example, the pH of the stock solution may have a lower limit selected from a pH of about 3.0, or about 3.2, e.g. about 3.4, such as about 3.6 or about 3.8, and an upper limit of about 5.0, or about 4.7, or about 4.5, or about 4.2, e.g. about 4.0.

Other components also may be added to or present in the aqueous phase, such as pharmaceutically acceptable inorganic salts, e.g., NaCl, preservatives, or further pharmaceutically acceptable compounds, e.g., further therapeutically active ingredients, such as cytostatics, particularly cisplatin, daunorubicin, cerubidine, cytarabine and fludarabine.

In one embodiment, NaCl is added to the aqueous phase in an amount so as to provide a final liquid composition as defined herein above, containing NaCl at a concentration of between 0% and 3%, between 0.5% and 1.5%, or between 0.8% and 1% weight by volume of the formulation.

In one embodiment, the composition is a sterile formulation. In this case, sterilization of the composition may be accomplished by passing the formulation, e.g., a formulated stock solution, through a sterile filter with a nominal pore size of 0.2 μm into a cleaned and sterilized container.

The composition may be provided as a ready-to-use injection solution, wherein a liquid formulation, e.g., a stock solution, is brought to the desired volume by addition of one or more pharmaceutically acceptable solvents, such as selected from the group consisting of WFI, a glucose solution, electrolyte solution containing amino acids, lipids, vitamins, and other minerals, Ringer's solution, Hartmann's solution, or a sodium chloride solution in the form of an isotonic, hypotonic or hypertonic solution. An example of such pharmaceutically acceptable solution is Baxter Viaflo 9 mg/ml.

In a specific embodiment, the p53 reactivator is APR-246, which is formulated in liquid formulation, which comprises at least one pH regulating agent in an amount such as to provide a pH in the aqueous solution of from about 3.0 to about 5.0. In some embodiments, APR-246 is present in the aqueous solution at a concentration of from 10 mg/mL to 250 mg/mL. In some embodiments, the aqueous solution comprises NaCl at a concentration of between 0% to 3% weight by volume. In another specific embodiment, the p53 reactivator is Compound A.

In another specific embodiment, the p53 reactivator is Compound B. In yet another specific embodiment, the p53 reactivator is Compound C. In yet another specific embodiment, the p53 reactivator is Compound D. In yet another specific embodiment, the p53 reactivator is Compound E.
6.3.2 An Oral Dosage Form Comprising a P53 Reactivator

In some specific embodiments, the agonist of p53 such as the p53 reactivator provided here (e.g., Compound A) is formulated in a composition for oral administration. In a specific embodiment, the oral dosage form is a solid form.

In some specific embodiments, the p53 reactivator provided here (e.g., Compound A) is formulated in a composition for oral administration. In a specific embodiment, the oral dosage form is a solid form.

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Typical oral dosage forms are prepared by combining the active ingredients in an intimate admixture with at least one excipient according to conventional pharmaceutical compounding techniques. Excipients can take a wide variety of forms depending on the form of preparation desired for administration. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105, and mixtures thereof. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants are used in compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil, zinc stearate, ethyl oleate, ethyl laureate, agar, and mixtures thereof. Additional lubricants include, for example, a syloid silica gel, a coagulated aerosol of synthetic silica, and mixtures thereof.
6.3.3 A Solid Dispersion Comprising a Bcl-2 Inhibitor

In some more specific embodiments, the Bcl-2 inhibitor provided herein (e.g., ABT-199) is present in the solid dispersion in its parent-compound form, alone or together with a salt form of the compound. In some embodiments, the Bcl-2 inhibitor provided herein (e.g., ABT-199) is present in the solid dispersion in its parent-compound form. In some embodiments, the Bcl-2 inhibitor provided herein (e.g., ABT-199) forms acid addition salts, basic addition salts or zwitterions.

Acid addition salts are those derived from reaction of the Bcl-2 inhibitor provided herein (e.g., ABT-199) with an acid. For example, salts including the acetate, adipate, alginate, ascorbate, bicarbonate, citrate, aspartate, benzoate, benzenesulfonate (besylate), bisulfate, butyrate, camphorate, camphorsulfonate, digluconate, ethanedisulfonate, formate, fumarate, glycerophosphate, glutamate, hemisulfate, heptanoate, hexanoate, hydrobromide, hydrochloride, hydroiodide, 1-hydroxy-2-naphthoate, lactate, lactobionate, malate, maleate, malonate, mesitylenesulfonate, methanesulfonate, naphthalenesulfonate, nicotinate, oxalate, pamoate, pectinate, persulfate, phosphate, picrate, propionate, succinate, sulfate, tartrate, thiocyanate, trichloroacetate, trifluoroacetate, para-toluenesulfonate and undecanoate salts of the Bcl-2 inhibitor provided herein (e.g., ABT-199) can be used in the present disclosure.

Basic addition salts, including those derived from reaction of a compound with the bicarbonate, carbonate, hydroxide or phosphate of cations such as lithium, sodium, potassium, calcium and magnesium, can be used.

In some embodiments, the Bcl-2 inhibitor provided herein (e.g., ABT-199) has more than one protonatable nitrogen atom and thus is capable of forming acid addition salts with more than one, for example about 1.2 to about 2, about 1.5 to about 2 or about 1.8 to about 2, equivalents of acid per equivalent of the compound.

In some embodiments, the Bcl-2 inhibitor provided herein (e.g., ABT-199) or a salt thereof is present in a solid dispersion as described in WO2012/121758. In some embodiments, the Bcl-2 inhibitor provided herein is dispersed in a solid matrix that comprises (a) at least one pharmaceutically acceptable water-soluble polymeric carrier and (b) at least one pharmaceutically acceptable surfactant. More detailed description of the pharmaceutically acceptable water-soluble polymeric carrier and pharmaceutically acceptable surfactant is provided in WO2012/121758 and in the following paragraphs.

More specifically, in some embodiments, the major component of the matrix of a solid dispersion product is a polymer that is hydrophilic or water-soluble at least in a part of the pH scale, more particularly at a pH occurring in the gastrointestinal (GI) tract, or a combination of such polymers.

One or more polymeric carriers typically constitute in total about 20% to about 95%, such as about 20% to about 90%, for example about 40% to about 85%, or about 60% to about 85%, or about 70% to about 85%, or even about 75% to about 85%, by weight of the solid dispersion.

In some embodiments, a polymer or polymer mixture useful herein is solid at ambient temperature and should remain solid even at the highest temperatures typically experienced during storage, transport and handling of the product. Suitable water-soluble polymers include, but are not limited to, those having a glass transition temperature (T_{g}) of at least about 40° C., at least about 50° C., at least about 60° C., or more, or about 80° C. to about 180° C.

Water-soluble polymers are polymers that form a clear homogeneous solution in water. Typically, a homogeneous solution is essentially uniform throughout, and appears clear under visual inspection or alternatively using an instrument such as a turbidimeter. For example, when dissolved at 20° C. in an aqueous solution at 2% (w/v), a water-soluble polymer may have an apparent viscosity of about 1 to about 5000 mPa·s, for example about 1 to about 700 mPa·s, or about 5 to about 100 mPa·s.

Non-limiting examples of polymeric carriers useful herein include those described in WO2012/121758, for example, homopolymers and copolymers of N-vinyl lactams, especially homopolymers and copolymers of N-vinyl pyrrolidone, e.g., the homopolymer polyvinylpyrrolidone (PVP or povidone) and copolymers such as those comprising monomers of N-vinyl pyrrolidone and vinyl acetate (copovidone) or N-vinyl pyrrolidone and vinyl propionate; cellulose esters and cellulose ethers, in particular methylcellulose, ethylcellulose, (hydroxyalkyl)celluloses such as hydroxypropylcellulose, (hydroxyalkyl)alkyl-celluloses such as hydroxypropylmethylcellulose (HPMC or hypromellose), cellulose phthalates and succinates such as cellulose acetate phthalate, hydroxypropylmethylcellulose phthalate (HPMC-P), hydroxypropylmethylcellulose succinate (HPMC-S) and hydroxypropylmethylcellulose acetate succinate (HPMC-AS); high molecular weight polyalkylene oxides such as polyethylene oxides (PEGs or PEOs) and copolymers of ethylene oxide and propylene oxide (poloxamers); polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates) and poly(hydroxyalkyl methacrylates); polyacrylamides; vinyl acetate polymers such as copolymers of vinyl acetate and crotonic acid, polyvinyl acetate, polyvinyl alcohol and partially hydrolyzed polyvinyl acetate (also referred to as partially saponified polyvinyl alcohol); graft copolymers of polyethylene glycol, polyvinyl caprolactam and polyvinyl acetate (e.g., Soluplus™ of BASF or equivalent product); oligo- and polysaccharides such as carrageenans, galactomannans and xanthan gum; and mixtures of two or more thereof.

In a specific embodiment, the copovidone provided herein is one consisting of about 60% N-vinyl pyrrolidone and about 40% vinyl acetate monomers (copovidone 60/40).

In some embodiments, HPMCs and derivatives thereof provided herein include, but not limited to, HPMC E3, HPMC E5, HPMC E6, HPMC E15, HPMC K3, HPMC A4, HPMC A15, HPMC-AS LF, HPMC-AS MF, HPMC-AS HF, HPMC-AS LG, HPMC-AS MG, HPMC-AS HG, HPMC-P 50, HPMC-P 55 and combinations thereof.

In one embodiment, the solid dispersion matrix comprises one or more polymeric carriers selected from the group consisting of povidones, copovidones, HPMCs, polyethylene glycol/polyvinyl caprolactam/polyvinyl acetate graft copolymers and mixtures thereof. In a more particular embodiment, the solid dispersion matrix comprises one or more polymeric carriers selected from the group consisting of povidone K30, copovidone 60/40, HPMC E5, Soluplus™ polyethylene glycol/polyvinyl caprolactam/polyvinyl acetate graft copolymer and products equivalent to Soluplus™.

The surfactant component can be anionic, non-ionic or can comprise a combination of anionic and non-ionic surfactants. One or more surfactants typically constitute in total about 2% to about 25%, for example about 5% to about 20%, such as between about 5% and about 15%, or between about 5% and about 10% by weight of the solid dispersion.

In some embodiments, the surfactants provided herein herein are pharmaceutically acceptable non-ionic surfactants. Non-limiting examples of non-ionic surfactants provided herein include those described in WO2012/121758, for example, polyoxyethylene castor oil derivatives such as PEG-35 castor oil (e.g., Cremophor EL™ of BASF or equivalent product), PEG-40 hydrogenated castor oil (e.g., Cremophor RH™ 40 or equivalent product) and PEG-60 hydrogenated castor oil (e.g., Cremophor RH™ 60 or equivalent product); other polyoxyethylene glycerides such as PEG-32 glyceryl laurate (e.g., Gelucire™ 44/14 of Gattefosse or equivalent product) and PEG-32 glyceryl palmitostearate (e.g., Gelucire™ 50/13 or equivalent product), and Labrafil Ml 944 CS (oleoyl macrogol 6 glycerides prepared by transesterification of apricot kernel oil with PEG 300); fatty acid monoesters of sorbitan, for example sorbitan monooleate (e.g., Span™ 80 or equivalent product), sorbitan monostearate (e.g., Span™ 60 or equivalent product), sorbitan monopalmitate (e.g., Span™ 40 or equivalent product) and sorbitan monolaurate (e.g. , Span™ 20 or equivalent product); other fatty acid esters of sorbitan, for example, sorbitan tristearate and sorbitan trioleate; fatty acid monoesters of polyoxyethylene sorbitan (polysorbates) such as PEG-20 sorbitan monooleate (polysorbate 80, e.g., Tween™ 80 or equivalent product) PEG-20 sorbitan monostearate (polysorbate 60, e.g., Tween™ 60 or equivalent product), PEG-20 sorbitan monopalmitate (polysorbate 40, e.g., Tween™ 40 or equivalent product), or PEG-20 sorbitan monolaurate (polysorbate 20, e.g., Tween™ 20 or equivalent product); other fatty acid esters of polyoxyethylene sorbitan, for example, polyoxyethylene (20) sorbitan tristearate (Tween 65), polyoxyethylene (20) sorbitan trioleate (Tween 85); fatty acid ester of polyalkylene glycols such as, for example, PEG 660 hydroxy-stearic acid (polyglycol ester of 12-hydroxystearic acid (70 mol %) with 30 mol % ethylene glycol); polyalkoxylated ethers of fatty alcohols such as, for example, PEG (2) stearyl ether (Brij 72), macrogol 6 cetylstearyl ether or macrogol 25 cetylstearyl ether; a tocopheryl compound such as a-tocopheryl polyethylene glycol succinate, which is commonly abbreviated as vitamin E-TPGS (Vitamin E-TPGS is a water-soluble form of natural-source vitamin E prepared by esterifying d-alpha-tocopheryl acid succinate with polyethylene glycol 1000); and mixtures of two or more thereof.

In one embodiment, the solid dispersion comprises one or more surfactants selected from the group consisting of polyoxyethylene glycerides (including polyoxyethylene castor oil derivatives), polysorbates, TPGS and mixtures thereof. In a more particular embodiment, the solid dispersion matrix comprises one or more polymeric carriers selected from the group consisting of PEG-40 hydrogenated castor oil, polysorbate 80, polysorbate 20 and TPGS.

In some embodiments, the solid dispersion provided herein comprises other components, such as one or more lubricants, glidants or flow regulators. In some embodiments, the solid dispersion provided herein comprises colloidal silicon dioxide or fumed silica (e.g., Aerosil). In some embodiments, the solid dispersion contains one or more bulking agents (fillers), disintegrants, cosolvents such as propylene glycol esters of fatty acids (e.g., propylene glycol laurate), plasticizers and/or stabilizers such as antioxidants, light stabilizers, free radical scavengers or antimicrobial agents.

Exemplary lubricants include glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and combinations thereof.

Exemplary anti-adherents include talc, colloidal silicon dioxide, starch, DL-leucine, sodium lauryl sulfate and metallic stearates.

Exemplary glidants include colloidal silicon dioxide, starch, powdered cellulose, sodium lauryl sulfate, magnesium trisilicate and metallic stearates.

Exemplary diluents include lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydro lyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of a- and amorphous cellulose and powdered cellulose, and cellulose acetate; calcium salts including calcium carbonate, tribasic calcium phosphate, dicalcium phosphate (e.g., dibasic calcium phosphate dihydrate), monobasic calcium sulfate monohydrate, calcium sulfate and granular calcium lactate trihydrate; magnesium carbonate; magnesium oxide; bentonite; kaolin; sodium chloride; and combinations thereof.

Exemplary disintegrants include starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and combinations thereof.

Exemplary wetting agents include quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters, for example sorbitan monolaurate, sorbitan monooleate, sorbitan monopalmitate and sorbitan monostearate; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; a-tocopherol polyethylene glycol (1000) succinate (TPGS); tyloxapol; and combinations thereof.

Exemplary binding agents and adhesives include acacia; tragacanth; glucose; polydextrose; starch including pregelatinized starch; gelatin; modified celluloses including methylcellulose, carmellose sodium, hydroxypropylmethylcellulose (HPMC), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone or PVP), for example povidone K-15, K-30 and K-29/32; polyacrylic acids (carbomers); polymethacrylates; and combinations thereof.

Other excipients such as buffering agents, stabilizers, antioxidants, antimicrobials, colorants, flavors and sweeteners are known in the pharmaceutical art and can be used in compositions of the present invention.

In some specific embodiments, in the solid dispersion, the Bcl-2 inhibitor or salt thereof is present in a parent-compound-equivalent amount of about 5% to about 40% by weight. In some embodiments, in the solid dispersion, the at least one polymeric carrier is present in an amount of about 40% to about 85% by weight and the at least one surfactant is present in an amount of about 5% to about 20% by weight. In some embodiments, in the solid dispersion, the at least one polymeric carrier is selected from the group consisting of homopolymers and copolymers of N-vinyl lactams, cellulose esters, cellulose ethers, high molecular weight polyalkylene oxides, polyacrylates, polymethacrylates, polyacrylamides, vinyl acetate polymers, graft copolymers of polyethylene glycol, polyvinyl caprolactam and polyvinyl acetate, oligo- and polysaccharides and mixtures thereof. In some embodiments, the at least one polymeric carrier is selected from the group consisting of povidones, copovidones, HPMCs, polyethylene glycol/polyvinyl caprolactam/polyvinyl acetate graft copolymers and mixtures thereof. In some embodiments, the at least one surfactant is non-ionic. In some embodiments, the at least one surfactant is selected from the group consisting of polyoxyethylene glycerides, fatty acid monoesters of sorbitan, polysorbates, a-tocopheryl polyethylene glycol succinate (TPGS) and mixtures thereof. In some embodiments, the solid dispersion further comprises at least one glidant. In some embodiments, the at least one glidant comprises colloidal silicon dioxide.

In some specific embodiments, in the solid dispersion, the Bcl-2 inhibitor or salt thereof is present in a parent-compound-equivalent amount of about 5% to about 40% by weight, the at least one polymeric carrier is present in an amount of about 40% to about 85% by weight and the at least one surfactant is present in an amount of about 5% to about 20% by weight. In other embodiments, in the solid dispersion, the Bcl-2 inhibitor or salt thereof is present in a parent-compound-equivalent amount of about 5% to about 15% by weight, the at least one polymeric carrier is present in an amount of about 70% to about 85% by weight and the at least one surfactant is present in an amount of about 5% to about 15% by weight

In a specific embodiment, the Bcl-2 inhibitor is ABT-199, and the solid dispersion comprises a polymeric carrier—a copovidone, and a surfactant—a polysorbate. In some embodiments, the solid dispersion further comprises at least one glidant, which optionally comprises colloidal silicon dioxide.

6.4 Uses for Treatment of A Disease or Disorder and Dosing
6.4.1 Combination Therapies of a p53 reactivator and a Bcl-2 inhibitor or Mcl-1 inhibitor

Provided herein are combination treatments with an agonist of p53 such as a p53 reactivator (see Section 6.2.1) with a Bcl-2 inhibitor (see Section 6.2.2) or with a Mcl-1 Inhibitor (see Section 6.2.3) see Section).

In some embodiments, provided herein is a composition or a combination of compositions for use in the prevention and/or treatment of a disease or condition comprising the p53 reactivator and an inhibitor of an antiapoptotic Bcl-2 family protein (e.g., a Bcl-2 inhibitor or a Mcl-1 inhibitor) provided herein. In one embodiment, provided herein is a composition or a combination of compositions for use in the prevention of a disease or condition, comprising the p53 reactivator and an inhibitor of an antiapoptotic Bcl-2 family protein (e.g., a Bcl-2 inhibitor or a Mcl-1 inhibitor) provided herein. In one embodiment, provided herein is a composition or a combination of compositions for use in the treatment of a disease or condition, comprising the p53 reactivator and an inhibitor of an antiapoptotic Bcl-2 family protein (e.g., a Bcl-2 inhibitor or a Mcl-1 inhibitor) provided herein.

In other embodiments, provided herein is a method of preventing and/or treating a disease or condition in a subject, comprising administering an effective amount of the p53 reactivator and the Bcl-2 inhibitor or Mcl-1 inhibitor provided herein or pharmaceutical compositions thereof. In some embodiments, provided herein is a method of preventing a disease or condition in a subject, comprising administering an effective amount of the p53 reactivator and the Bcl-2 inhibitor or Mcl-1 inhibitor provided herein or pharmaceutical compositions thereof. In other embodiments, provided herein is a method of treating a disease or condition in a subject, comprising administering an effective amount of the p53 reactivator and the Bcl-2 inhibitor or Mcl-1 inhibitor provided herein or pharmaceutical compositions thereof.

The subject administered a therapy can be a mammal such as non-primate (e.g., cows, pigs, horses, cats, dogs, rats etc.) or a primate (e.g., a monkey, such as a cynomolgus monkey, or a human). In a one embodiment, the subject is a human. In another embodiment, the subject is a human with a disease or condition.

In some embodiments, the disease or disorder is a mutant p53 mediated cancer (including, e.g., hematological tumors with mutations in the p53 gene). In other embodiments, the disease or disorder is not a mutant p53 mediated cancer.

In some embodiments, the disease or disorder is a disease characterized by apoptotic dysfunction and/or overexpression of an anti-apoptotic Bcl-2 family protein (e.g., Bcl-2).

In some embodiments, the disease or disorder is mutant p53 mediated and characterized by apoptotic dysfunction and/or overexpression of an anti-apoptotic Bcl-2 family protein (e.g., Bcl-2).

In some embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In some embodiments, the disease or disorder is a hematological cancer, such as leukemia, lymphoma, or myeloma. In some embodiments, the cancer is selected from the group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments, the solid tumor cancer is selected from the group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

In other embodiments, the disease or disorder is an immune or autoimmune disorder. Such disorders include autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis-associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemo lytic anaemia, autoimmune hepatitis, type-1 autoimmune hepatitis (classical autoimmune or lupoid hepatitis), autoimmune mediated hypoglycemia, autoimmune neutropenia, autoimmune thrombocytopenia, autoimmune thyroid disease, B-cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy-associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinemia), conjunctivitis, connective tissue disease-associated interstitial lung disease, contact dermatitis, Coombs-positive hemolytic anemia, cor pulmonale, Creutzfeldt-Jakob disease, cryptogenic autoimmune hepatitis, cryptogenic fibrosing alveolitis, culture-negative sepsis, cystic fibrosis, cytokine therapy-associated disorders, Crohn's disease, dementia pugilistica, demyelinating diseases, dengue hemorrhagic fever, dermatitis, dermatitis scleroderma, dermatologic conditions, dermatomyositis/polymyositis-associated lung disease, diabetes, diabetic arteriosclerotic disease, diabetes mellitus, diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram-negative sepsis, gram-positive sepsis, granulomas due to intracellular organisms, group B streptococci (GBS) infection, Graves' disease, hemosiderosis-associated lung disease, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpura, hepatitis A, hepatitis B, hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopenia, idiopathic pulmonary fibrosis, idiopathic thrombocytopenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease-associated lung disease, monoclonal gammopathy, multiple myeloma, multiple systems degenerations (Mencel, Dejerine-Thomas, Shy-Drager and Machado-Joseph), myalgic encephalitis/Royal Free Disease, myasthenia gravis, microscopic vasculitis of the kidneys, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, non-alcoholic steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis, Pneumocystis carinii pneumonia, pneumonia, POEMS syndrome (polyneuropathy, organomegaly, endocrinopathy, monoclonal gammopathy, and skin changes syndrome), post-perfusion syndrome, post-pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, progressive supranuclear palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis-associated interstitial lung disease, rheumatoid spondylitis, sarcoidosis, Schmidt's syndrome, scleroderma, senile chorea, senile dementia of Lewy body type, sepsis syndrome, septic shock, seronegative arthropathies, shock, sickle cell anemia, T-cell or FAB ALL, Takayasu's disease/arteritis, telangiectasia, Th2-type and Thl-type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, yersinia and salmonella-associated arthropathy, acquired immunodeficiency disease syndrome (AIDS), autoimmune lymphoproliferative syndrome, hemolytic anemia, inflammatory diseases, thrombocytopenia, acute and chronic immune diseases associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), Sjogren's disease-associated lung disease, Sjogren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, sporadic polyglandular deficiency type I, sporadic polyglandular deficiency type II, Still's disease, streptococcal myositis, stroke, structural lesions of the cerebellum, subacute sclerosing panencephalitis, sympathetic ophthalmia, syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, lupus nephritis, systemic sclerosis, and systemic sclerosis-associated interstitial lung disease.

Various delivery systems are known and can be used to administer a prophylactic or therapeutic agent (e.g., the p53 reactivator, Bcl-2 inhibitor or Mcl-1 inhibitor provided herein), including, but not limited to, parenteral administration (e.g., intradermal, intramuscular, intraperitoneal, intravenous and subcutaneous), epidural, and mucosal (e.g., intranasal and oral routes).

In a specific embodiment, a prophylactic or therapeutic agent (e.g., the p53 reactivator and/or Bcl-2 inhibitor or Mcl-1 inhibitor provided herein), or a pharmaceutical composition is administered intranasally, intramuscularly, intravenously, or subcutaneously. In another specific embodiment, a prophylactic or therapeutic agent (e.g., the p53 reactivator and/or Bcl-2 inhibitor or Mcl-1 inhibitor provided herein), or a pharmaceutical composition is administered orally. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In another embodiment, a prophylactic or therapeutic agent, or a composition provided herein can be delivered in a vesicle, in particular a liposome. In another embodiment, a prophylactic or therapeutic agent, or a composition provided herein can be delivered in a controlled release or sustained release system as described in the above section.

The amount of a prophylactic or therapeutic agent (the p53 reactivator and the Bcl-2 inhibitor or Mcl-1 inhibitor provided herein), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and in some embodiments, should be decided according to the judgment of the practitioner and each patient's circumstances. Effective doses may also be extrapolated from dose-response curves derived from in vitro or animal model test systems.

The dose administered to a mammal, particularly a human, in the context of the present disclosure should be sufficient to effect a therapeutic response in the mammal over a reasonable time frame. One skilled in the art will recognize that dosage will depend upon a variety of factors including the potency of the specific compound, the age, condition and body weight of the patient, as well as the stage/severity of the disease. The dose will also be determined by the route (administration form) timing and frequency of administration.

The p53 reactivator (e.g., APR-246) and the Bcl-2 inhibitor (e.g., ABT-199) can be formulated in different pharmaceutical compositions and administered separately to the subject in need thereof. Alternatively, the p53 reactivator (e.g., APR-246) and the Bcl-2 inhibitor (e.g., ABT-199) are administered together in the same pharmaceutical composition. Similarly, the p53 reactivator (e.g., APR-246) and the Mcl-1 inhibitor (e.g., AMG-176 or MIK665) can be formulated in different pharmaceutical compositions and administered separately to the subject in need thereof. Alternatively, the p53 reactivator (e.g., APR-246) and the Mcl-1 inhibitor (e.g., AMG-176 or MIK665) are administered together in the same pharmaceutical composition.

In some embodiments, the p53 reactivator (e.g., APR-246) and the Bcl-2 inhibitor (e.g., ABT-199) are administered simultaneously. In some embodiments, the p53 reactivator (e.g., APR-246) and the Mcl-1 inhibitor (e.g., AMG-176 or MIK665) are administered simultaneously. The term “simultaneously” means at the same time or within a short period of time, for example, less than 1 hour, less than 2 hours, less than 3 hours, less than 4 hours, or less than 12 hours.

In some embodiments, the p53 reactivator (e.g., APR-246) and the Bcl-2 inhibitor (e.g., ABT-199) or the Mcl-1 inhibitor (e.g., AMG-176 or MIK665) are not administered simultaneously, and instead the two compounds are administered at different times. In some embodiments, the p53 reactivator (e.g., APR-246) and the Bcl-2 inhibitor (e.g., ABT-199) or the Mcl-1 inhibitor (e.g., AMG-176 or MIK665) are administered at least once during a dosing period. A dosing period as used herein is meant a period of time, during which each therapeutic agent has been administered at least once. A dosing cycle can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, a dosing cycle is 1, 2, 3, 4, 5, 6, 7, 8, 9, or 10 weeks. In certain embodiments, a dosing period is a dosing cycle.

The prophylactic or therapeutic agent (the p53 reactivator and/or the Bcl-2 inhibitor or the Mcl-1 inhibitor provided herein) can be delivered as a single dose (e.g., a single bolus injection), or over time (e.g., continuous infusion over time or divided bolus doses over time). The agent can be administered repeatedly if necessary, for example, until the patient experiences stable disease or regression, or until the patient experiences disease progression or unacceptable toxicity. Stable disease or lack is determined by methods known in the art such as evaluation of patient symptoms, physical examination, and visualization of the tumor that has been imaged using X-ray, CAT, PET, MRI scan, or other commonly accepted evaluation modalities.

The prophylactic or therapeutic agent (the p53 reactivator and/or the Bcl-2 inhibitor or the Mcl-1 inhibitor provided herein) can be administered once daily (QD) or divided into multiple daily doses such as twice daily (BID), three times daily (TID), and four times daily (QID). In addition, the administration can be continuous (i.e., daily for consecutive days or every day) or intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of, e.g., at least 10 days. The term “intermittent” or “intermittently” as used herein is intended to mean stopping and starting at either regular or irregular intervals. For example, intermittent administration of the compound is administration for one to six days per week, administration in cycles (e.g., daily administration for two to eight consecutive weeks, then a rest period with no administration for up to one week), or administration on alternate days. The term “cycling” as used herein is intended to mean that a therapeutic compound is administered daily or continuously but with a rest period. In certain embodiments, the rest period is the same length as the treatment period. In other embodiments, the rest period has different length from the treatment period. In some embodiments, the length of cycling is 1, 2, 3, 4, 5, 6, 7, 8, 9, or 10 weeks. In some embodiments of cycling, a therapeutic compound is administered daily for a period of 1, 2, 3, 4, 5, 6, 7, 8, 9, 10, 15, 20, 25, or 30 days, followed by a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks.

In certain embodiments, the compound is administered once per day from one day to six months, from one week to three months, from one week to four weeks, from one week to three weeks, or from one week to two weeks.
6.4.2 Combination Therapies of a p53 Reactivator, a Bcl-2 Inhibitor, and an Anti-CD20 Antibody

Also provided herein are combination therapies using a p53 reactivator (such as the p53 reactivator described in Section 6.2.1) in combination with a Bcl-2 inhibitor (such as the Bcl-2 inhibitor described in Section 6.2.2) and an anti-CD20 antibody (such as rituximab) for treating a disease or disorder (such as lymphoma, e.g., a non-Hodgkin lymphoma).

Provided herein are methods of treating a disease or disorder (e.g., lymphoma) in a subject comprising administering to a subject a p53 reactivator (a compound that can give reactivation of a mutant p53), a Bcl-2 inhibitor, and an anti-CD20 monoclonal antibody (mAb).

In certain embodiments, provided herein are methods of treating a disease or disorder (e.g., lymphoma) in a subject comprising administering to a subject a p53 reactivator (a compound that can give reactivation of a mutant p53), a Bcl-2 inhibitor, and rituximab. In certain embodiments, a pharmaceutically effective amount of the p53 reactivator is administered. In certain embodiments, a pharmaceutically effective amount of the Bcl-2 inhibitor is administered. In certain embodiments, a pharmaceutically effective amount of rituximab is administered. In certain embodiments, the p53 reactivator, the Bcl-2 inhibitor, and rituximab are concomitantly administered. In certain embodiments, the co-administeration of the p53 reactivator, the Bcl-2 inhibitor, and rituximab is pharmaceutically effective to treat the disease or disorder (e.g., non-Hodgkin lymphoma).

In some embodiments, the subject is a human. In some embodiments, the subject is a subject diagnosed with lymphoma. In some embodiments, the subject is a subject diagnosed with non-Hodgkin lymphoma (NHL). In some embodiments, the subject is a subject diagnosed with mature (peripheral) B-cell neoplasm. In some preferred embodiments, the subject is a subject diagnosed with chronic lymphocytic leukemia (CLL) or mantle cell lymphoma (MCL). In one embodiment, the subject is a subject diagnosed with chronic lymphocytic leukemia (CLL). In another embodiment, the subject is a subject diagnosed with cell lymphoma (MCL). In yet another embodiment, the subject is a subject diagnosed with relapsed and/or refractory (R/R) CLL or relapsed and/or refractory (R/R) MCL. In yet another embodiment, the subject is a subject diagnosed with R/R CLL. In yet another embodiment, the subject is subject diagnosed with R/R MCL.

In some embodiments, the subject is diagnosed to have TP53 mutation. In some embodiments, the subject carries TP53 mutation. In some embodiments, the subject has mutant p53 protein. In some embodiments, the subject is a subject diagnosed with a TP53 mutant lymphoma. In some embodiments, the subject is a subject diagnosed with a TP53 mutant non-Hodgkin lymphoma (NHL). In some embodiments, the subject is a subject diagnosed with a TP53 mutant mature (peripheral) B-cell neoplasm. In some preferred embodiments, the subject is a subject diagnosed with a TP53 mutant chronic lymphocytic leukemia (CLL) or mantle cell lymphoma (MCL). In one embodiment, the subject is a subject diagnosed with a TP53 mutant chronic lymphocytic leukemia (CLL). In another embodiment, the subject is a subject diagnosed with a TP53 mutant mantle cell lymphoma (MCL). In yet another embodiment, the subject is a subject diagnosed with a TP53 mutant R/R CLL or a TP53 mutant R/R MCL. In yet another embodiment, the subject is a subject diagnosed with a TP53 mutant R/R CLL. In yet another embodiment, the subject is a subject diagnosed with a TP53 mutant R/R MCL.

In some embodiments, the methods described herein further comprises determining by gene sequencing if the subject has TP53 mutation.

In some embodiments, the disease or disorder is a mutant p53 mediated cancer (including, e.g., hematological tumors with mutations in the p53 gene). In other embodiments, the disease or disorder is not a mutant p53 mediated cancer.

In some embodiments, the disease or disorder is a disease characterized by apoptotic dysfunction and/or overexpression of a Bcl-2 protein and/or CD20.

In some embodiments, the disease or disorder is mutant p53 mediated and characterized by apoptotic dysfunction and/or overexpression of Bcl-2 protein and/or CD20.

In some embodiments, the disease or disorder is a hematological cancer, such as leukemia, lymphoma, or myeloma. In some embodiments, the disease or disorder is a lymphoma. In some embodiments, the disease or disorder is Hodgkin's lymphoma or non-Hodgkin's lymphoma (NHL). In some embodiments, the disease or disorder is a Hodgkin lymphoma. In some embodiments, the disease or disorder is a non-Hodgkin lymphoma (NHL). In some embodiments, the disease or disorder is a mature (peripheral) B-cell neoplasm. In some embodiments, the disease or disorder a non-Hodgkin lymphoma (NHL) selected from the group consisting of: chronic lymphocytic leukemia (CLL), mantle cell lymphoma (MCL), B-cell prolymphocytic leukemia, diffuse large cell B-cell lymphoma (DLBCL), lymphoplasmacytic lymphoma, splenic marginal zone B-cell lymphoma, hairy cell leukemia, plasma cell myeloma (plasmacytoma), extranodal marginal zone B-cell lymphoma, nodal marginal zone lymphoma, follicle center lymphoma, and Burkitt's leukemia (Burkitt's lymphoma). In some preferred embodiments, the disease or disorder is chronic lymphocytic leukemia (CLL) or mantle cell lymphoma (MCL). In some preferred embodiments, the disease or disorder is chronic lymphocytic leukemia (CLL). In some preferred embodiments, the disease or disorder is mantle cell lymphoma (MCL).

In some embodiments, the disease or disorder is a TP53 mutant lymphoma. In some embodiments, the disease or disorder is a TP53 mutant non-Hodgkin lymphoma (NHL). In some embodiments, the disease or disorder is a TP53 mutant mature (peripheral) B-cell neoplasm. In some preferred embodiments, the disease or disorder is a TP53 mutant chronic lymphocytic leukemia (CLL) or mantle cell lymphoma (MCL). In one embodiment, the disease or disorder is a TP53 mutant chronic lymphocytic leukemia (CLL). In another embodiment, the disease or disorder is a TP53 mutant mantle cell lymphoma (MCL). In yet another embodiment, the disease or disorder is a TP53 mutant R/R CLL or a TP53 mutant R/R MCL. In yet another embodiment, the disease or disorder is a TP53 mutant R/R CLL. In yet another embodiment, the disease or disorder is a TP53 mutant R/R MCL.

In some embodiments, the mutation in TP53 is R248Q, R248W, R273H, R273C, R175H, Y220C, G245S, R249S, R282W, V173A, S241F, R249S or a combination thereof. In some embodiments, the mutant p53 contains at least one replacement in the core domain of p53 (residues 94-292) caused by a TP53 mutation. In other embodiments, the mutant TP53 includes a nonsense mutation. A nonsense mutation is a genetic mutation changing a codon for an amino acid into a stop codon, resulting in a shorter, unfinished protein product. Nonsense mutations are less frequent than missense mutations in TP53, but nonetheless constitute about 10% of all TP53 mutations in cancer. The most common TP53 nonsense mutation yields a truncated p53; R213X aka R213*.

In some embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, and retinoblastoma.

In some embodiments, the disease or disorder is selected from the group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia.

In some embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In other embodiments, the disease or disorder is a solid tumor cancer. In some embodiments, the solid tumor cancer is selected from the group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

The amounts of the p53 reactivator, the Bcl-2 inhibitor, and rituximab provided herein, or a pharmaceutical composition that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and in some embodiments, should be decided according to the judgment of the practitioner and each patient's circumstances. Effective doses may also be extrapolated from dose-response curves derived from in vitro or animal model test systems.

In some embodiments, the p53 reactivator is administered to the subject as part a composition. In some embodiments, the composition is a pharmaceutical composition described in Section 6.3.

In some embodiments, the p53 reactivator (e.g., APR-246), the Bcl-2 inhibitor (e.g., venetoclax), and rituximab are administered simultaneously. The term “simultaneously” means at the same time or within a short period of time, for example, less than 1 hour, less than 2 hours, less than 3 hours, less than 4 hours, or less than 12 hours.

In some embodiments, the p53 reactivator (e.g., APR-246), the Bcl-2 inhibitor (e.g., venetoclax), and rituximab are not administered simultaneously, and instead the three compounds are administered at different times.

In certain embodiments, the subject has not been previously treated with any Bcl-2 inhibitor (e.g., venetoclax) prior to the co-administration of the p53 reactivator (e.g., APR-246), the Bcl-2 inhibitor (e.g., venetoclax), and rituximab.

In certain embodiments, the co-administration of the p53 reactivator (e.g., APR-246), the Bcl-2 inhibitor (e.g., venetoclax), and rituximab is concomitant administration.

In certain embodiments, the co-administration of the p53 reactivator (e.g., APR-246), the Bcl-2 inhibitor (e.g., venetoclax), and rituximab is pharmaceutically effective to treat lymphoma.

In some embodiments, the p53 reactivator (e.g., APR-246), the Bcl-2 inhibitor (e.g., venetoclax), and rituximab are administered at least once during a dosing period. A dosing period as used herein is meant a period of time, during which each therapeutic agent has been administered at least once. A dosing cycle can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, a dosing cycle is 1, 2, 3, 4, 5, 6, 7, 8, 9, or 10 weeks. In certain embodiments, a dosing period is a dosing cycle.

The prophylactic or therapeutic agent (the p53 reactivator, the Bcl-2 inhibitor provided herein, and/or rituximab) can be delivered as a single dose (e.g., a single bolus injection), or over time (e.g., continuous infusion over time or divided bolus doses over time). The agent can be administered repeatedly if necessary, for example, until the patient experiences stable disease or regression, or until the patient experiences disease progression or unacceptable toxicity. Stable disease or lack is determined by methods known in the art such as evaluation of patient symptoms, physical examination, and visualization of the tumor that has been imaged using X-ray, CAT, PET, Mill scan, or other commonly accepted evaluation modalities.

The prophylactic or therapeutic agent (the p53 reactivator, the Bcl-2 inhibitor provided herein, and/or rituximab) can be administered once daily (QD) or divided into multiple daily doses such as twice daily (BID), three times daily (TID), and four times daily (QID). In addition, the administration can be continuous (i.e., daily for consecutive days or every day) or intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of, e.g., at least 10 days. The term “intermittent” or “intermittently” as used herein is intended to mean stopping and starting at either regular or irregular intervals. For example, intermittent administration of the compound is administration for one to six days per week, administration in cycles (e.g., daily administration for two to eight consecutive weeks, then a rest period with no administration for up to one week), or administration on alternate days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks.

In certain embodiments, the compound is administered once per day from one day to six months, from one week to three months, from one week to four weeks, from one week to three weeks, or from one week to two weeks.

In some embodiments, APR-246 is administered at a dose of less than about 150 mg/kg. In some embodiments, APR-246 is administered at a dose of less than about 100 mg/kg. In some embodiments, APR-246 is administered at a dose of between about 100 mg/kg and about 25 mg/kg. In other embodiments, APR-246 is administered at dose of less than about 75 mg/kg. In yet other embodiments, the APR-246 is administered at a dose of less than about 65 mg/kg. In yet other embodiments, the APR-246 is administered at a dose of less than about 50 mg/kg.

In other embodiments, APR-246 is administered at a fixed dose within the interval of 2.7-7.5 g. In some embodiments, the fixed dose of APR-246 is no more than about 4.5 g. In some embodiments, the fixed dose of APR-246 is no more than about 4.0 g. In some embodiments, the fixed dose of APR-246 is no more than about 3.5 g. In one embodiments, APR-246 is administered at a dose of about 4.5 g/day, about 4.0 g/day, or about 3.5 g/day. In one specific embodiment, APR-246 is administered at a dose of about 4.5 g/day. In another specific embodiment, APR-246 is administered at a dose of about 4.0 g/day. In yet another specific embodiment, APR-246 is administered at a dose of about 3.5 g/day.

In some embodiments, APR-246 is administered in a multiple-step administration to avoid high plasma concentration and/or to minimize the risk of adverse events. In some embodiments, APR-246 is administered in a 2-step administration consisting of a first loading dose and a subsequent maintenance dose. In some preferred embodiments, the first loading dose is not equal to the subsequent maintenance dose. In other embodiments, the first loading dose is not equal to the subsequent maintenance dose. In some embodiments, the first loading dose is about 1.5 g, about 1.33 g, or about 1.16 g. In some embodiments, the subsequent maintenance dose is about 3.0 g, about 2.67 g, or about 2.34 g.

In some embodiment, APR-246 is administered 1, 2, 3, 4, 5, 6, 7, or 8 times of each cycle of 14 days, 21 days, every 28 days, every 35 days, or every 42 days. In preferred embodiments, APR-246 is administered for 4 days in each treatment cycle. In preferred embodiments, APR-246 is administered for 4 days in each treatment cycle of 28 days. In some embodiments, APR-246 is administered for at least 1, 2, 3, 4, 5, 6, 7, 8, 9, or 10 treatment cycles.

In a specific embodiment, APR-246 is administered once daily for 4 consecutive days in each treatment cycle of 28 days.

In some preferred embodiments, APR-246 is administered intravenously.

In some embodiments, the Bcl-2 inhibitor described herein (e.g., venetoclax) is administered at a dose between 1 mg/day to 1000 mg/day. In some embodiments, the Bcl-2 inhibitor is administered at a dose of about 100 mg/day, about 200 mg/day, about 300 mg/day, about 400 mg/day, about 400 mg/day, about 500 mg/day, about 600 mg/day, about 700 mg/day, about 800 mg/day, about 900 mg/day, or about 1000 mg/day. In some embodiments, Bcl-2 inhibitor is administered at a dose of about 400 mg/day.

In some preferred embodiments, venetoclax is administered at a dose of about 400 mg. In one preferred embodiment, the Bcl-2 inhibitor (e.g., venetoclax) is administered daily. In one embodiment, venetoclax is administered daily for each treatment cycle of 28 days.

In some embodiments, venetoclax is administered to the subject in a ramp-up phase prior to the co-administration. In some embodiments, the ramp-up phase is a period of 5 weeks. In some embodiments, venetoclax is administered daily at a dose lower than 400 mg/day during the ramp-up phase. In some embodiments, venetoclax is administered daily at a dose of about 20 mg/day, about 50 mg/day, about 100 mg/day, or about 200 mg/day during the ramp-up phase. In a specific embodiment, venetoclax is administered daily at a dose of about 20 mg/day during week 1, at a dose of about 50 mg/day during week 2, at a dose of about 100 mg/day during week 3, at a dose of about 200 mg/day during week 4, at a dose of about 400 mg/day during week 5 of the ramp-up phase.

In some embodiments, the Bcl-2 inhibitor (e.g., venetoclax) is administered for at least 1, 2, 3, 4, 5, 6, 7, 8, 9, or 10 treatment cycles.

In some preferred embodiments, the Bcl-2 inhibitor (e.g., venetoclax) is administered orally.

In some embodiments, rituximab is administered at a dose between 1 mg/m² to 1000 mg/m². In some embodiments, rituximab is administered at a dose of about 100 mg/m², about 200 mg/m², about 300 mg/m², about 400 mg/m², about 500 mg/m², about 600 mg/m², about 700 mg/m², about 800 mg/m², about 900 mg/m², or about 1000 mg/m². In some embodiments, rituximab is administered at a dose of about 375 mg/m² or about 500 mg/m². In one embodiment, rituximab is administered at a dose of about 375 mg/m². In another embodiment, rituximab is administered at a dose of about 500 mg/m².

In some embodiment, rituximab is administered 1, 2, 3, 4, 5, 6, 7, or 8 times of each cycle of 14 days, 21 days, every 28 days, every 35 days, or every 42 days. In preferred embodiments, rituximab is administered once in each treatment cycle. In preferred embodiments, rituximab is administered once in each treatment cycle of 28 days. In some embodiments, rituximab is administered once for at least 1, 2, 3, 4, 5, 6, 7, 8, 9, or 10 treatment cycles. In some preferred embodiments, rituximab is administered intravenously.

In certain embodiments, combination treatment of the p53 reactivator (e.g., APR-246), the Bcl-2 inhibitor (e.g., venetoclax), and rituximab shows synergistic effects in treating lymphoma.

In some embodiments, APR-246 is administered at a dose of about 4.5 g/day for 4 days in combination with venetoclax administered daily at a dose of about 400 mg and rituximab administered once at a dose of about 375 mg/m² or about 500 mg/m² in each 28-day cycle. In some specific embodiments, APR-246 is administered at a dose about 4.5 g/day on Days 1-4 in combination with venetoclax administered daily at a dose of about 400 mg and rituximab administered once at a dose of about 375 mg/m² or about 500 mg/m² on Day 5 in each 28-day cycle. In some embodiments, APR-246, venetoclax, and rituximab are administered for 1 to 10 cycles. In some embodiments, APR-246, venetoclax, and rituximab are administered for 1 cycle. In some embodiments, APR-246, venetoclax, and rituximab are administered for 2 cycles. In some embodiments, APR-246, venetoclax, and rituximab are administered for 3 cycles. In some embodiments, APR-246, venetoclax, and rituximab are administered for 4 cycles. In some embodiments, APR-246, venetoclax, and rituximab are administered for 5 cycles. In some embodiments, APR-246, venetoclax, and rituximab are administered for 6 cycles. In some embodiments, APR-246, venetoclax, and rituximab are administered for 7 cycles. In some embodiments, APR-246, venetoclax, and rituximab are administered for 8 cycles. In some embodiments, APR-246, venetoclax, and rituximab are administered for 9 cycles. In some embodiments, APR-246, venetoclax, and rituximab are administered for 10 cycles. In some embodiments, APR-246, venetoclax, and rituximab are administered for more than 10 cycles.

In some embodiments, APR-246 is administered at a dose of about 4.0 g/day for 4 days in combination with venetoclax administered daily at a dose of about 400 mg and rituximab administered once at a dose of about 375 mg/m² or about 500 mg/m² in each 28-day cycle. In some specific embodiments, APR-246 is administered at a dose about 4.5 g/day on Days 1-4 in combination with venetoclax administered daily at a dose of about 400 mg and rituximab administered once at a dose of about 375 mg/m² or about 500 mg/m² on Day 5 in each 28-day cycle. In some embodiments, APR-246, venetoclax, and rituximab are administered for 1 to 10 cycles. In some embodiments, APR-246, venetoclax, and rituximab are administered for 1 cycle. In some embodiments, APR-246, venetoclax, and rituximab are administered for 2 cycles. In some embodiments, APR-246, venetoclax, and rituximab are administered for 3 cycles. In some embodiments, APR-246, venetoclax, and rituximab are administered for 4 cycles. In some embodiments, APR-246, venetoclax, and rituximab are administered for 5 cycles. In some embodiments, APR-246, venetoclax, and rituximab are administered for 6 cycles. In some embodiments, APR-246, venetoclax, and rituximab are administered for 7 cycles. In some embodiments, APR-246, venetoclax, and rituximab are administered for 8 cycles. In some embodiments, APR-246, venetoclax, and rituximab are administered for 9 cycles. In some embodiments, APR-246, venetoclax, and rituximab are administered for 10 cycles. In some embodiments, APR-246, venetoclax, and rituximab are administered for more than 10 cycles.

In some embodiments, APR-246 is administered at a dose of about 3.5 g/day for 4 days in combination with venetoclax administered daily at a dose of about 400 mg and rituximab administered once at a dose of about 375 mg/m² or about 500 mg/m² in each 28-day cycle. In some specific embodiments, APR-246 is administered at a dose about 4.5 g/day on Days 1-4 in combination with venetoclax administered daily at a dose of about 400 mg and rituximab administered once at a dose of about 375 mg/m² or about 500 mg/m² on Day 5 in each 28-day cycle. In some embodiments, APR-246, venetoclax, and rituximab are administered for 1 to 10 cycles. In some embodiments, APR-246, venetoclax, and rituximab are administered for 1 cycle. In some embodiments, APR-246, venetoclax, and rituximab are administered for 2 cycles. In some embodiments, APR-246, venetoclax, and rituximab are administered for 3 cycles. In some embodiments, APR-246, venetoclax, and rituximab are administered for 4 cycles. In some embodiments, APR-246, venetoclax, and rituximab are administered for 5 cycles. In some embodiments, APR-246, venetoclax, and rituximab are administered for 6 cycles. In some embodiments, APR-246, venetoclax, and rituximab are administered for 7 cycles. In some embodiments, APR-246, venetoclax, and rituximab are administered for 8 cycles. In some embodiments, APR-246, venetoclax, and rituximab are administered for 9 cycles. In some embodiments, APR-246, venetoclax, and rituximab are administered for 10 cycles. In some embodiments, APR-246, venetoclax, and rituximab are administered for more than 10 cycles.

In certain embodiments, the methods described herein further comprises determining by gene sequencing if the subject has TP53 mutation. In certain embodiments, a next generation sequencing (NGS) method is used for the gene sequencing. In certain embodiments, the gene sequencing data are interpreted to determine if the subject has or likely has TP53 mutation.

In another aspect, also provided herein are combination therapies of the p53 reactivator, the Bcl-2 inhibitor, and the anti-CD20 mAb provided herein for use in treating a disease or disorder described herein (e.g., lymphoma). In some embodiments, also provided herein are combination therapies of the p53 reactivator, the Bcl-2 inhibitor provided herein, and rituximab for use in treating chronic lymphocytic leukemia (CLL) or mantle cell lymphoma (MCL).

In another aspect, also provided herein are pharmaceutical compositions or a combination of pharmaceutical compositions of the p53 reactivator, the Bcl-2 inhibitor, and the anti-CD20 mAb provided herein for use in treating a disease or disorder (e.g., lymphoma). In some embodiments, also provided herein are pharmaceutical compositions or a combination of pharmaceutical compositions of the p53 reactivator, the Bcl-2 inhibitor provided herein, and rituximab for use in treating chronic lymphocytic leukemia (CLL) or mantle cell lymphoma (MCL).

In another aspect, also provided herein are method of treating lymphoma in a subject, comprising administering to the subject a therapeutically effective amount of a compound that can give reactivation of a mutant p53, wherein the lymphoma does not comprise a cancer cell having mutant p53 or the lymphoma comprises a cancer cell having wild-type p53.
6.4.3 Assays for Demonstrating Synergistic Effects of a P53 Reactivator and a Bcl 2 Inhibitor

Also provided herein are assays for demonstrating the effects of the combination treatment with an agonist of p53 such as a p53 reactivator and a Bcl-2 inhibitor.

A model system for a solid tumor malignancy may be treated with a p53 reactivator or a Bcl-2 inhibitor, or a combination of the two, and the effects of the combination treatment are analyzed and compared to the monotherapies. Depending on the model system used, rituximab could also be included in the assay to demonstrate synergy.

Synergistic effects of a p53 reactivator and a Bcl-2 inhibitor can then be analyzed using well-known analytical tools.

In one embodiment, the analytical tool is COMBENEFIT. Combenefit (Di Veroli. C, Bioinformatics, 32(18), 2016, 2866-2868) is an interactive platform for the analysis and visualization of drug combinations and only requires the user to save the data in the predefined .xls template files. Combenefit performs combination analyses using the standard Loewe, Bliss and HSA methods (see Ianevski et al., Bioinformatics, 2017, 33(15): 2413-2415).

Combination effects are also analyzed using the Additive model (see Valeriote et al., Cancer Chemother Rep. 1975, 59:895-900; Lepri et al., Hematol Oncol. 1991, 9:79-86; and Jonsson E et al., Eur J Chn Pharmacol. 1998, 54:509-14.) In samples with two co-incubated substances, a predicted cell viability (%) is calculated according to the following formula: Predicted cell viability (%)=cell viability of substance 1 (%)×cell viability of substance 2 (%)×0.01.

A “combination index” (CI) is then calculated as the measured cell viability of the sample with two co-incubated substances divided by the predicted cell viability. The following classifications are used in this example:
CI>1.2 sub-additive effect
CI=0.8−1.2 additive effect
CI<0.8 synergistic effect
CI<0.5 strong synergistic effect

If the measured cell viability for a combination of two substances is higher than the cell viability for one or both substances, the effect is considered antagonistic. If the predicted viability is very low, the quote “measured viability/predicted viability” may give false CI values. Thus, a lower limit of <5% of the predicted viability may be set.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 7. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### 7.1 Example 1

### Synergistic Effects of APR-246 in Combination With a Bcl-2 Inhibitor

In this example, synergistic effects of an exemplary p53 reactivator (i.e., APR-246) in combination with an exemplary Bcl-2 inhibitor (i.e., ABT-199 or Venetoclax) were tested. A matrix combinational dose response screening of APR-246 and ABT-199 was performed in 6 AML cell lines with different p53 status.
7.1.1 Materials and Methods

Materials

For cell lines KBM3, KG-1 and HL-60: IMEM (Sigma Cat. no. I3390), 20% FBS (Sigma Cat. no. F084) and 1% L-glutamine (Sigma, Cat.no. G7513).

For MV4-11: IMEM (Sigma Cat. no. I3390), 10% FBS (Sigma Cat. no. F084) and 1% L-glutamine (Sigma, Cat.no. G7513).

For MOLM-13, RPMI (R8758, Sigma Aldrich)+10% FBS (F0804, Sigma Aldrich), heat inactivated at 56° C. for 60 min.

For SKM-1, RPMI (R8758, Sigma Aldrich)+20% FBS (F0804, Sigma Aldrich), heat inactivated at 56° C. for 60 min.

Cell Assays

Day 1

75 cm² flask (100% confluent) cells was transfer to a 15 ml tube and centrifuged at 1000 RPM for 5 min. Then supernatant was discarded, and cell pellet was resuspended in 6 ml medium. The cells were counted in the TC20 automated cell counter. 10 ml cell culture medium was prepared with 60,000 cells/ml, corresponding to 3,000 cells when 50 μl is seeded out (for MOLM-13, 12,000 cells were used). Cells were seeded in 96-well plates in column 2-12, row B-G, for blank, 100 μL cell culture medium was added. Plates were incubated overnight at 37° C.

Day 2

A given pair of drugs was combined as a series of 8 dilution (dilution factor 1.5 or 2) concentrations of ABT-199 and 7 dilution concentrations of APR-246, which resulted in a 8×7 dose matrix according to “Repl 1” Excel Combenefit template, and incubated for 72 h at 37° C.

Day 5

Plates was taken from the incubator to reach room temperature about 30 min. 100 μL CellTiter-Glo was added to all wells (except edge wells). Plates were then shaken on a plate shaker for 3 minutes at room temperature; thereafter the plates were set for 10 min at room temperature to allow cell lysis to appear. The luminescence was measured using the PerkinElmer Victor×4 instrument using the built-in program according to Instruction 11. Combenefit 2.021 was used for data analysis.

CellTiter-Glo Viability (CTG) Assay

The CellTiter-Glo® Luminescent Cell Viability Assay is a homogeneous method of determining the number of viable cells in culture based on quantitation of the ATP present, an indicator of metabolically active cells. The homogeneous assay procedure involves adding the single reagent (CellTiter-Glo® Reagent) directly to cells cultured in serum-supplemented medium. The homogeneous “add-mix-measure” format results in cell lysis and generation of a luminescent signal proportional to the amount of ATP present. The amount of ATP is directly proportional to the number of cells present in culture.

Dose Response Determinations by XLFIT

For IC50 determinations, the serial dilution of compounds was performed in cell culture medium based on 1.5- or 2-fold dilution factor. The serially diluted compounds were added to the cells and incubated for 72 h followed by cell viability test using the CTG assay. For each concentration, the current viability values were calculated and IC50 values were determined by XLfit software version 5.5.0.5 (a Microsoft Excel-based plug-in to perform regression, curve fitting and statistical analysis).

Analysis of Combinations by COMBENEFIT

Combenefit (Di Veroli. C, Bioinformatics, 32(18), 2016, 2866-2868) is an interactive platform for the analysis and visualization of drug combinations and only requires the user to save the data in the predefined .xls template files. Combenefit performs combination analyses using the standard Loewe, Bliss and HSA methods. In the HSA model, the synergy score quantifies the excess over the highest single drug response. In the Loewe model, the synergy score quantifies the excess over the expected response if the two drugs are the same compound. In the Bliss model, the expected response is a multiplicative effect as if the two drugs act independently (see Ianevski et al., Bioinformatics, 2017, 33(15): 2413-2415).

The experimental dose-response surface that delineates combination effects in concentration space, is first read by the Combenefit software as a matrix of % of the control value across concentrations. Single agent effects are extracted from this data and fitted with a dose response curve. Based on the two single agent dose response curves, a model-based combination dose-response surface is derived. This surface provides a “reference” dose-response surface for a non-synergistic (additive/independent) combination, whose characteristics are determined by the selected model (i.e., Loewe, Bliss or HSA). The experimental combination dose response surface is then compared to the model-generated one, resulting in a synergy distribution in concentration space.

The graphical outputs consist of: (i) the single agent dose response data and its fitting; (ii) the combination dose response (four different displays); (iii) the model generated reference combination dose response, i.e., the prediction of effect if the drugs are not synergistic (three different displays); (iv) the resulting synergy distribution (three different displays); and (v) a graphic mapping the synergy distribution onto the dose-response surface.

This synergy distribution can be further summarized via various metrics (Di Veroli. C, Bioinformatics, 32(18), 2016, 2866-2868) as follows:
SYN_MAX—the maximum level of synergy observed.
SYN_SUM—the sum of synergy observed in concentration logarithmic space. For instance, an integrated synergy of 50 is equivalent to an extra synergistic effect of 50% which is spread over a square of 1 log×1 log in the 2-d log-concentration space.

Analysis of Combinations Using the Additive Model

Combination effects were also analyzed using the Additive model (see Valeriote et al., Cancer Chemother Rep. 1975, 59:895-900; Lepri et al., Hematol Oncol. 1991, 9:79-86; and Jonsson E et al., Eur J Clin Pharmacol. 1998, 54:509-14.) In samples with two co-incubated substances, a predicted cell viability (%) is calculated according to the following formula: Predicted cell viability (%)=cell viability of substance 1 (%)×cell viability of substance 2 (%)×0.01.

A “combination index” (CI) is then calculated as the measured cell viability of the sample with two co-incubated substances divided by the predicted cell viability. The following classifications are used in this example:
CI>1.2 sub-additive effect
CI=0.8−1.2 additive effect
CI<0.8 synergistic effect
CI<0.5 strong synergistic effect

If the measured cell viability for a combination of two substances is higher than the cell viability for one or both substances, the effect is considered antagonistic. If the predicted viability is very low, the quote “measured viability/predicted viability” may give false CI values. Thus, a lower limit of <5% of the predicted viability was set.
7.1.2 Results

As a single agent alone, both APR-246 and ABT-199 showed dose-dependent anti-proliferative activity with IC50 values ranging between 1-11 μM for APR-246 and 0.001-1.5 μM for ABT-199. IC50 values are summarized in Table 3.

TABLE 3
IC50 values (μM) for APR-246 and ABT-199

| Cell line | Cell type | P53 status | APR-246 | ABT-199 |
|---|---|---|---|---|
| MV4-11 | AML | R248W | 2.5 | 0.16 |
| KBM-3 | AML | WT | 3.7 | 1.36 |
| KG-1 | AML | Intron6/splice* | 11 | 1.5 |
| HL-60 | AML | Null (deletion) | 8.1 | 0.06 |
| MOLM-13 | AML | WT | 9.5 | 0.001 |
| SKM-1 | MDS | R248Q | 1.09 | 0.07 |

*The point mutation converts G into A in the splice donor site at the first base of the intron (c.672 + 1).

Synergy scores were calculated with the Combenefit software using three mathematical models (Bliss, Loewe, HSA) as described above. In addition, the Additive model was used to calculate a combination index for all combinations. Maximum synergy values and synergy sum for each model from Combenefit are summarized in Table 4.

TABLE 4
Maximum synergy values and synergy sum for each model
APR-246 + ABT-199

| SYN-MAX | | | | SYN-SUM | | |
|---|---|---|---|---|---|---|
| Cell line | LOEWE | BLISS | HSA | LOEWE | BLISS | HSA |
| KBM-3 | 33.9 | 37.1 | 38.9 | 17.9 | 19.0 | 24.7 |
| MV4-11 | 30.9 | 29.9 | 53.6 | 22.4 | 22.9 | 24.7 |
| KG-1 | 32.1 | 17.2 | 39.6 | 21.8 | 8.8 | 36.8 |
| HL-60 | 23.3 | 23.3 | 23.5 | 5.4 | 1.8 | 25.2 |
| MOLM-13 | 20.7 | 29.8 | 42.1 | 6.5 | 8.6 | 18.3 |
| SKM-1 | 23.2 | 16.8 | 37.7 | 21.5 | 12.3 | 28.5 |

Combination Index values for the individual cell lines with various doses of APR-246 and ABT-199 are shown in Tables 5 to 10 below (NC=not calculated).

TABLE 5
Combination index for KBM-3

| APR-246 | ABT-199 μM | | | | | | |
|---|---|---|---|---|---|---|---|
| μM | 0.39 | 0.78 | 1.56 | 3.13 | 6.25 | 12.50 | 25.00 |
| 1.32 | 0.99 | 0.92 | 0.95 | 0.88 | 0.88 | 0.89 | 0.85 |
| 1.98 | 0.95 | 0.91 | 0.90 | 0.80 | 0.80 | 0.73 | 0.68 |
| 2.96 | 0.66 | 0.58 | 0.60 | 0.42 | 0.44 | 0.26 | 0.14 |
| 4.44 | 0.08 | 0.17 | 0.02 | 0.02 | 0.01 | 0.03 | 0.05 |
| 6.67 | N.C | N.C | N.C | N.C | N.C | N.C | N.C |
| 10.00 | N.C | N.C | N.C | N.C | N.C | N.C | N.C |

combination Index (KBM-3)

TABLE 6
Combination index for KG-1

| APR-246 | ABT-199 μM | | | | | |
|---|---|---|---|---|---|---|
| μM | 0.22 | 0.44 | 0.88 | 1.75 | 3.50 | 7.00 |
| 2.19 | 1.23 | 1.28 | 1.01 | 1.41 | 1.16 | 0.99 |
| 3.29 | 1.00 | 0.94 | 0.93 | 1.08 | 0.92 | 0.81 |
| 4.94 | 0.24 | 0.71 | 0.74 | 0.70 | 0.63 | 1.24 |
| 7.41 | 1.19 | 0.85 | 0.86 | 1.22 | 1.07 | 0.51 |
| 11.11 | NC | NC | NC | NC | NC | NC |
| 16.67 | 0.88 | 1.05 | 1.07 | 1.10 | 0.99 | 0.92 |
| 25.00 | 0.90 | 1.09 | 1.16 | 1.73 | 1.66 | NC |

combination Index (KG-1)

TABLE 7
Combination index for HL-60

| APR-246 | ABT-199 μM | | | | |
|---|---|---|---|---|---|
| μM | 0.03 | 0.06 | 0.13 | 0.50 | 1.00 |
| 1.76 | 1.10 | 1.22 | 1.54 | 1.44 | 1.16 |
| 2.63 | 0.82 | 1.63 | 1.49 | 1.52 | 1.63 |
| 3.95 | 1.07 | 1.35 | 1.39 | 1.51 | 1.24 |
| 5.93 | 1.00 | 1.40 | 1.42 | 1.41 | 1.39 |
| 8.89 | 0.92 | 0.95 | 1.52 | 1.46 | 1.17 |
| 13.33 | 0.93 | 1.06 | 0.94 | NC | NC |
| 20.00 | NC | NC | NC | NC | NC |

combination Index (HL-60)

TABLE 8
Combination index for MOLM-13

| APR-246 | ABT-199 nM | | | | | | | |
|---|---|---|---|---|---|---|---|---|
| μM | 0.2 | 0.4 | 0.8 | 1.6 | 3.2 | 6.3 | 12.5 | 25.0 |
| 2.19 | 0.88 | 0.92 | 0.93 | 1.02 | 0.96 | 0.97 | 1.02 | NC |
| 3.29 | 0.83 | 0.78 | 0.89 | 0.93 | 0.96 | 0.95 | 0.78 | NC |
| 4.44 | 0.67 | 1.05 | 0.98 | 0.93 | 0.72 | 0.51 | NC | NC |
| 7.41 | 0.48 | 0.75 | 0.31 | 0.71 | 0.42 | 0.00 | NC | NC |
| 11.11 | NC | NC | NC | NC | NC | NC | NC | NC |

combination Index (Molm-13)

TABLE 9
Combination index for MV4-11

| | ABT-199 μM | | | | | | | | | | |
|---|---|---|---|---|---|---|---|---|---|---|---|
| APR-246 μM | 0.005 | 0.01 | 0.02 | 0.04 | 0.08 | 0.16 | 0.31 | 0.63 | 1.25 | 2.5 | 5 |
| 0.5 | 1.13 | 1.07 | 1.08 | 1.11 | 1.04 | 1.03 | 0.91 | NC | NC | NC | NC |
| 1.5 | 0.97 | 0.97 | 0.63 | 0.54 | 0.49 | 0.07 | 0.02 | NC | NC | NC | NC |
| 2.5 | 0.50 | 0.11 | 0.03 | 0.09 | 0.01 | 0.00 | 0.00 | NC | NC | NC | NC |
| 3.5 | 0.01 | 0.00 | 0.00 | 0.00 | 0.00 | 0.00 | NC | NC | NC | NC | NC |
| 4.5 | NC | NC | NC | NC | NC | NC | NC | NC | NC | NC | NC |
| 5 | NC | NC | NC | NC | NC | NC | NC | NC | NC | NC | NC |

combination Index (MV4-11)

TABLE 10
Combination index for SKM-1

| APR-246 | ABT-199 μM | | | | | | | | |
|---|---|---|---|---|---|---|---|---|---|
| μM | 0.04 | 0.08 | 0.16 | 0.31 | 0.63 | 1.25 | 2.5 | 5.0 | 10.0 |
| 0.50 | 0.87 | 0.65 | 0.72 | 0.65 | 0.35 | 0.48 | 0.44 | 0.36 | 0.20 |
| 1.00 | 0.50 | 0.20 | 0.15 | 0.14 | 0.09 | 0.06 | 0.14 | 0.10 | NC |
| 1.25 | 0.32 | 0.06 | 0.05 | 0.27 | 0.11 | 0.04 | 0.08 | NC | NC |
| 1.50 | 0.17 | 0.07 | 0.06 | 0.03 | 0.04 | 0.04 | 0.08 | NC | NC |
| 2.50 | NC | NC | NC | NC | NC | NC | NC | NC | NC |

combination Index (SKM-1)

Thus, in KBM3, MV4-11, KG-1, MOLM-13, SKM-1 the combination of APR-246 and ABT-199 showed synergy using HSA, Bliss, Loewe and Additive reference models.

Combination Index values corresponding to synergy and strong synergy were obtained in all these 5 cell lines. The synergy was apparent in APR-246 concentrations ranging from 2.96 to 4.5 μM for KBM3, 1.5 to 3.5 μM for MV4-11 and 3.5 to 11 μM for KG-1 cell line, 3.2 to 7.4 μM for MOLM-13, 0.5 to 1.5 μM for SKM-1. ABT-199 concentrations ranging from 0.3 to 25 μM for KBM3, 0.006 to 0.16 μM for MV4-11, 0.2 to 7 μM for KG-1, 0.2 to 6.2 nM for MOLM-13, 0.03 to 1.25 μM for SKM1.

In contrast, the HL-60 cell line (null p53) showed lower synergy sum scores than the other cell lines, and there was no Combination Index result of synergy or strong synergy. An antagonistic effect appeared in some dose combinations for this cell line and several Combination Index results of sub-additive effect were observed

### 7.2 Example 2

### Phase MI Study of APR-246 in Combination With Venetoclax and Azacitidine in TP53-Mutant Myeloid Malignancies

This clinical trial is a Phase I/II, open-label, dose-finding and cohort expansion study to determine the safety and preliminary efficacy of APR-246 in combination with venetoclax and azacitidine in patients with myeloid malignancies.

The study assesses (i) the clinical activity of APR-246 in combination with venetoclax and azacitidine in patients with myeloid malignancies by determination of complete remission (CR) rate; (ii) the clinical activity of APR-246 in combination with venetoclax and azacitidine in patients with myeloid malignancies by determination of overall response rate (ORR); (iii) the clinical activity of APR-246 in combination with venetoclax and azacitidine in patients with AML, by determination of progression-free survival (PFS); (iv) the clinical activity of APR-246 in combination with venetoclax and azacitidine in patients with AML by determination of transition rate to hematopoietic stem cell transplant (HSCT); (v) the clinical activity of APR-246 in combination with venetoclax and azacitidine in patients with AML by determination of overall survival (OS); and (vi) the pharmacokinetic profile of APR-246 and venetoclax and azacitidine.
7.2.1 Study Design

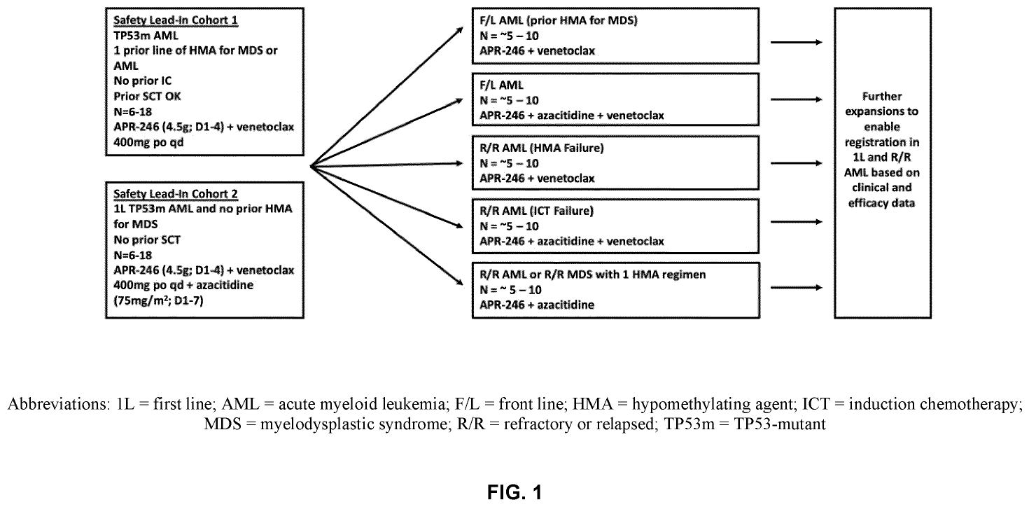

The study includes a safety lead-in dose finding portion followed by expansion portion (see FIG. 1). During the safety lead-in portion of the study, two parallel cohorts independently enroll patients following a 3+3 design as shown in the table below:

TABLE 11
Safety Lead-In Cohorts

| Cohorts | Patients | Treatments |
|---|---|---|
| Safety | patients with TP53-mutant AML | APR-246 + venetoclax |
| Lead-In | who have received no more than | |
| Cohort 1 | one prior hypomethylating agent | |
| | (HMA) regimen | |
| Safety | patients with previously untreated | APR-246 + venetoclax + |
| Lead-In | TP53-mutant acute myeloid | azacitidine |
| Cohort 2 | leukemia AML (and no prior | |
| | HMA for MDS) | |

More specifically, safety lead-in cohort 1 enrolls patients with AML or MDS who have received no more than one prior HMA regimen. These patients receive APR-246 at 4.5 g/day on days 1-4 of each 28-day cycle administered concurrently with venetoclax that is given orally at the dose of 400 mg daily once the ramp-up phase is completed. If a prophylactic antifungal agent is administered concurrently, the suggested daily dose of venetoclax is 200 mg. Safety lead-in cohort 2 enrolls patients with previously untreated TP53-mutant AML (prior MDS is allowed, but not treatment with HMA), who receive APR-246 at 4.5 g/day on days 1-4 administered concurrently with venetoclax given orally at the dose of 400 mg daily and with azacitidine at the standard dose of 75 mg/m² over 7 consecutive days as a subcutaneous injection or IV infusion on days 1-7 of each 28-day cycle.

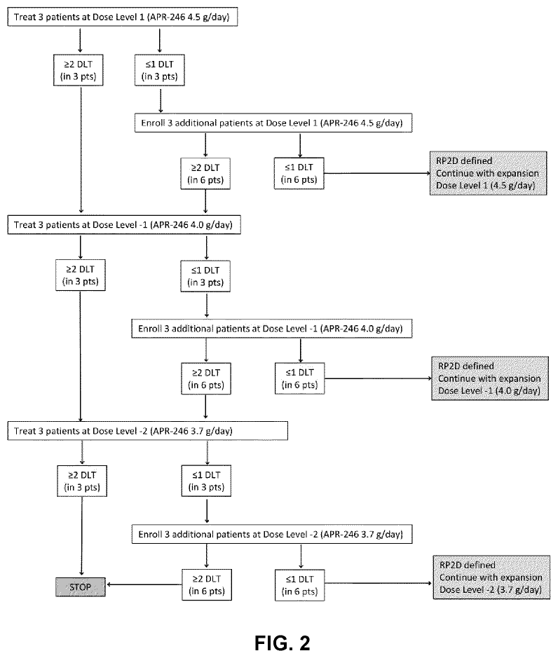

Dose-finding study design is applied to each cohort of the safety lead-in portion (see FIG. 2). Each cohort enrolls up to a maximum of 6 patients. Dose-limiting toxicity (DLT) is assessed after three patients have been enrolled in respective cohort and the last enrolled patient has completed the 4-week safety assessment period (i.e., one cycle of combination regimen). A patient that discontinues therapy during cycle 1 without DLT is considered evaluable for the purpose of safety only if at least 80% of scheduled doses of APR-246 in combination with venetoclax with and without azacitidine were administered in the first cycle. At the first dose level of 4.5 g/day of APR-246, if 1 or less than 1 patient out of 3 experiences a DLT, 3 additional patients are enrolled. If 1 or less than 1 patient out of 6 experiences DLT, the dose level (4.5 g/day of APR-246) is deemed the recommended Phase II dose (RP2D) for that cohort. If 2 or more than 2 patients out of the total 3-6 patients in the cohort experience DLT, the study continues enrollment at Dose Level −1 (4.0 g/day of APR-246). If 1 or less than 1 patient out of 6 experiences DLT at this dose level, the dose level (4.0 g/day of APR-246) is deemed the RP2D for that cohort. If 2 or more than 2 patients out of the total 3-6 patients at that dose level experience DLT, the study continues enrollment at Dose Level −2 (3.7 g/day of APR-246). If 1 or less than 1 patient out of 6 experiences DLT at this dose level, the dose level (3.7 g/day of APR-246) is deemed the RP2D for that cohort. If 2 or more than 2 patients out of the total 3-6 patients at this dose level experience DLT, no additional patients are enrolled at this dose. The trial is halted, and the DRT considers potential future dosing modifications. Dose modification is summarized below.

TABLE 12
Dose Modification for APR-246

| Dose | |
|---|---|
| Modification | APR-246 Dose |
| Starting Dose | APR-246 4.5 g/day |
| Level (DL) | 1.5 g (for first 45 minutes) + 3.0 g |
| | (for 5 hours 15 minutes) |
| Dose Level | APR-246 4.0 g/day |
| Reduction-1 | 1.33 g (for first 45 minutes) + 2.67 g |
| (DL-1) | (for 5 hours 15 minutes) |
| Dose Level | APR-246 3.7 g/day |
| Reduction-2 | 1.23 g (for first 45 minutes) + 2.47 g |
| (DL-2) | (for 5 hours 15 minutes) |

The expansion portion begins once the recommended Phase II dose (RP2D) of APR-246 in combination with venetoclax and in combination with venetoclax and azacitidine has been determined in order to assess the antitumor activity of these combinations. Up to 50 patientsare enrolled in five cohorts of patients with TP53-mutant myeloid malignancies:

TABLE 13
Expansion Cohorts

| Expansion | | |
|---|---|---|
| Cohorts | Treatments | Patients |
| Expansion | APR-246 + | Patients with newly diagnosed AML |
| Cohort 1 | venetoclax | arising from myelodysplastic syndrome |
| (n = 5-10) | | (MDS) previously treated with HMA |
| | | therapy, and no prior venetoclax or APR- |
| | | 246. Patients may have had prior SCT for |
| | | MDS. |
| Expansion | APR-246 + | Patients with newly diagnosed AML, no |
| Cohort 2 | venetoclax + | prior treatment for AML or MDS, |
| (n = 5-10) | azacitidine | including APR-246, venetoclax, HMA, |
| | | or SCT. |
| Expansion | APR-246 + | Patients with R/R AML who have |
| Cohort 3 | venetoclax | previously failed at least one HMA- |
| (n = 5-10) | | containing regimen. Prior venetoclax and |
| | | prior SCT are allowed. Prior exposure to |
| | | APR-246 is exclusionary. |
| Expansion | APR-246 + | Patients with AML that is primary |
| Cohort 4 | venetoclax + | refractory or are in their first relapse |
| (n = 5-10) | azacitidine | following intensive induction |
| | | chemotherapy, and no prior HMA, |
| | | venetoclax or APR-246. Patients may |
| | | have had prior SCT. |
| Expansion | APR-246 + | Patients with R/R AML or R/R MDS |
| Cohort 5 | azacitidine | following one HMA-containing regimen. |
| (n = 5-10) | | Prior venetoclax is allowed. Prior |
| | | exposure to APR-246 is exclusionary. |

7.2.2 Study Endpoints

Primary endpoints of the study are as follows: (1) dose-limiting toxicities (DLTs), classified and graded according to the National Cancer Institute's Common Terminology Criteria for Adverse Events (NCI-CTCAE, version 5.0); (2) frequency of treatment-emergent adverse events (TEAEs), and serious adverse events (SAEs) related to APR-246 in combination with venetoclax and azacitidine during the trial; and (3) the RP2D of APR-246 (the dose at which less than 2 out of 6 patients experience dose-limiting toxicity during the safety assessment period).

Secondary endpoints of the study include: (1) complete remission rate, defined as the proportion of patients who achieve complete remission; (2) overall response rate (ORR), defined as the proportion of patients achieving complete remission (CR), complete remission with incomplete hematologic recovery (CRi), complete remission with incomplete platelet recovery (CRp), partial remission (PR) and morphologic leukemia-free state (MLFS) by the IWG 2003 criteria (APPENDIX I) (the CRh rate will also be determined and is defined as bone marrow blasts<5%, ANC>0.5×109/L and platelets>50×109/L); (3) overall survival (OS), measured from the date of initiating study treatment to the date of death. Patients who have not died by the analysis data cut-off date will be censored at their last date of contact; (4) progression-free survival (PFS), defined from the date of initiating study treatment to the date of disease progression or death as a result of any cause; (5) proportion of patients who transition to hematopoietic stem cell transplantation (HSCT); and (6) pharmacokinetic parameters: Cmax (maximum concentration), AUC (area under the curve), Vd and clearance (CL) of APR-246 and Tmax (time of maximum concentration), Cmax and AUC of venetoclax.

Exploratory molecular analyses may include, but are not limited to: TP53 VAF by NGS, p53 immunohistochemistry, mutations in other genes by NGS, and RNA expression.
7.2.3 Eligibility Criteria For Patients in Safety Lead-In Cohorts

Inclusion criteria for patients in safety lead-in cohorts include, among other things:

| 1. | Documented diagnosis of AML according to World Health Organization (WHO) | |
|---|---|---|
| | classification (≥20% blasts in bone marrow and/or peripheral blood). | |
| 2. | Adequate organ function as defined by the following laboratory values: | |
| | a. | Creatinine clearance >30 mL/min (by Cockcroft-Gault method), |
| | b. | Total serum bilirubin <1.5 × ULN unless due to Gilbert's syndrome, |
| | | leukemic organ involvement, hemolysis or considered an effect of regular |
| | | blood transfusions, |
| | c. | Alanine aminotransferase (ALT) and aspartate aminotransferase (AST) >3 × |
| | | ULN, unless due to leukemic organ involvement. |

| | |
|---|---|
| 3. | Age ≥18 years at the time of signing the informed consent form. |
| 4. | At least one TP53 mutation which is not benign or likely benign. |
| 5. | Eastern Cooperative Oncology Group (ECOG) performance status of 0, 1 or 2. |
| 6. | Projected life expectancy of ≥12 weeks. |
| 7. | Negative serum or urine pregnancy test prior to study treatment initiation in female |
| | patients of childbearing potential. |
| 8. | Females of childbearing potential and males with female partners of childbearing |
| | potential must be willing to use an effective form of contraception such as latex |
| | condom, hormonal birth control, intrauterine device or double barrier method |
| | during chemotherapy treatment and for at least six months thereafter. |

Exclusion criteria for patients in safety lead-in cohorts include:

| 1. | Prior treatment for TP53-mutant acute myeloid leukemia, including hematopoietic | |
|---|---|---|
| | stem cell transplantation (for Safety Lead-In Cohort 2 only). | |
| 2. | Known history of HIV or active hepatitis B or active hepatitis C infection. | |
| 3. | Any of the following cardiac abnormalities: | |
| | a. | Myocardial infarction within six months prior to registration; |
| | b. | New York Heart Association Class III or IV heart failure or known left |
| | | ventricular ejection fraction (LVEF) <40%; |
| | c. | A history of familial long QT syndrome; |
| | d. | Symptomatic atrial or ventricular arrhythmias not controlled by medications; |
| | e. | QTcF ≥470 msec, unless due to underlying bundle branch block and/or |
| | | pacemaker and with approval of the medical monitor. |

| | |
|---|---|
| 4. | Concomitant malignancies for which patients are receiving active therapy at the |
| | time of signing consent. For example, patients with adequately treated basal or |
| | squamous cell carcinoma of the skin, adequately treated carcinoma in situ (e.g. |
| | cervix), or breast cancer receiving adjuvant endocrine therapy may enroll |
| | irrespective of the time of diagnosis with Medical Monitor approval. |
| 5. | Known active CNS involvement from AML. A diagnostic lumbar puncture is not |
| | required in the absence of suspicion for CNS disease. |
| 6. | Malabsorption syndrome or other condition that precludes enteral route of |
| | administration. |
| 7. | Pregnancy or lactation. |
| 8. | Active uncontrolled systemic infection (viral, bacterial or fungal). |

7.2.4 Eligibility Criteria For Patients in Expansion Cohorts

Inclusion criteria for patients in expansion cohorts include:

| 1. | Newly diagnosed, or relapsed or refractory, AML according to World Health | |
|---|---|---|
| | Organization (WHO) classification (≥20% blasts in bone marrow and/or peripheral | |
| | blood), as specified by cohort: | |
| | a. | Newly diagnosed AML arising from MDS, previously treated with HMA |
| | | regimen, and no prior venetoclax or APR-246 (Note: patients may have had |
| | | prior SCT for MDS); |
| | b. | Newly diagnosed AML, with no prior treatment for AML or MDS, including |
| | | APR-246, venetoclax and HMA, or prior SCT; |
| | c. | AML that is relapsed or refractory to at least one HMA regimen (Note: no |
| | | prior APR-246; prior venetoclax is allowed; patients may have had prior |
| | | SCT); |
| | d. | AML that is primary refractory or relapsed following intensive induction |
| | | chemotherapy, with no prior HMA, venetoclax or APR-246 (Note: patients |
| | | may have had prior SCT); |
| | e. | AML or MDS that is relapsed or refractory following one HMA-containing |
| | | regimen (Note: no prior APR-246; prior venetoclax is allowed). |
| 2. | Adequate organ function as defined by the following laboratory values: | |
| | a. | Creatinine clearance >30 mL/min (by Cockcroft-Gault method), |
| | b. | Total serum bilirubin <1.5 × ULN unless due to Gilbert's syndrome, |
| | | leukemic organ involvement, hemolysis or considered an effect of regular |
| | | blood transfusions, |
| | c. | Alanine aminotransferase (ALT) and aspartate aminotransferase (AST) <3 × |
| | | ULN, unless due to leukemic organ involvement. |

| | |
|---|---|
| 3. | Age ≥18 years at the time of signing the informed consent form. |
| 4. | At least one TP53 mutation which is not benign or likely benign. |
| 5. | Eastern Cooperative Oncology Group (ECOG) performance status of 0, 1 or 2. |
| 6. | Projected life expectancy of ≥12 weeks. |
| 7. | Negative serum pregnancy test prior to study treatment initiation in female patients |
| | of childbearing potential. |
| 8. | Females of childbearing potential and males with female partners of childbearing |
| | potential must be willing to use an effective form of contraception such as latex |
| | condom, hormonal birth control, intrauterine device or double barrier method |
| | during chemotherapy treatment and for at least six months thereafter. |

Exclusion criteria for patients in expansion cohorts include:

| 1. | Known history of HIV or active hepatitis B or active hepatitis C infection. | |
|---|---|---|
| 2. | Any of the following cardiac abnormalities: | |
| | a. | Myocardial infarction within six months prior to registration, |
| | b. | New York Heart Association Class III or IV heart failure or known left |
| | | ventricular ejection fraction (LVEF) <40%, |
| | c. | A history of familial long QT syndrome, |
| | d. | Symptomatic atrial or ventricular arrhythmias not controlled by medications, |
| | e. | QTcF ≥470 msec, unless due to underlying bundle branch block and/or |
| | | pacemaker and with approval of the medical monitor. |

| | |
|---|---|
| 3. | Concomitant malignancies for which patients are receiving active therapy at the |
| | time of signing consent. For example, patients with adequately resected basal or |
| | squamous cell carcinoma of the skin, adequately resected carcinoma in situ (e.g. |
| | cervix), or breast cancer receiving adjuvant endocrine therapy may enroll |
| | irrespective of the time of diagnosis with Medical Monitor approval. |
| 4. | Prior exposure to anticancer therapies including chemotherapy, radiotherapy or |
| | other investigational therapy, including targeted small molecule agents within |
| | 14 days of the first day of study treatment or within 5 half-lives prior to first dose |
| | of study treatment. |
| 5. | Prior exposure to biologic agents (e.g. monoclonal antibodies) for anti-neoplastic |
| | intent within 14 days prior to first dose of study drug. |
| 6. | Known active CNS involvement from AML. A diagnostic lumbar puncture is not |
| | required in the absence of suspicion for CNS disease. |
| 7. | Malabsorption syndrome or other condition that precludes enteral route of |
| | administration. |
| 8. | Pregnancy or lactation. |
| 9. | Active uncontrolled systemic infection (viral, bacterial or fungal). |

7.2.5 Treatment and Follow-Up

Treatment is administered on an outpatient basis. No investigational or commercial agents or therapies other than those described below is administered with the intent to treat the patient's disease. Hydroxyurea is used for control of leukocytosis. The study includes a safety lead-in dose-finding portion followed by the expansion portion. During the safety lead-in portion of the study, two cohorts will independently enroll patients following a 3+3 design as described above in Section 7.2.1. Each cohort enrolls up to 6 patients. The expansion portion begins once the recommended Phase II dose (RP2D) of APR-246 in combination with venetoclax and in combination with venetoclax and azacitidine have been determined in order to assess the antitumor activity of these combinations. Up to 50 patients are enrolled in five cohorts of patients with TP53-mutant myeloid malignancies as explained in more detail in Section 7.2.1. In each portion, patients continue treatment as long as toxicity remains acceptable and the patient has not withdrawn consent. Response is assessed based on the International Working Group (IWG) AML, response criteria after every treatment cycle the first year, then every two cycles.

Patients are followed based on the study calendar. After a patient is removed or withdrawn from study treatment, the patient is followed until death. Off-treatment data on overall survival is updated every 6 months or until death, whichever occurs first. If a patient is removed from the study due to unacceptable adverse events, the event(s) is followed until resolution or stabilization of the adverse event. Patients who respond and discontinue study treatment for reasons other than PD have response assessments and survival is collected every month until relapse or death, whichever occurs first. After relapse, data for survival is collected every 6 months until death.

Study treatment continues for patients receiving clinical benefit, unless one or more withdrawal criteria are met, or at the patient's discretion, or if the study is terminated. Study treatment must be discontinued if (1) evidence of disease progression (patients who have relapsed or progressive disease but who are continuing to derive clinical benefit in the opinion of the investigator may continue to receive study treatment); (2) a patient becomes pregnant; (3) a patient is significantly non-compliant with the requirements of the protocol; (4) a patient has an adverse experience that would make continued participation in the study an unacceptable risk; (5) the patient starts new treatment for their underlying disease.
7.2.6 Statistics

The recommended Phase II dose (RP2D) of APR-246 is defined as the dose at which<2 out of 6 patients experience dose-limiting toxicity (DLT) during the 4-week safety assessment period after administration of APR-246 in combination with venetoclax and azacitidine.

Definition of Dose-Limiting Toxicity (DLT)

An event is considered a DLT per NCI CTCAE version 5.0 criteria if it occurs within the 4-week safety assessment period (Cycle 1 of study treatment) and is not attributable to the underlying disease. DLT is defined as: (1) absolute neutrophil count (ANC) not recovering to >0.5×109/L and/or platelets not recovering to >25×109/L by day 42 of C1D1 in the absence of active leukemia or myelodysplasia; (2) grade≥3 nausea/vomiting/diarrhea or CNS toxicity that does not resolve to Grade≤1 within 7 days despite treatment interruption and/or maximal medical therapy; and/or (3) treatment related non-hematological Grade≥3 toxicity that does not resolve to Grade≤1.

A DLT is considered related to the study treatment unless there is a clear, alternative explanation for the AE. A≥Grade 3 metabolic or electrolyte abnormalities that is not clinically significant and is adequately controlled within 72 hours is not to be considered DLT. Tumor lysis syndrome (TLS) that responds to therapy and resolves within 72 hours is not considered DLT. Additionally, AEs that meet the above criteria, but occur after the DLT evaluation period is not defined as DLTs, but is reported as AEs/Serious Adverse Events (SAEs) and is reviewed across all cohorts during the study to help determine the AE profile. A patient that discontinues therapy during Cycle 1 without DLT is considered evaluable for the purpose of safety only if at least 80% of scheduled Cycle 1 dose of APR-246 in combination with venetoclax and azacitidine were administered in the first cycle.

Data Review Team (DRT) consisting of the Medical Monitor, Site Principal Investigators, and other clinical research personnel that the Sponsor may deem appropriate, will hold Data Review Meetings (DRM) on an interim basis at a frequency dependent on study accrual. At these meetings, the DRT will review AEs and dose-limiting toxicities (DLT) and make recommendations regarding the recommended Phase II dose (RP2D).

Determination of Sample Size

This trial assumes a sample size of 12-36 patients (6-18 patients for each cohort) in the safety lead-in portion of the study and up to 50 patients in the expansion portion of the study.

Analysis Populations

Safety population: patients are evaluable for safety if they receive at least one dose of APR-246 with venetoclax and azacitidine. The safety population is used to summarize exposure and safety parameters.

DLT-evaluable population: all patients who either experienced a DLT during first the 4 weeks (Cycle 1) of the study treatment or received >80% of scheduled Cycle 1 dose of APR-246 in combination with venetoclax and azacitidine and did not experience a DLT. Any individual patient who is not evaluable for DLT is replaced by a new patient through additional patient enrollment.

Efficacy evaluable (EE) population: all patients who complete at least one treatment cycle of APR-246 in combination with venetoclax and azacitidine and who have at least one post-treatment clinical response assessment. Patients who fail to complete one treatment cycle are considered EE if they show clear evidence of clinically significant disease progression. The EE population is the secondary analysis population for efficacy.

Pharmacokinetics (PK) population: patients are evaluable for pharmacokinetics if at least one post-dose sample for PK evaluation has been obtained.

Efficacy Analysis

Complete remission (CR) is summarized for all enrolled patients as the proportion (%) of patients with CR. In addition to presenting the CR rate, its associated exact 95% confidence intervals (CI) for each treatment arm are also presented. CR rate is not formally compared between treatment arms.

Duration of response (DoR) is defined as the time from the date when criteria for response are met to the date of relapse or progressive disease (PD) or death due to any cause, whichever occurs first. Patients alive with no progressive disease have their DOR censored at the date of the last clinical assessment. The duration of CR is summarized in each treatment arm by providing duration of complete response (DOCR) and duration of overall response (DOR), using Kaplan-Meier methodology. DoR endpoints are not formally compared between treatment arms.

Overall response is summarized in number (%) of patients in each category of responses and ORR is analyzed by using the similar method as CR rate. Survival data are collected at treatment and follow-up periods. Patients are followed until death. Overall survival (OS) is defined as the number of days from the first day of treatment to the date of death. Kaplan-Meier methodology is utilized.

Progression-free survival (PFS) is defined as the time from the first day of treatment to disease progression or death from AML, whichever occurs first. If neither event occurs, PFSis censored at the date of the last clinical assessment. Kaplan-Meier methodology is utilized.

Safety Analysis

Safety data including adverse events, vital signs, laboratory data, ECG, physical exam are tabulated for the safety population. Adverse events are tabulated by System Organ Class (SOC), preferred term, severity, and relationship to treatments. The tabulation of laboratory parameters include the normal ranges for each parameter. Each value is classified as falling above, below, or within the normal range. Laboratory parameters are tabulated by maximum NCI-CTCAE v5.0 severity grade.

Pharmacokinetic Analysis

PK sampling for APR-246 are performed in Cycle 1, on Days 1, 2 and 4. PK sampling for venetoclax is performed on Days 1 and 21 of Cycle 1 and on Day 4 of Cycles 2-5. Non-compartmental or population pharmacokinetic methods are used to derive APR-246 and venetoclax PK parameters (Cmax, Tmax, AUC, Vd and CL).

The pharmacokinetics of APR-246 and venetoclax are summarized using descriptive statistics (mean, standard deviation, CV % mean, geometric mean, CV % geometric mean) and compared with historical control data.

APR-246 AUC and Cmax are then tested for association with signs of efficacy and safety. If an observable trend exists, a PK/PD model is developed to evaluate the exposure-response relationship between APR-246 plasma exposure and outcome measures in the presence of venetoclax. Demographic and clinical data (ethnicity, current age, body weight, sex, disease status, etc.) are utilized to assess interpatient variability in the PK and PK/PD relationships.

### 7.3 Example 3

### Synergistic Effects of Compound A in Combination With a Bcl-2 Inhibitor

Synergistic effects of another exemplary p53 reactivator (i.e., 2,2,2-trifluoro-N-((3-oxoquinuclidin-2-yl)methyl)acetamide or Compound A) in combination with an exemplary Bcl-2 inhibitor (i.e., ABT-199 or Venetoclax) were tested in AML cell lines MV4-11 and KBM-3. A matrix combinational dose response screening of Compound A and ABT-199 was performed similarly as described in Example 1 above. IC50 value for Compound A is shown in the table below.

TABLE 14
IC50 values (μM) for Compound A

| Cell line | Compound A |
|---|---|
| MV4-11 | 1.0 |
| KBM-3 | 1.7 |

The Additive model was used to calculate a combination index for all combinations of Compound A and ABT-199. For each cell line, at least one dose combination had a Combination Index fulfilling the definition of Strong Synergistic Effect (<0.5) according to the Additive Model (see Tables 15 and 16).

TABLE 15
Combination Index for Compound A and ABT-199 in MV4-11

| | ABT-199 μM | | | | | | | |
|---|---|---|---|---|---|---|---|---|
| | | 0.008 | 0.016 | 0.031 | 0.063 | 0.125 | 0.250 | 0.500 |
| Compound | 0.50 | 0.878 | 1.011 | 1.024 | 1.012 | 1.071 | 0.918 | 0.951 |
| A μM | 1.00 | 0.934 | 1.070 | 1.108 | 1.011 | 0.807 | 0.622 | 0.598 |
| | 1.50 | 0.518 | 0.788 | 0.287 | 0.152 | 0.143 | 0.039 | 0.013 |
| | 2.00 | 0.735 | 0.775 | 0.361 | 0.221 | 0.080 | 0.045 | 0.025 |
| | 2.50 | 1.388 | NC | NC | NC | NC | NC | NC |
| | 3.50 | NC | NC | NC | NC | NC | NC | NC |

Combination Index (MV4-11)

TABLE 16
Combination Index for Compound A and ABT-199 in KBM3

| | | ABT-199 μM | | | | | | | |
|---|---|---|---|---|---|---|---|---|---|
| | | 0.39 | 0.78 | 1.56 | 3.13 | 6.25 | 12.50 | 25.00 | 50.00 |
| Compound | 1.0 | 0.99 | 0.95 | 0.85 | 0.87 | 0.79 | 0.76 | 0.87 | 0.74 |
| A μM | 1.5 | 0.91 | 0.85 | 0.78 | 0.80 | 0.72 | 0.66 | 0.60 | 0.58 |
| | 2.0 | 0.79 | 0.59 | 0.54 | 0.54 | 0.41 | 0.33 | 0.29 | NC |
| | 2.5 | 0.32 | 0.18 | 0.35 | 0.06 | 0.01 | 0.01 | 0.02 | NC |
| | 3.0 | 0.34 | 0.04 | 0.01 | 0.02 | 0.00 | NC | NC | NC |
| | 4.0 | 0.00 | NC | NC | NC | NC | NC | NC | NC |

Combination Index (KBM-3)

In a separate study, a matrix combinational dose response screening of Compound A and ABT-199 was performed in MOLM-13 (an AML cell line) and SKM-1 (a MDS/AML cell line) to assess both single agent activity and evaluate additive, synergistic or antagonistic interactions across to a range of doses.

As a single agent, both Compound A and ABT-199 showed dose-dependent cytotoxic activity. IC₅₀ values are summarized in Table 17 below.

TABLE 17
IC₅₀ values (μM) for Compound A and ABT-199

| Cell line | Cell type | P53 status | Compound A | ABT-199 |
|---|---|---|---|---|
| SKM-1 | MDS/ | R248Q | 0.66 | 0.07 |
| | AML | (homo/ | | |
| | | hemizygous) | | |
| MOLM-13 | AML | WT | 3.6 | 0.0014 |

Synergy scores were calculated with the Combenefit software using three mathematical models (Bliss, Loewe, HSA) and in addition, the Additive model was used to calculate a combination index for all combinations. Maximum synergy values and synergy sum for each model are summarized in Table 18.

TABLE 18
SYN-MAX, SNY-SUM
Compound A + ABT-199

| SYN-MAX | | | | SYN-SUM | | |
|---|---|---|---|---|---|---|
| Cell line | LOEWE | BLISS | HSA | LOEWE | BLISS | HSA |
| SKM-1 | 19.8 | 18.8 | 31.9 | 17.5 | 14.3 | 28.2 |
| MOLM-13 | 11.9 | 12.4 | 20.6 | 2.9 | 2.4 | 9.1 |

In sum, in all AML cell lines KBM3, MV4-11, MOLM-13, and the MDS/AML cell line SKM-1, the combination of Compound A and ABT-199 was synergistic in the majority of cases using the Highest Single Agent (HSA), Bliss and Loewe models. Additive model Combination Index values corresponding to synergy and strong synergy were also obtained in these cell lines.

### 7.4 Example 4

### Synergistic Effects of APR-246 in Combination With Mcl-1 Inhibitors

Myeloid cell leukemia-1 (Mcl-1) is an antiapoptotic Bcl-2 family protein which is highly expressed in some cancers such as leukemia. Specific Mcl-1 inhibitors have been developed to target Mcl-1 and activate the Bax/Bak-dependent mitochondrial apoptotic pathway. In this study, the Mcl-1-inhibitors AMG-176 (Caenepeel et al., Cancer Discov, 8(12): 1-16 (2018)) and MIK665 (Adams and Cory, Cell Death and Differentiation, 25: 27-36 (2018)) were used to investigate their synergistic effects with APR-246 in AML cell lines. Synergistic effects were analysed using similar methods described in Section 0 above.
7.4.1 Results

As a single agent Mcl-1 inhibitors showed dose-dependent anti-proliferative activity with IC50 values ranging between 0.07-1.8 μM for AMG-176 and 0.001-0.6 μM for MIK665, as summarized in Table 19.

TABLE 19
IC50 values (μM) for AMG-176 and MIK665

| Cell line | AMG-176 | MIK665 |
|---|---|---|
| MV4-11 | 0.14 | 0.0014 |
| MOLM-13 | 0.07 | 0.002 |
| KBM-3 | 0.7 | 0.04 |
| KG-1 | 1.8 | 0.6 |
| HL-60 | 0.5 | 0.007 |

Synergy scores were calculated with the Combenefit software using three mathematical models (Bliss, Loewe, HSA) as described above. In addition, the Additive model was used to calculate a combination index for all combinations. Maximum synergy values and synergy sum for each model are summarized in Tables 20 and 21.

TABLE 20
Maximum synergy values for each model

| | APR-246 + AMG-176 | | | APR-246 + MIK-665 | | |
|---|---|---|---|---|---|---|
| | Synergy-MAX | | | | | |
| Cell line | LOEWE | BLISS | HSA | LOEWE | BLISS | HSA |
| KBM3 | 19.4 | 32.2 | 48.2 | 42.6 | 52.0 | 57.7 |
| MV4-11 | 13.5 | 18.4 | 38.7 | 18.2 | 24.3 | 48.5 |
| KG-1 | 11.5 | 24.6 | 37.0 | 17.9 | 17.8 | 32.0 |
| MOLM-13 | 23.4 | 30.7 | 42.3 | 15.8 | 16.1 | 34.1 |
| HL-60 | 3.5 | 7.2 | 20.4 | 31.1 | 64.3 | 68.8 |

TABLE 21
Synergy sum for each model

| | APR-246 + AMG-176 | | | APR-246 + MIK-665 | | |
|---|---|---|---|---|---|---|
| | Synergy-SUM | | | | | |
| Cell line | LOEWE | BLISS | HSA | LOEWE | BLISS | HSA |
| KBM3 | 8.0 | 18.9 | 26.2 | 20.4 | 33.1 | 39.0 |
| MV4-11 | 3.6 | 9.7 | 15.0 | 3.7 | 6.9 | 10.5 |
| KG-1 | 7.0 | 5.7 | 14.8 | 7.4 | 5.2 | 15.0 |
| MOLM-13 | 5.0 | 9.1 | 15.8 | 4.9 | 8.0 | 15.0 |
| HL-60 | 0.7 | 1.5 | 3.3 | 13.0 | 25.7 | 27.5 |

Combination Index values for the individual cell lines and combinations are shown in Tables 22-31 below (NC=not calculated).

TABLE 22
Combination Index for APR-246 and AMG-176 in KBM3

| | | AMG-176 μM | | | | | | | |
|---|---|---|---|---|---|---|---|---|---|
| | | 0.05 | 0.09 | 0.19 | 0.38 | 0.75 | 1.50 | 3.00 | 6.00 |
| APR246 μM | 1.32 | 0.94 | 1.09 | 1.00 | 1.06 | 0.99 | 0.83 | 0.19 | 0.00 |
| | 1.98 | 0.83 | 0.93 | 0.84 | 0.81 | 0.63 | 0.33 | 0.00 | 0.00 |
| | 2.96 | 0.85 | 0.88 | 0.83 | 0.69 | 0.27 | 0.00 | N.C | N.C |
| | 4.44 | 0.76 | 0.78 | 0.27 | 0.01 | 0.01 | 0.00 | N.C | N.C |
| | 6.67 | N.C | N.C | N.C | N.C | N.C | N.C | N.C | N.C |

Combination Index (KBM3)

TABLE 23
Combination Index for APR-246 and MIK665 in KBM3

| | | MIK665 μM | | | | | | | |
|---|---|---|---|---|---|---|---|---|---|
| | | 0.00 | 0.01 | 0.02 | 0.03 | 0.06 | 0.13 | 0.25 | 0.50 |
| APR246 μM | 1.32 | 1.23 | 0.97 | 1.07 | 1.26 | 1.09 | 0.47 | 0.01 | N.C |
| | 1.98 | 1.00 | 0.63 | 0.62 | 0.48 | 0.42 | 0.03 | 0.00 | N.C |
| | 2.96 | 0.82 | 0.74 | 0.29 | 0.11 | 0.02 | 0.00 | 0.00 | N.C |
| | 4.44 | 0.03 | 0.15 | 0.00 | 0.00 | 0.00 | 0.00 | 0.00 | N.C |
| | 6.67 | N.C | N.C | N.C | N.C | N.C | N.C | N.C | N.C |

Combination Index (KBM3)

TABLE 24
Combination Index for APR-246 and AMG-176 in MV4-11

| | | AMG-176 μM | | | | | | | | |
|---|---|---|---|---|---|---|---|---|---|---|
| | | 0.03 | 0.04 | 0.07 | 0.10 | 0.15 | 0.22 | 0.33 | 0.50 | 0.75 |
| APR246 μM | 0.50 | 0.96 | 1.09 | 0.82 | 1.24 | 0.69 | 0.63 | N.C | N.C | N.C |
| | 1.50 | 0.86 | 0.69 | 0.66 | 0.57 | 0.01 | N.C | N.C | N.C | N.C |
| | 2.50 | 0.71 | 0.48 | 0.24 | 0.00 | 0.00 | N.C | N.C | N.C | N.C |
| | 3.50 | 0.57 | 0.01 | 0.01 | N.C | N.C | N.C | N.C | N.C | N.C |
| | 4.50 | N.C | N.C | N.C | N.C | N.C | N.C | N.C | N.C | N.C |

Combination Index (MV4-11)

TABLE 25
Combination Index for APR-246 and MIK665 in MV4-11

| | | MIK665 μM | | | | |
|---|---|---|---|---|---|---|
| | | 0.001 | 0.002 | 0.003 | 0.004 | 0.006 |
| APR246 μM | 0.5 | 1.01 | 1.12 | 0.85 | 0.36 | 0.13 |
| | 1.5 | 0.44 | 0.29 | 0.01 | 0.00 | N.C |
| | 2.5 | 0.01 | 0.00 | 0.00 | 0.00 | N.C |
| | 3.5 | 0.00 | N.C | N.C | N.C | N.C |

Combination Index (MV4-11)

TABLE 26
Combination Index for APR-246 and AMG-176 in KG-1

| | | AMG-176 μM | | | | | | |
|---|---|---|---|---|---|---|---|---|
| | | 0.47 | 0.70 | 1.05 | 1.58 | 2.37 | 3.56 | 5.33 |
| APR246 μM | 1.98 | 0.89 | 0.79 | 0.50 | 0.71 | 0.56 | 0.42 | 0.21 |
| | 2.96 | 0.80 | 0.63 | 0.52 | 0.50 | 0.24 | 0.32 | N.C |
| | 4.44 | 0.67 | 0.89 | 0.54 | 0.68 | 0.29 | N.C | N.C |
| | 6.67 | 1.10 | 0.82 | 0.79 | 0.87 | 1.07 | N.C | N.C |
| | 10.00 | 1.12 | 1.00 | 1.16 | 1.17 | 1.24 | N.C | N.C |

Combination Index (KG1)

TABLE 27
Combination Index for APR-246 and MIK665 in KG-1

| | | MIK665 μM | | | | | | |
|---|---|---|---|---|---|---|---|---|
| | | 0.18 | 0.26 | 0.40 | 0.59 | 0.89 | 1.33 | 2.00 |
| APR246 μM | 1.98 | 0.98 | 1.03 | 0.91 | 0.97 | 1.12 | 0.63 | 0.33 |
| | 2.96 | 0.61 | 0.63 | 0.82 | 0.99 | 0.98 | 0.24 | 0.40 |
| | 4.44 | 0.73 | 0.55 | 0.28 | 0.77 | 0.51 | 0.28 | 0.30 |
| | 6.67 | 0.86 | 0.92 | 0.78 | 0.93 | 1.13 | N.C | N.C |
| | 10.00 | 1.12 | 1.37 | 1.37 | 1.26 | 1.44 | 1.32 | N.C |
| | 15.00 | 0.61 | 0.58 | 0.61 | 0.73 | 0.89 | N.C | N.C |

Combination Index (KG1)

TABLE 28
Combination Index for APR-246 and AMG-176 in HL-60

| | | AMG-176 μM | | | | |
|---|---|---|---|---|---|---|
| | | 0.29 | 0.44 | 0.66 | 0.99 | 1.48 |
| APR246 μM | 1.98 | 1.13 | 1.28 | 0.99 | 0.99 | N.C |
| | 2.96 | 1.13 | 1.13 | 1.30 | 1.30 | N.C |
| | 4.44 | 0.90 | 1.02 | 1.02 | 0.99 | N.C |
| | 6.67 | 1.01 | 0.80 | 0.80 | 0.60 | N.C |
| | 10.00 | 1.20 | 0.91 | 0.50 | 0.08 | N.C |
| | 15.00 | N.C | N.C | N.C | N.C | N.C |

Combination Index (HL-60)

TABLE 29
Combination Index for APR-246 and MIK665 in HL-60

| | | MIK665 μM | | | | | | |
|---|---|---|---|---|---|---|---|---|
| | | 0.002 | 0.003 | 0.013 | 0.025 | 0.050 | 0.100 | 0.200 |
| APR246 μM | 1.975 | 1.174 | 0.960 | 1.128 | 0.773 | 0.642 | 0.426 | N.C |
| | 2.963 | 1.239 | 0.918 | 1.095 | 0.606 | 0.493 | 0.302 | N.C |
| | 4.444 | 0.948 | 0.791 | 0.821 | 0.343 | 0.184 | 0.189 | N.C |
| | 6.667 | 0.956 | 0.812 | 0.538 | 0.123 | 0.083 | 0.029 | N.C |
| | 10.000 | 1.083 | 0.860 | 0.236 | 0.021 | 0.009 | 0.038 | N.C |
| | 15.000 | 0.573 | 0.641 | 0.025 | 0.013 | 0.050 | N.C | N.C |

Combination Index (HL-60)

TABLE 30
Combination Index for APR-246 and AMG-176 in MOLM-13

| | | MIK665 μM | | | | | | |
|---|---|---|---|---|---|---|---|---|
| | | 0.002 | 0.003 | 0.013 | 0.025 | 0.050 | 0.100 | 0.200 |
| APR246 μM | 1.975 | 1.174 | 0.960 | 1.128 | 0.773 | 0.642 | 0.426 | N.C |
| | 2.963 | 1.239 | 0.918 | 1.095 | 0.606 | 0.493 | 0.302 | N.C |
| | 4.444 | 0.948 | 0.791 | 0.821 | 0.343 | 0.184 | 0.189 | N.C |
| | 6.667 | 0.956 | 0.812 | 0.538 | 0.123 | 0.083 | 0.029 | N.C |
| | 10.000 | 1.083 | 0.860 | 0.236 | 0.021 | 0.009 | 0.038 | N.C |
| | 15.000 | 0.573 | 0.641 | 0.025 | 0.013 | 0.050 | N.C | N.C |

Combination Index (HL-60)

TABLE 31
Combination Index for APR-246 and MIK665 in MOLM-13

| | | MIK-665 μM | | | | | |
|---|---|---|---|---|---|---|---|
| | | 0.001 | 0.002 | 0.003 | 0.006 | 0.013 | 0.025 |
| APR246 μM | 2.63 | 1.22 | 1.14 | 0.90 | 0.61 | N.C | N.C |
| | 3.95 | 1.01 | 1.00 | 0.77 | 0.23 | N.C | N.C |
| | 5.93 | 0.92 | 1.03 | 0.64 | 0.12 | N.C | N.C |
| | 8.89 | 0.97 | 0.85 | 0.29 | 0.00 | N.C | N.C |
| | 13.33 | 0.75 | 0.38 | 0.00 | 0.00 | N.C | N.C |
| | 20.00 | N.C | N.C | N.C | N.C | N.C | N.C |

Combination Index (MOLM-13)

Thus, in KBM3, MV4-11, KG-1 and MOLM-13 the combination of APR-246 and either of both Mcl-1 inhibitors showed synergy using HSA, Bliss, Loewe and Additive reference models. The HL-60 cell line (null p53) showed lower synergy-max and synergy-sum scores with AMG-176 than the other cell lines, and an antagonistic effect appeared in some dose combinations. There were also fewer Combination Index results of synergy or strong synergy. However, with MIK665 similar synergistic effects was were obtained.

### 7.5 Example 5

### A Phase I/II and Dose Expansion Study of APR-246 in Combination With Venetoclax and Rituximab in Subject With TP53-Mutant Relapsed and/or Refractory Non-Hodgkin Lymphoma (NHL)

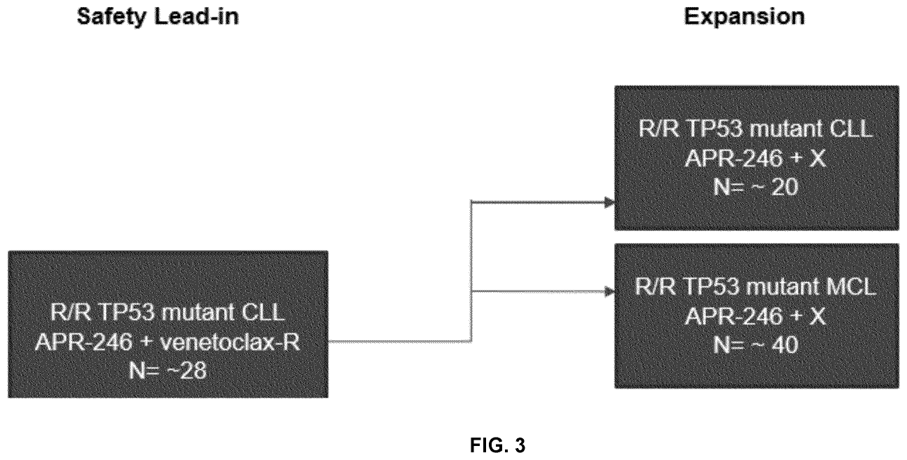

This example relates a Phase I/II, open-label, dose-finding and cohort expansion study to determine the preliminary safety, tolerability, and pharmacokinetic (PK) profile of APR-246 in combination with venetoclax and rituximab therapy in subjects with TP53-mutant NHL, including relapsed and/or refractory (R/R) CLL and R/R MCL. The study includes a safety lead-in dose de-escalation portion in subjects with R/R CLL. Once the safety, tolerability, and PK of APR-246 in combination with venetoclax and rituximab is established, an expansion portion proceeds with APR-246 in combination with venetoclax and rituximab therapy in subjects with R/R CLL and R/R MCL. The study design is described in FIG. 3.
7.5.1 Study Design
Safety Lead-In Study

In the safety lead-in portion of the study to determine the Recommended Phase II dose (RP2D), a safety lead-in cohort enrolls subjects with TP53-mutant CLL in a 3+3 dose de-escalation design: APR-246+venetoclax+rituximab (N≤28), in subjects with no prior Bcl-2 inhibitor therapy (e.g., venetoclax).

In safety lead-in cohort, patients start treatment with venetoclax as a single agent for the 5-week ramp-up period prior to Cycle 1 Day 1. Venetoclax is given PO at the dose of 20 mg during Week 1, 50 mg during Week 2, 100 mg during Week 3, 200 mg during Week 4, and 400 mg during Week 5. After the 5-week ramp-up, Cycle 1 treatment consists of APR 246 at 4.5 g/day IV on Days 1-4 administered concurrently with venetoclax at 400 mg (or tolerable dose following 5-week ramp up) PO daily. IV rituximab is initiated at 375 mg/m² on Cycle 1, Day 5 and 500 mg/m2 on Day 5 of Cycles 2-6. After 6 cycles of treatment with APR-246 in combination with venetoclax+rituximab, APR-246+venetoclax continue to be administered, starting at Cycle 7 to patients enrolled in the safety lead-in portion of the study for up to 24 cycles.

The safety lead-in cohort initially enrolls 3 subjects. Subjects are assessed for dose limiting toxicity (DLT) related to APR-246 with venetoclax and rituximab combination therapy after the first 3 subjects are enrolled in respective cohorts and the last enrolled subject has completed the 4-week safety assessment period (i.e., one cycle of combination regimen).

The does de-escalation is as follows:
The safety lead-in cohort initially enrolls 3 subjects
If≤1 subject out of 3 experiences a dose limiting toxicity (DLT), 3 additional subjects are recruited and treated at the same dose level (4.5 g/day of APR-246 on Days 1-4 of each 28-day cycle).
If≥2 subjects out of 3-6 subjects in a cohort experience a DLT, the study continues enrollment of 3 additional subjects at Dose Level −1 (4.0 g/day of APR-246 on Days 1-4 of each 28-day cycle).
If>1 subject out of 3 experience DLT, no additional subjects is recruited and treated at the same dose level. If≤1 subject out of 3 experiences DLT at the reduced dose of 4.0 g/day of APR-246 in the first 28 days of Cycle 1, 3 additional subjects are recruited and treated at the same dose level.
If≥2 subjects out of the total 3-6 subjects in the cohort of Dose Level−1 experience DLT, the study continues enrollment of 3 additional subjects at Dose Level−2 (3.5 g/day of APR-246 on Days 1-4 of each 28-day cycle).
If≤1 subject out of 3 experiences a DLT, 3 additional subjects are recruited and treated at the same dose level (3.5 g/day of APR-246 on Days 1-4 of each 28-day cycle).
If≥2 subjects of the total 3-6 subjects in the cohort experience DLT, the study is temporarily discontinue enrollment and the Data Review Team consider further enrollment and possible dose/schedule adjustments.

Dose modification is summarized in the Table 32 below.

TABLE 32
Dose Modification

| Dose Modification | APR-246 Dose |
|---|---|
| Starting Dose Level | APR-246 4.5 g/day |
| (DL) | 1.5 g (for first 45 minutes) + 3.0 g |
| | (for 5 hours 15 minutes) |
| Dose Level | APR-246 4.0 g/day |
| Reduction-1 (DL-1) | 1.33 g (for first 45 minutes) + 2.67 g |
| | (for 5 hours 15 minutes) |
| Dose Level | APR-246 3.5 g/day |
| Reduction-2 (DL-2) | 1.16 g (for first 45 minutes) + 2.34 g |
| | (for 5 hours 15 minutes) |

Expansion Study

The expansion portion begins once preliminary safety, tolerability, and PK of APR-246 in combination with venetoclax+rituximab has been established. Expansion proceeds with APR-246 in combination with venetoclax+rituximab therapy in subjects with R/R CLL and R/R MCL. In expansion, up to 60 subjects are enrolled and stratified into one of two cohorts: (1) Expansion Cohort 1: subjects with TP53-mutant R/R CLL (N≤20); and (2) Expansion Cohort 2: subjects with TP53-mutant R/R MCL (N≤40).

In Expansion Cohort 1, patients with R/R CLL receive APR-246 at the RP2D identified in Safety lead-in study of APR-246 with venetoclax+rituximab based on safety, PK, and/or preliminary efficacy data in the Safety lead-in cohort.

In Expansion Cohort 2, patients with R/R MCL receive APR-246 at the RP2D identified in Safety lead-in study of APR-246 with venetoclax +rituximab, based on safety, PK, and/or preliminary efficacy data in the Safety lead-in cohort.

Responses of CLL subjects are assessed according to disease specific response criteria for R/R CLL, e.g., the International Workshop on Chronic Lymphocytic Leukemia (iwCLL) 2018 guidelines for response assessment of CLL via blood, bone marrow (BM), and imaging (computed tomography [CT]), as appropriate, on Day 1 of Cycles 4, 7, 13, 19, and 25 (Hallek, M., et al., Blood 131(25), 2745-2760 (2018)). Responses and PD of CLL subjects are assessed according to Revised Criteria for Response Assessment of Hodgkin's and Non-Hodgkin's Lymphoma (Lugano Criteria) via blood, bone marrow (BM), and imaging (positron emission tomography [PET]/CT), as appropriate, on Day 1 of Cycles 4, 7, 13, 19, and 25 (van Heertum, R. L., et al., Drug. Des. Devel. Ther. 11, 1719-1728 (2017)). If IV contrast is contraindicated, CT without contrast can be used. For patients with bone marrow (BM) disease, BM assessments are done at Day 1 of Cycle 7, 13 and 25 months and if required to confirm complete response. For patients who achieve complete remission, imaging may be omitted and MRD assessment are completed using flow cytometry and/or molecular techniques.

In each portion of the study, subjects may continue treatment as long as toxicity remains acceptable and the subject has not withdrawn consent.

Further study of APR-246 with venetoclax+rituximab is based on an integrated assessment of safety, tolerability, PK, and preliminary evidence of clinical activity. The objective of the expansion portion is to gain additional safety, tolerability, PK, and preliminary efficacy data regarding the combination of APR-246 with venetoclax+rituximab therapy. Additional expansions may be added based on preliminary data obtained from the safety lead-in cohorts through a protocol amendment with appropriate justification.
7.5.2 Study Endpoints

Primary endpoints of the study are as follows: (1) Occurrence of dose limiting toxicity (DLT), classified and graded according to the National Cancer Institute's Common Terminology Criteria for Adverse Events (NCI-CTCAE, version 5.0); (2) Frequency of treatment-emergent adverse events (TEAEs), and serious adverse events (SAEs) related to APR-246 in combination with venetoclax+rituximab therapy; and (3) The highest dose of APR-246 with acceptable toxicity (the Recommended Phase II dose (RP2D) of APR-246) (the dose producing≤20% of DLT).

Secondary endpoints of the study include: (1) pharmacokinetic parameters: Cmax (maximum concentration), AUC (area under the curve), V_{d} and clearance (CL) of APR-246 and Cₘₐₓ (maximum concentration), Tₘₐₓ (time of maximum concentration), and AUC (area under the curve) of venetoclax+rituximab; (2) complete remission (CR) rate, defined as the proportion of subjects who achieve CR as per disease-specific response criteria; (3) overall response rate (ORR), defined as the proportion of subjects achieving a response, as per disease-specific response criteria; (4) Duration of response (DOR), defined as a time from documentation of tumor response to disease progression or death as a result of any cause; (5) progression-free survival (PFS), defined as the time from the first study dose date to the date of first documentation of confirmed disease progression or death (whichever occurs first).

The exploratory endpoints include: (1) Exploratory analyses of molecular markers for response predication and monitoring may include, but are not limited to: TP53 VAF by Next-generation sequencing (NGS), mutations in other genes by NGS, RNA expression; and (2) the exposure response relationship for safety and efficacy of APR-246 when combined with venetoclax+rituximab therapy.
7.5.3 Inclusion Criteria

Subjects must meet the inclusion criteria to be eligible to be enrolled, e.g., among other criteria, the subject must have at least one TP53 mutation that is not benign or likely benign based on local testing.

In the detailed inclusion criteria, the subject:
1. Is able to understand and is willing and able to comply with the study requirements and to provide written informed consent.
2. Has documented histologic diagnosis of R/R CLL or R/R MCL by WHO criteria with at least two prior systemic therapies. In R/R CLL, response and PD are assessed by iwCLL 2018 criteria; in R/R MCL, response and PD are assessed by Lugano criteria.
3. has most recent regimen that did not include Bcl-2 inhibitor therapy (e.g., venetoclax).
4. Has Prothrombin time (or international normalized ratio) and partial thromboplastin time not to exceed 1.2 times of the institution's normal range (patients with an elevated prothrombin time and known lupus anticoagulant may be eligible for participation after consulting the Medical Monitor).
5. Has adequate BM function independent of growth factor or transfusion support, per local laboratory reference range at screening as follows:
   a. platelet count≥75 000/mm³;
   b. ANC≥1000/mm³ unless cytopenia is clearly due to marrow involvement from CLL or MCL
   c. total hemoglobin≥9 g/dL (without transfusion support within 2 weeks of screening);
   d. If any of the above-mentioned cytopenias (a-c) are present due to significant BM involvement (requiring transfusion or G-CSF support) CLL/MCL patients may proceed with enrollment after discussion with the Medical Monitor. Cytopenias may not be due to evidence of MDS or hypoplastic BM.
6. Has adequate organ function as defined by the following laboratory values:
   a. Creatinine clearance≥30 mL/min (by Cockcroft-Gault method),
   b. Total serum bilirubin≤1.5×ULN unless due to Gilbert's syndrome, NHL organ involvement, controlled immune hemolysis or considered an effect of regular blood transfusions.
   c. ALT and AST≤2.5×ULN, unless due to NHL organ involvement.
7. Age >18 years at the time of signing the informed consent form.
8. At least one TP53 mutation which is not benign or likely benign as determined by the Medical Monitor based on local testing.
9. ECOG performance status of 0, 1 or 2.
10. Projected life expectancy of≥12 weeks.
11. Female patients must be surgically sterile, postmenopausal (for at least 1 year), or have negative results for a pregnancy test performed at screening, on a serum sample obtained within 7 days prior to initiation of study treatment.
12. Women of childbearing potential and men with female partners of childbearing potential must be willing to use an effective form of contraception.
   a. Patient who are enrolled should use an effective form of contraception for up to 30 days after the last dose of APR-246 in combination with venetoclax or up to 12 months after the last dose of rituximab, whichever time period is longer.
7.5.4 Treatment Administration

Study treatment is administered on an outpatient basis. No investigational or commercial agents or therapies other than those described herein is administered with the intent to treat the subject's disease.

Administration of APR-246

2-(Hydroxymethyl)-2-(methoxymethyl)quinuclidin-3-one (APR-246) is administered as a 6-hour IV infusion daily on days 1-4 of each 28-day cycle. The APR-246 starting dose is fixed at 4.5 g. APR-246 is administered in a 2-step infusion:
Step 1: Loading dose of 1.5 g for the first 45 minutes (±2 min); and
Step 2: Maintenance dose of 3 g over 5 hours 15 minutes (±30 min).

The dose of APR-246 may be reduced per the safety lead-in dose de-escalation procedure, or treatment interrupted if the subject develops AEs.

APR-246 vials are stored at 2-8° C. (35.6-46.4° F.). At the pharmacies and at the study centers, the prepared APR-246 study product (diluted in sodium chloride solution) are stored at not more than 25° C. The infusion is completed within 24 hours from the time of preparation.

Detailed instructions on vial concentration, preparation, and dispensing can be found in the Pharmacy Binder. The infusion timing, including start/stop times and the time of rate change are recorded.

Administration of Venetoclax

Prior to Cycle 1 for subjects enrolled in Safety Lead-In Cohort and Expansion cohort with venetoclax+rituximab in combination with APR-246, treatment with venetoclax start with a ramp-up phase as per the FDA label and be administered over a 5-week period (refer to Table 33 below). The 5-week ramp-up dosing schedule is designed to gradually reduce tumor burden (debulk) and decrease the risk of TLS. After the ramp-up phase is completed, venetoclax tablets at a dose of 400 mg are taken PO once daily with a meal and water, at approximately the same time every day. Note that On Day 1 of Cycle 1, venetoclax is taken 1 hour prior to administration of the APR-246 infusion.

TABLE 33
Dosing Schedule for Ramp-Up Phase in Patients with CLL

| Week Number | Venetoclax Daily Dose |
|---|---|
| Week 1 | 20 mg |
| Week 2 | 50 mg |
| Week 3 | 100 mg |
| Week 4 | 200 mg |
| Week 5 and beyond | 400 mg |

If required, the dosage of venetoclax is modified. Detailed instructions on administration of venetoclax can be found in the Pharmacy Binder and current USPI.

Administration of Rituximab

Rituximab is given on Day 5 of each cycle for a maximum of 6 cycles. Rituximab is given at a dose of 375 mg/m² in Cycle 1 and 500 mg/m² for Cycles 2 through 6.

Subjects may remain on study treatment to the end of the trial while deriving clinical benefit, unless unacceptable toxicity, progression, death or subject withdrawal. Subjects may remain on study treatment after disease progression if they are continuing to derive clinical benefit in the opinion of the investigator.

Dose Limiting Toxicity

A DLT is defined as any of the TEAEs defined by the National Cancer Institute's Common Terminology Criteria for Adverse Events, version 5.0 (NCI-CTCAE v5.0), and for hematologic AEs in R/R CLL/MCL patients, iwCLL criteria for CLL for hematologic toxicity, occurring during the first 28 days of study drug, unless the AE is clearly unrelated to study drug (related to the patient's disease under study, their medical condition or concomitant medications, or clearly attributable to venetoclax or rituximab).

DLT definitions are:
Any≥Grade 3 non-hematologic toxicity except for:
   First occurrence of Grade 3 electrolyte abnormalities and/or creatinine clearance decrease resolving to Grade 2 (or baseline if baseline is≥Grade 2) within 48 hours with supportive treatment.
   Grade 3 fatigue, nausea, vomiting, diarrhea or other manageable constitutional symptom that is responsive to supportive therapy.
   Grade 3 infection responding to appropriate antimicrobial therapy.
   Any neurologic toxicity of grade 4, or grade 3 that does not return to grade 1 or baseline within 7 days.
Any≥Grade 3 hematologic toxicity are considered a DLT except for:
   Grade 3 neutropenia without fever
   Grade 4 neutropenia without fever lasting 8 days or less
   Grade 3 thrombocytopenia that does not result in bleeding or transfusion
   Grade 3/4 lymphopenia/lymphocytosis
   Grade 3/4 WBC decreased
   Grade 3/4 WBC increased

Any toxicity, regardless of the NCI-CTCAE v5.0 grade, resulting in discontinuation, dose reduction or treatment with less than 75% of planned doses of APR-246 study drug, are reviewed by the DRT, and a considered a DLT if determined that the toxicity is clearly related to study drug, unless reversible CNS-related effects previously described for APR-246 or related to the patient's underlying disease, other medical condition or concomitant medications, or clearly attributable to venetoclax or rituximab.

G-CSF support for the management of neutropenia is allowed including during the DLT period.

Recommended Phase II Dose

The Recommended Phase II Dose (RP2D) of APR-246 is defined as the dose at which less than 2 out of 6 subjects experience DLT during the 4-week safety assessment period after administration of APR-246 in combination with venetoclax. Up to 10 additional patients may be enrolled at the RP2D to confirm the confidence at that dose level.

Data Review Team (DRT) consisting of the Medical Monitor, Site Principal Investigators, and other clinical research personnel that the Sponsor may deem appropriate, hold Data Review Meetings (DRMs) on an interim basis at a frequency dependent on study accrual. At these meetings, the DRT review AEs and DLTs and make recommendations regarding the RP2D. In the expansion portion of the study, the DRT evaluate safety and tolerability after 5 subjects have completed 1 cycle of treatment in each cohort. All accumulated safety data are discussed during DRMs.

7.5.5 Statistics

Demographic data and disease-related characteristics are summarized using descriptive statistics (count and percent, mean, median, standard deviation, minimum, maximum). Continuous variables are presented by n, mean, median, standard deviation and range (minimum and maximum), and categorical variables are presented by count and percentage of subjects as appropriate. Data are presented by each dose cohort in safety lead-in dose-finding portion and by each treatment arm and dose cohort in the expansion portion. All subject data, efficacy and safety data are summarized.

Sample Size

A total of approximately 120 evaluable patients are included in the study. In the Safety Lead-In portion of the study, two cohorts enroll subjects with TP53-mutant R/R CLL in a 3+3 dose de-escalation design. A maximal of 6×6×6 (18) DLT evaluable patients are included in this portion of the study by allowing 2 APR-246 dose reductions for the 2 cohorts. An additional up to 10 patients are enrolled at the RP2D to confirm the safety at that dose level.

In the Expansion cohort, 20 patients with R/R CLL and 40 patients with R/R MCL are included to further investigate the safety profile at RP2D and efficacy effects.

Analysis Populations

Safety population: Subjects are evaluable for safety if they receive at least one dose of APR-246 with venetoclax+rituximab. The safety population is the primary analysis population used for all analyses such as patient disposition, patient demographics, exposure, safety parameters, and efficacy parameters. The safety population is the primary analysis population for efficacy.

Efficacy evaluable (EE) population: All subjects who complete at least one treatment cycle of APR-246 and venetoclax+rituximab and who have at least one post treatment clinical response assessment. Subjects who fail to complete one treatment cycle is also considered EE if they show clear evidence of clinically significant disease progression. The EE population is the secondary analysis population for efficacy.

Pharmacokinetic (PK) population: Subjects is evaluable for PK if at least one sample for PK evaluation has been obtained

Safety Analysis

Safety data are summarized for the safety population. These data include adverse events and laboratory parameters. AE terms are coded using the Medical Dictionary for Drug Regulatory Activities (MedDRA)®, version 22.0 or higher. AEs are summarized by System Organ Class (SOC), preferred term, severity, and relationship to treatment. Serious adverse events (SAEs), deaths, and adverse events (AEs) leading to early discontinuation of study treatment are summarized. Laboratory parameters are summarized by maximum NCI-CTCAE version 5.0 severity grade and also by change from pre-treatment to scheduled time points using descriptive statistics. Laboratory parameter listings include the normal ranges for each parameter. Each value is classified as falling above, below, or within the normal range.

Only AEs related to study screening procedures are collected from the time of signing informed consent, throughout study enrollment, and up to 30 days after last dose. Data summaries include only treatment-emergent adverse events (TEAEs), defined as events occurring at the start of APR-246 infusion on Day 1, Cycle 1 up to and including 30 days after last dose of study treatment.

Efficacy

Overall response rate (ORR), defined as the proportion of subjects achieving complete remission (CR) or partial remission (PR) measure per Lugano criteria (see, e.g., van Heertum, R. L., et al, Drug. Des. Devel. Ther. 11, 1719-1728 (2017)) for NHL (MCL), or iwCLL 2018 criteria for patients with CLL (see, e.g., Hallek, M., et al., Blood 131(25), 2745-2760 (2018)). Both Overall response rate and complete remission with exact 95% CI are summarized by cohort.

Duration of response (DoR) is defined as the time from the date when criteria for response are met to the date of progressive disease (PD) or death due to any cause, whichever occurs first. Subjects alive with no progressive disease (PD) has their DOR censored at the date of the last clinical assessment. The duration of complete remission (CR) are summarized in each treatment arm by providing the median DOR together with associated 95% CI, using Kaplan-Meier methodology. DOR endpoints are not formally compared between treatment arms.

Overall response is summarized in number (%) of subjects in each category of responses and ORR is analyzed by using the similar method as CR rate.

Survival data are collected at treatment and follow-up periods. Subjects are followed until death. Overall survival (OS) is defined as the number of days from the first day of treatment to the date of death. Kaplan-Meier methodology is utilized.

Progression-free survival (PFS) is defined as the time from the first day of treatment to disease progression or death from MDS, whichever occurs first. If neither event occurs, PFS is censored at the date of the last clinical assessment. Kaplan-Meier methodology is utilized.

Pharmacokinetic Analysis

The pharmacokinetics of APR-246 and venetoclax are summarized using descriptive statistics (mean, standard deviation, CV % mean, geometric mean, CV % geometric mean) and compared with historical control data.

Concentrations of APR-246 and venetoclax are determined, and pharmacokinetic parameters (e.g., Cₘₐₓ, Tₘₐₓ, AUC, V_{d} and CL) are derived using popPK or non-compartmental methods.

APR-246 AUC and Cmax are then be tested for association with signs of efficacy and safety. If an observable trend exists, a PK/PD model is developed to evaluate the exposure-response relationship between APR-246 plasma exposure and outcome measures. Demographic and clinical data (ethnicity, current age, body weight, sex, disease status, etc.) are utilized to assess intersubject variability in the PK and PK/PD relationships.

Exploratory Analyses

Descriptive statistics/results from exploratory analyses of molecular markers for response predication and monitoring are prepared and may include but are not limited to: TP53 VAF by Next-generation sequencing (NGS), mutations in other genes by NGS, RNA expression.
7.5.6 TP53 Sequencing—Variant Interpretation Algorithm

Inclusion of patients in the study is based on TP53 sequencing performed in a laboratory at each participating site according to established local routines. A study-specific variant interpretation algorithm is used to discriminate between eligible and non-eligible TP53 sequence variants.

In order to select patients with high unmet medical need due to poor prognosis, the study will enroll patients that have any TP53 mutation which is not pre-defined as “benign” or “likely benign.” Eligible TP53 mutations herein includes, for example, variants classified as pathogenic, likely pathogenic and variant of uncertain significance (VUS) in a specified database (UMD-TP53). Patients harboring at least one such TP53 sequence variant are eligible for inclusion, while patients who only have variant(s) classified as benign or likely benign are not eligible. Thus, also TP53 VUS are eligible, avoiding exclusion of patients with possible pathogenic TP53 variants presently classified as VUS.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed:

1. A method of treating a hyperproliferative malignancy in a subject, comprising administering to the subject a therapeutically effective amount of a compound that can give reactivation of a mutant p53 and an inhibitor of an antiapoptotic Bcl-2 family protein.

2. The method of claim 1, wherein the compound that can give reactivation of the mutant p53 promotes proper folding of the mutant p53 and restores at least part of a normal p53 function.

3. The method of claim 1, wherein the compound is resting in a shift of the equilibrium from unfolded towards a wild-type like p53 conformation.

4. The method of claim 1, wherein the compound that can give reactivation of the mutant p53 interfers with aggregation of misfolded mutant p53 or reduce aggregation of the mutant p53.

5. The method of claim 1, wherein the compound or its metabolite or degradation product thereof restores a p53 wild type function by covalent binding to the mutant p53.

6. The method of claim 5, wherein the compound binds to thiol groups in the core domain of the mutant p53 and restore wild-type conformation.

7. The method of any one of claims 1-6, wherein the mutant p53 comprises at least one of the replacements R175H or R273H.

8. The method of claim 1, wherein the compound that reactivates the mutant p53 is selected from the group consisting of:
2-(hydroxymethyl)-2-(methoxymethyl)quinuclidin-3-one;
2,2-bis(hydroxymethyl)quinuclidin-3-one;
2,2,2-trichloro-N-ethyl-N-((3-oxoquinuclidin-2-yl)methyl)acetamide;
2,2,2-trichloro-N-((3-oxoquinuclidin-2-yl)methyl)acetamide;
N-ethyl-2,2,2-trifluoro-N-((3-oxoquinuclidin-2-yl)methyl)acetamide;
2,2,2-trifluoro-N-((3-oxoquinuclidin-2-yl)methyl)acetamide;
2,2-difluoro-N-((3-oxoquinuclidin-2-yl)methyl)acetamide,
N-((3-oxoquinuclidin-2-yl)methyl)pyridine-3-sulfonamide;
4-fluoro-N-((3-oxoquinuclidin-2-yl)methyl)benzenesulfonamide;
N-ethyl-N-((3-oxoquinuclidin-2-yl)methyl)methanesulfonamide;
N-((3-oxoquinuclidin-2-yl)methyl)methanesulfonamide;
N-((3-oxoquinuclidin-2-yl)methyl)benzenesulfonamide;
2-(N-((3-oxoquinuclidin-2-yl)methyl)methylsulfonamido)acetamide;
N-(methylsulfonyl)-N-((3-oxoquinuclidin-2-yl)methyl)glycine;
N-((3-oxoquinuclidin-2-yl)methyl)pyridine-4-sulfonamide;
N-((3-oxoquinuclidin-2-yl)methyl)pyridine-2-sulfonamide;
N-ethyl-1,1,1-trifluoro-N-((3-oxoquinuclidin-2-yl)-methyl)methanesulfonamide;
1,1,1-trifluoro-N-((3-oxoquinuclidin-2-yl)methyl)methanesulfonamide;
N,N-bis((3-oxoquinuclidin-2-yl)methyl)methanesulfonamide;
N-((3-oxoquinuclidin-2-yl)methyl)propane-2-sulfonamide;
N-((3-oxoquinuclidin-2-yl)methyl)cyclopropanesulfonamide;
1-methyl-N-((3-oxoquinuclidin-2-yl)methyl)cyclopropane-1-sulfonamide;
N-cyclopropyl-N-((3-oxoquinuclidin-2-yl)methyl)methanesulfonamide;
N-((3-oxoquinuclidin-2-yl)methyl)-N-phenylmethanesulfonamide;
1-((3-oxoquinuclidin-2-yl)methyl)pyrimidine-2,4(1H,3H)-dione;
5-methyl-1-((3-oxoquinuclidin-2-yl)methyl)pyrimidine-2,4(1H,3H)-dione;
tert-butyl 5-methyl-2,6-dioxo-3-((3-oxoquinuclidin-2-yl)methyl)-3,6-dihydropyrimidine-1(2H)-carboxylate;
5-methyl-1,3-bis((3-oxoquinuclidin-2-yl)methyl)pyrimidine-2,4(1H,3H)-dione;
N-methyl-1-((3-oxoquinuclidin-2-yl)methyl)-1H-1,2,4-triazole-3-carboxamide;
2-((3-chloro-1H-1,2,4-triazol-1-yl)methyl)quinuclidin-3-one;
N,N-dimethyl-1-((3-oxoquinuclidin-2-yl)methyl)-1H-1,2,4-triazole-3-carboxamide;
2-((1H-1,2,4-triazol-1-yl)methyl)quinuclidin-3-one;
1-((3-oxoquinuclidin-2-yl)methyl)-1H-1,2,4-triazole-3-carbonitrile; and
1-((3-oxoquinuclidin-2-yl)methyl)-1H-1,2,4-triazole-3-carboxamide,
or a pharmaceutically acceptable salt thereof.

9. The method of claim 8, wherein the compound is 2-(hydroxymethyl)-2-(methoxymethyl) quinuclidin-3-one (APR-246) having the following formula: 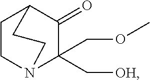
or a pharmaceutically acceptable salt thereof.

10. The method of claim 8, wherein the compound is 2,2,2-trifluoro-N-((3-oxoquinuclidin-2-yl)methyl)acetamide (Compound A) having the following formula: 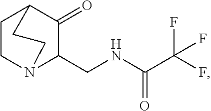 [SMILES: O=C1C2CCN(CC2)C1CNC(=O)C(F)(F)F]
or a pharmaceutically acceptable salt thereof.

11. The method of claim 8, wherein the compound is 2,2,2-trichloro-N-ethyl-N-((3-oxoquinuclidin-2-yl)methyl)acetamide (Compound B) having the following formula: 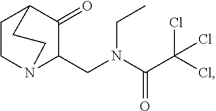 [SMILES: CCN(CC1C(=O)C2CCN1CC2)C(=O)C(Cl)(Cl)Cl]
or a pharmaceutically acceptable salt thereof.

12. The method of claim 8, wherein the compound is 2,2,2-trichloro-N-((3-oxoquinuclidin-2-yl)methyl)acetamide (Compound C) having the following formula: 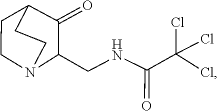 [SMILES: O=C1C2CCN(CC2)C1CNC(=O)C(Cl)(Cl)Cl]
or a pharmaceutically acceptable salt thereof.

13. The method of claim 8, wherein the compound is N-ethyl-2,2,2-trifluoro-N-((3-oxoquinuclidin-2-yl)methyl)acetamide (Compound D) having the following formula: 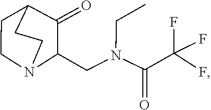 [SMILES: CCN(CC1C(=O)C2CCN1CC2)C(=O)C(F)(F)F]
or a pharmaceutically acceptable salt thereof.

14. The method of claim 8, wherein the compound is 2,2-difluoro-N-((3-oxoquinuclidin-2-yl)methyl)acetamide (Compound E) having the following formula: 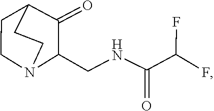 [SMILES: O=C(NCC1C(=O)C2CCN1CC2)C(F)F]
or a pharmaceutically acceptable salt thereof.

15. The method of any one of claims 1-14, wherein the inhibitor of an antiapoptotic Bcl-2 family protein is a Bcl-2 inhibitor.

16. The method of claim 15, wherein the Bcl-2 inhibitor is selected from the group consisting of venetoclax (ABT-199), navitoclax, oblimersen, PNT2258, and SPC2996.

17. The method of claim 16, wherein the Bcl-2 inhibitor is venetoclax (ABT-199).

18. The method of claim 1, wherein the method comprises administering to the subject a therapeutically effectively amount of APR-246 and venetoclax (ABT-199).

19. The method of claim 1, wherein the method comprises administering to the subject a therapeutically effectively amount of Compound A and venetoclax (ABT-199).

20. The method of claim 1, wherein the method comprises administering to the subject a therapeutically effectively amount of Compound B and venetoclax (ABT-199).

21. The method of claim 1, wherein the method comprises administering to the subject a therapeutically effectively amount of Compound C and venetoclax (ABT-199).

22. The method of claim 1, wherein the method comprises administering to the subject a therapeutically effectively amount of Compound D and venetoclax (ABT-199).

23. The method of claim 1, wherein the method comprises administering to the subject a therapeutically effectively amount of Compound E and venetoclax (ABT-199).

24. The method of any one of claims 1-18, wherein the inhibitor of an antiapoptotic Bcl-2 family protein is a Mcl-1 inhibitor.

25. The method of claim 24, wherein the Mcl-1 inhibitor is selected from the group consisting of AT101, TW-37, Gambogic acid, Sabutoclax (BI-97C1), Marinopyrrole A (maritoclax), UMI-77, A-1210477, MIK665, AMG-176, AZD5991, Flavopiridol, Roscovitine, CR8, Voruciclib (P1446A-05), Cardiac glycosides UNBS1450, Benzyl isothiocyanate, BAY43-9006, BEZ235 AZD8055, and Arsenic trioxide Bufalin.

26. The method of claim 25, wherein the Mcl-1 inhibitor is AMG-176.

27. The method of claim 25, wherein the Mcl-1 inhibitor is MIK665.

28. The method of claim 1, wherein the method comprises administering to the subject a therapeutically effectively amount of APR-246 and AMG-176.

29. The method of claim 1, wherein the method comprises administering to the subject a therapeutically effectively amount of APR-246 and MIK665.

30. The method of any one of claims 129, wherein the p53 reactivator is formulated in a first pharmaceutical composition and the inhibitor of an antiapoptotic Bcl-2 family protein is formulated in a second pharmaceutical composition.

31. The method of any one of claims 1-30, further comprising administering to the subject an additional agent.

32. The method of claim 31, wherein the additional agent is a hypomethylating agent.

33. The method of claim 32, wherein the additional agent is Azacitidine.

34. The method of claim 31, wherein the additional agent is an anti-CD20 antibody.

35. The method of claim 34, wherein the additional agent is rituximab.

36. The method of any one of claims 1-35, wherein the hyperproliferative malignancy is a hematological malignancy.

37. The method of claim 36, wherein the hematological malignancy is leukemia, lymphoma, or myeloma.

38. The method of any one of claims 1-35, wherein the hematological malignancy is selected from the group consisting of: Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia.

39. The method of claim 38, wherein the hematologic malignancy is myelodysplastic syndromes (MDS).

40. The method of claim 38, wherein the hematologic malignancy is acute myeloid leukemia (AML).

41. The method of claim 38, wherein the hematologic malignancy is chronic lymphocytic leukemia (CLL).

42. The method of claim 38, wherein the hematologic malignancy is multiple myeloma (MM).

43. The method of any one of claims 1-35, wherein the hyperproliferative malignancy is a solid tumor cancer.

44. The method of claim 43, wherein the solid tumor cancer is selected from the group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

45. The method of any one of claims 1-44, wherein the hyperproliferative malignancy comprises a cancer cell having mutant p53.

46. The method of any one of claims 1-44, wherein the hyperproliferative malignancy does not comprise a cancer cell having mutant p53.

47. The method of any one of claims 1-44, wherein the hyperproliferative malignancy comprises a cancer cell having wild type p53.

48. A method of treating hyperproliferative malignancy in a subject, comprising administering to the subject a therapeutically effective amount of a compound that can give reactivation of a mutant p53, wherein the hyperproliferative malignancy does not comprise a cancer cell having mutant p53 or the hyperproliferative malignancy comprises a cancer cell having wild type p53.

49. The method of claim 48, wherein the compound is APR-246.

50. The method of claim 48, wherein the compound is Compound A.

51. The method of claim 48, wherein the compound is Compound B.

52. The method of claim 48, wherein the compound is Compound C.

53. The method of claim 48, wherein the compound is Compound D.

54. The method of claim 48, wherein the compound is Compound E.


SEQUENCE LISTING (4 sequences)

SEQ ID NO: 1 - protein, 9 aa, Artificial Sequence
  Engineered p53 activator peptide
  LIPID (1)..(1): MYRISTATE
RRHSTPHPD

SEQ ID NO: 2 - protein, 9 aa, Artificial Sequence
  Engineered p53 activator peptide
REDEDEIEW

SEQ ID NO: 3 - DNA, 25 bp, Artificial Sequence
  Engineered p53 binding aptamer
attagcgcattttaacatagggtgc

SEQ ID NO: 4 - protein, 16 aa, Artificial Sequence
  Engineered p53 activator peptide
RRRRRRRRRLTRITLE